SEQUENCE LISTING (32 sequences)

SEQ ID NO: 1 - protein, 306 aa, unidentified
  source 1..306: note = Bacillus cytotoxicus
MQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGSIKASVTPSGPSGESGATGQVTWSDSVSYKQTSYKTNLIDQTNKHVKWNVFFNGYNNQNWGIYTRDSYHALYGNQLFMYSRTYPHETDARGNLVPMNDLPALTNSGFSPGMIAVVISEKDTEQSSIQVAYTKHADDYTLRPGFTFGTGNWVGNNIKDVDQKTFNKSFVLDWKNKKLVEKK

SEQ ID NO: 2 - DNA, 924 bp, unidentified
  source 1..924: mol_type = genomic DNA; note = Bacillus cytotoxicus
atgcaaaccacctcccaagtcgtcacggacatcggtcagaacgctaaaacccataccagctacaataccttcaataacgaacaagcagataacatgaccatgagcctgaaagtcacgtttattgatgacccgtctgcagataagcagattgctgttatcaacaccacgggctcattcatgaaagcaaatccgacgctgtcggatgctccggtggacggttatccgattccgggtgctagtgttaccctgcgttatccgtcccagtacgatatcgcgatgaacctgcaagacaataccagtcgctttttccatgtggcgccgacgaatgccgttgaagaaaccacggtcaccagctctgtgagctatcagctgggcggtagcatcaaagcctctgtgaccccgtctggtccgagtggtgaatccggtgcaaccggtcaagtcacgtggtcagatagcgtgagctataaacagaccagctacaagacgaacctgattgaccaaaccaataaacacgttaagtggaacgtctttttcaatggctataacaatcagaactggggtatctacacccgtgatagttatcatgccctgtacggcaatcaactgtttatgtattcccgtacctacccgcacgaaacggatgcgcgcggtaacctggtgccgatgaatgacctgccggccctgaccaactcaggcttctcgccgggtatgattgcagtggttatctctgaaaaagataccgaacagagttccattcaagttgcgtataccaagcatgccgatgactacacgctgcgtccgggttttaccttcggtacgggtaattgggttggtaacaatatcaaagatgtcgaccagaaaaccttcaataaatcgttcgtgctggactggaaaaataagaaactggtggaaaagaaataatga

SEQ ID NO: 3 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 4 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 5 - protein, 425 aa, Thermus thermophilus
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 6 - protein, 226 aa, unidentified
  source 1..226: note = bacteriophage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 7 - protein, 608 aa, unidentified
  source 1..608: note = Bacillus subtilis bacteriophage phi29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 8 - protein, 760 aa, Methanococcoides burtonii
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 9 - protein, 707 aa, unidentified
  source 1..707: note = Cenarchaeum symbiosum
MRISELDIPRPAIEFLEGEGYKKLYPPQAAAAKAGLTDGKSVLVSAPTASGKTLIAAIAMISHLSRNRGKAVYLSPLRALAAEKFAEFGKIGGIPLGRPVRVGVSTGDFEKAGRSLGNNDILVLTNERMDSLIRRRPDWMDEVGLVIADEIHLIGDRSRGPTLEMVLTKLRGLRSSPQVVALSATISNADEIAGWLDCTLVHSTWRPVPLSEGVYQDGEVAMGDGSRHEVAATGGGPAVDLAAESVAEGGQSLIFADTRARSASLAAKASAVIPEAKGADAAKLAAAAKKIISSGGETKLAKTLAELVEKGAAFHHAGLNQDCRSVVEEEFRSGRIRLLASTPTLAAGVNLPARRVVISSVMRYNSSSGMSEPISILEYKQLCGRAGRPQYDKSGEAIVVGGVNADEIFDRYIGGEPEPIRSAMVDDRALRIHVLSLVTTSPGIKEDDVTEFFLGTLGGQQSGESTVKFSVAVALRFLQEEGMLGRRGGRLAATKMGRLVSRLYMDPMTAVTLRDAVGEASPGRMHTLGFLHLVSECSEFMPRFALRQKDHEVAEMMLEAGRGELLRPVYSYECGRGLLALHRWIGESPEAKLAEDLKFESGDVHRMVESSGWLLRCIWEISKHQERPDLLGELDVLRSRVAYGIKAELVPLVSIKGIGRVRSRRLFRGGIKGPGDLAAVPVERLSRVEGIGATLANNIKSQLRKGG

SEQ ID NO: 10 - protein, 720 aa, unidentified
  source 1..720: note = Thermococcus gammatolerans EJ3
MKVDELPVDERLKAVLKERGIEELYPPQAEALKSGALEGRNLVLAIPTASGKTLVSEIVMVNKLIQEGGKAVYLVPLKALAEEKYREFKEWEKLGLKVAATTGDYDSTDDWLGRYDIIVATAEKFDSLLRHGARWINDVKLVVADEVHLIGSYDRGATLEMILTHMLGRAQILALSATVGNAEELAEWLDASLVVSDWRPVQLRRGVFHLGTLIWEDGKVESYPENWYSLVVDAVKRGKGALVFVNTRRSAEKEALALSKLVSSHLTKPEKRALESLASQLEDNPTSEKLKRALRGGVAFHHAGLSRVERTLIEDAFREGLIKVITATPTLSAGVNLPSFRVIIRDTKRYAGFGWTDIPVLEIQQMMGRAGRPRYDKYGEAIIVARTDEPGKLMERYIRGKPEKLFSMLANEQAFRSQVLALITNFGIRSFPELVRFLERTFYAHQRKDLSSLEYKAKEVVYFLIENEFIDLDLEDRFIPLPFGKRTSQLYIDPLTAKKFKDAFPAIERNPNPFGIFQLIASTPDMATLTARRREMEDYLDLAYELEDKLYASIPYYEDSRFQGFLGQVKTAKVLLDWINEVPEARIYETYSIDPGDLYRLLELADWLMYSLIELYKLFEPKEEILNYLRDLHLRLRHGVREELLELVRLPNIGRKRARALYNAGFRSVEAIANAKPAELLAVEGIGAKILDGIYRHLGIEKRVTEEKPKRKGTLEDFLR

SEQ ID NO: 11 - protein, 799 aa, unidentified
  source 1..799: note = Methanospirillum hungatei JF-1
MEIASLPLPDSFIRACHAKGIRSLYPPQAECIEKGLLEGKNLLISIPTASGKTLLAEMAMWSRIAAGGKCLYIVPLRALASEKYDEFSKKGVIRVGIATGDLDRTDAYLGENDIIVATSEKTDSLLRNRTPWLSQITCIVLDEVHLIGSENRGATLEMVITKLRYTNPVMQIIGLSATIGNPAQLAEWLDATLITSTWRPVDLRQGVYYNGKIRFSDSERPIQGKTKHDDLNLCLDTIEEGGQCLVFVSSRRNAEGFAKKAAGALKAGSPDSKALAQELRRLRDRDEGNVLADCVERGAAFHHAGLIRQERTIIEEGFRNGYIEVIAATPTLAAGLNLPARRVIIRDYNRFASGLGMVPIPVGEYHQMAGRAGRPHLDPYGEAVLLAKDAPSVERLFETFIDAEAERVDSQCVDDASLCAHILSLIATGFAHDQEALSSFMERTFYFFQHPKTRSLPRLVADAIRFLTTAGMVEERENTLSATRLGSLVSRLYLNPCTARLILDSLKSCKTPTLIGLLHVICVSPDMQRLYLKAADTQLLRTFLFKHKDDLILPLPFEQEEEELWLSGLKTALVLTDWADEFSEGMIEERYGIGAGDLYNIVDSGKWLLHGTERLVSVEMPEMSQVVKTLSVRVHHGVKSELLPLVALRNIGRVRARTLYNAGYPDPEAVARAGLSTIARIIGEGIARQVIDEITGVKRSGIHSSDDDYQQKTPELLTDIPGIGKKMAEKLQNAGIITVSDLLTADEVLLSDVLGAARARKVLAFLSNSEKENSSSDKTEEIPDTQKIRGQSSWEDFGC

SEQ ID NO: 12 - protein, 1756 aa, Escherichia coli
MMSIAQVRSAGSAGNYYTDKDNYYVLGSMGERWAGKGAEQLGLQGSVDKDVFTRLLEGRLPDGADLSRMQDGSNKHRPGYDLTFSAPKSVSMMAMLGGDKRLIDAHNQAVDFAVRQVEALASTRVMTDGQSETVLTGNLVMALFNHDTSRDQEPQLHTHAVVANVTQHNGEWKTLSSDKVGKTGFIENVYANQIAFGRLYREKLKEQVEALGYETEVVGKHGMWEMPGVPVEAFSGRSQAIREAVGEDASLKSRDVAALDTRKSKQHVDPEIRMAEWMQTLKETGFDIRAYRDAADQRTEIRTQAPGPASQDGPDVQQAVTQAIAGLSERKVQFTYTDVLARTVGILPPENGVIERARAGIDEAISREQLIPLDREKGLFTSGIHVLDELSVRALSRDIMKQNRVTVHPEKSVPRTAGYSDAVSVLAQDRPSLAIVSGQGGAAGQRERVAELVMMAREQGREVQIIAADRRSQMNLKQDERLSGELITGRRQLLEGMAFTPGSTVIVDQGEKLSLKETLTLLDGAARHNVQVLITDSGQRTGTGSALMAMKDAGVNTYRWQGGEQRPATIISEPDRNVRYARLAGDFAASVKAGEESVAQVSGVREQAILTQAIRSELKTQGVLGHPEVTMTALSPVWLDSRSRYLRDMYRPGMVMEQWNPETRSHDRYVIDRVTAQSHSLTLRDAQGETQVVRISSLDSSWSLFRPEKMPVADGERLRVTGKIPGLRVSGGDRLQVASVSEDAMTVVVPGRAEPASLPVSDSPFTALKLENGWVETPGHSVSDSATVFASVTQMAMDNATLNGLARSGRDVRLYSSLDETRTAEKLARHPSFTVVSEQIKARAGETLLETAISLQKAGLHTPAQQAIHLALPVLESKNLAFSMVDLLTEAKSFAAEGTGFTELGGEINAQIKRGDLLYVDVAKGYGTGLLVSRASYEAEKSILRHILEGKEAVTPLMERVPGELMETLTSGQRAATRMILETSDRFTVVQGYAGVGKTTQFRAVMSAVNMLPASERPRVVGLGPTHRAVGEMRSAGVDAQTLASFLHDTQLQQRSGETPDFSNTLFLLDESSMVGNTEMARAYALIAAGGGRAVASGDTDQLQAIAPGQSFRLQQTRSAADVVIMKEIVRQTPELREAVYSLINRDVERALSGLESVKPSQVPRLEGAWAPEHSVTEFSHSQEAKLAEAQQKAMLKGEAFPDIPMTLYEAIVRDYTGRTPEAREQTLIVTHLNEDRRVLNSMIHDAREKAGELGKEQVMVPVLNTANIRDGELRRLSTWEKNPDALALVDNVYHRIAGISKDDGLITLQDAEGNTRLISPREAVAEGVTLYTPDKIRVGTGDRMRFTKSDRERGYVANSVWTVTAVSGDSVTLSDGQQTRVIRPGQERAEQHIDLAYAITAHGAQGASETFAIALEGTEGNRKLMAGFESAYVALSRMKQHVQVYTDNRQGWTDAINNAVQKGTAHDVLEPKPDREVMNAQRLFSTARELRDVAAGRAVLRQAGLAGGDSPARFIAPGRKYPQPYVALPAFDRNGKSAGIWLNPLTTDDGNGLRGFSGEGRVKGSGDAQFVALQGSRNGESLLADNMQDGVRIARDNPDSGVVVRIAGEGRPWNPGAITGGRVWGDIPDNSVQPGAGNGEPVTAEVLAQRQAEEAIRRETERRADEIVRKMAENKPDLPDGKTELAVRDIAGQERDRSAISERETALPESVLRESQREREAVREVARENLLQERLQQMERDMVRDLQKEKTLGGD

SEQ ID NO: 13 - protein, 726 aa, Methanococcoides burtonii
MSDKPAFMKYFTQSSCYPNQQEAMDRIHSALMQQQLVLFEGACGTGKTLSALVPALHVGKMLGKTVIIATNVHQQMVQFINEARDIKKVQDVKVAVIKGKTAMCPQEADYEECSVKRENTFELMETEREIYLKRQELNSARDSYKKSHDPAFVTLRDELSKEIDAVEEKARGLRDRACNDLYEVLRSDSEKFREWLYKEVRSPEEINDHAIKDGMCGYELVKRELKHADLLICNYHHVLNPDIFSTVLGWIEKEPQETIVIFDEAHNLESAARSHSSLSLTEHSIEKAITELEANLDLLADDNIHNLFNIFLEVISDTYNSRFKFGERERVRKNWYDIRISDPYERNDIVRGKFLRQAKGDFGEKDDIQILLSEASELGAKLDETYRDQYKKGLSSVMKRSHIRYVADFMSAYIELSHNLNYYPILNVRRDMNDEIYGRVELFTCIPKNVTEPLFNSLFSVILMSATLHPFEMVKKTLGITRDTCEMSYGTSFPEEKRLSIAVSIPPLFAKNRDDRHVTELLEQVLLDSIENSKGNVILFFQSAFEAKRYYSKIEPLVNVPVFLDEVGISSQDVREEFFSIGEENGKAVLLSYLWGTLSEGIDYRDGRGRTVIIIGVGYPALNDRMNAVESAYDHVFGYGAGWEFAIQVPTIRKIRQAMGRVVRSPTDYGARILLDGRFLTDSKKRFGKFSVFEVFPPAERSEFVDVDPEKVKYSLMNFFMDNDEQ

SEQ ID NO: 14 - protein, 439 aa, unidentified
  source 1..439: note = Enterobacteria phage T4
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 15 - protein, 970 aa, unidentified
  source 1..970: note = Citromicrobium bathyomarinum JL354
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 16 - protein, 1830 aa, unidentified
  source 1..1830: note = Bacillus subtilis bacteriophage phi29
ATGAAACACATGCCGCGTAAAATGTATAGCTGCGCGTTTGAAACCACGACCAAAGTGGAAGATTGTCGCGTTTGGGCCTATGGCTACATGAACATCGAAGATCATTCTGAATACAAAATCGGTAACAGTCTGGATGAATTTATGGCATGGGTGCTGAAAGTTCAGGCGGATCTGTACTTCCACAACCTGAAATTTGATGGCGCATTCATTATCAACTGGCTGGAACGTAATGGCTTTAAATGGAGCGCGGATGGTCTGCCGAACACGTATAATACCATTATCTCTCGTATGGGCCAGTGGTATATGATTGATATCTGCCTGGGCTACAAAGGTAAACGCAAAATTCATACCGTGATCTATGATAGCCTGAAAAAACTGCCGTTTCCGGTGAAGAAAATTGCGAAAGATTTCAAACTGACGGTTCTGAAAGGCGATATTGATTATCACAAAGAACGTCCGGTTGGTTACAAAATCACCCCGGAAGAATACGCATACATCAAAAACGATATCCAGATCATCGCAGAAGCGCTGCTGATTCAGTTTAAACAGGGCCTGGATCGCATGACCGCGGGCAGTGATAGCCTGAAAGGTTTCAAAGATATCATCACGACCAAAAAATTCAAAAAAGTGTTCCCGACGCTGAGCCTGGGTCTGGATAAAGAAGTTCGTTATGCCTACCGCGGCGGTTTTACCTGGCTGAACGATCGTTTCAAAGAAAAAGAAATTGGCGAGGGTATGGTGTTTGATGTTAATAGTCTGTATCCGGCACAGATGTACAGCCGCCTGCTGCCGTATGGCGAACCGATCGTGTTCGAGGGTAAATATGTTTGGGATGAAGATTACCCGCTGCATATTCAGCACATCCGTTGTGAATTTGAACTGAAAGAAGGCTATATTCCGACCATTCAGATCAAACGTAGTCGCTTCTATAAGGGTAACGAATACCTGAAAAGCTCTGGCGGTGAAATCGCGGATCTGTGGCTGAGTAACGTGGATCTGGAACTGATGAAAGAACACTACGATCTGTACAACGTTGAATACATCAGCGGCCTGAAATTTAAAGCCACGACCGGTCTGTTCAAAGATTTCATCGATAAATGGACCTACATCAAAACGACCTCTGAAGGCGCGATTAAACAGCTGGCCAAACTGATGCTGAACAGCCTGTATGGCAAATTCGCCTCTAATCCGGATGTGACCGGTAAAGTTCCGTACCTGAAAGAAAATGGCGCACTGGGTTTTCGCCTGGGCGAAGAAGAAACGAAAGATCCGGTGTATACCCCGATGGGTGTTTTCATTACGGCCTGGGCACGTTACACGACCATCACCGCGGCCCAGGCATGCTATGATCGCATTATCTACTGTGATACCGATTCTATTCATCTGACGGGCACCGAAATCCCGGATGTGATTAAAGATATCGTTGATCCGAAAAAACTGGGTTATTGGGCCCACGAAAGTACGTTTAAACGTGCAAAATACCTGCGCCAGAAAACCTACATCCAGGATATCTACATGAAAGAAGTGGATGGCAAACTGGTTGAAGGTTCTCCGGATGATTACACCGATATCAAATTCAGTGTGAAATGCGCCGGCATGACGGATAAAATCAAAAAAGAAGTGACCTTCGAAAACTTCAAAGTTGGTTTCAGCCGCAAAATGAAACCGAAACCGGTGCAGGTTCCGGGCGGTGTGGTTCTGGTGGATGATACGTTTACCATTAAATCTGGCGGTAGTGCGTGGAGCCATCCGCAGTTCGAAAAAGGCGGTGGCTCTGGTGGCGGTTCTGGCGGTAGTGCCTGGAGCCACCCGCAGTTTGAAAAATAATAA

SEQ ID NO: 17 - protein, 319 aa, unidentified
  source 1..319: note = Bacillus cytotoxicus
MKTRIVSSVTTTLLLGSILMNPVAGAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 18 - protein, 336 aa, unidentified
  source 1..336: note = Bacillus cytotoxicus
MKRSKTYVKCLALSAVLASSALAMHTPVVAAQTTSQVVTDIGQNAKTHTSYNTFNNEQADNMTMSLKVTFIDDPSADKQIAVINTTGSFMKANPTLSDAPVDGYPIPGASVTLRYPSQYDIAMNLQDNTSRFFHVAPTNAVEETTVTSSVSYQLGGSIKASVTPSGPSGESGATGQVTWSDSVSYKQTSYKTNLIDQTNKHVKWNVFFNGYNNQNWGIYTRDSYHALYGNQLFMYSRTYPHETDARGNLVPMNDLPALTNSGFSPGMIAVVISEKDTEQSSIQVAYTKHADDYTLRPGFTFGTGNWVGNNIKDVDQKTFNKSFVLDWKNKKLVEKK

SEQ ID NO: 19 - DNA, 33 bp, synthetic construct
  source 1..33: mol_type = other DNA
gcaatcgtcgaatgcgtacgtcgtttttttttt

SEQ ID NO: 20 - DNA, 27 bp, synthetic construct
  source 1..27: mol_type = other DNA
  misc_feature 27: note = bicycle[6.1.0]nonyne (BCN) modification
aatgtacttcgttcagttacgtattgc

SEQ ID NO: 21 - protein, 10 aa, synthetic construct
GGSGYYSGSG

SEQ ID NO: 22 - DNA, 44 bp, synthetic construct
  source 1..44: mol_type = other DNA
  misc_feature 1: note = bicycle[6.1.0]nonyne (BCN)
aatgtacttcgttcagttacgtattgctgcttgggtgtttaacc

SEQ ID NO: 23 - DNA, 23 bp, synthetic construct
  source 1..23: mol_type = other DNA
cgacgtacgcattcgacgattgc

SEQ ID NO: 24 - DNA, 27 bp, synthetic construct
  source 1..27: mol_type = other DNA
gcaatacgtaactgaacgaagtacatt

SEQ ID NO: 25 - DNA, 44 bp, synthetic construct
  source 1..44: mol_type = other DNA
ggttaaacacccaagcagcaatacgtaactgaacgaagtacatt

SEQ ID NO: 26 - DNA, 18 bp, synthetic construct
  source 1..18: mol_type = other DNA
tttgaggcgagcggtcaa

SEQ ID NO: 27 - DNA, 33 bp, synthetic construct
  source 1..33: mol_type = other DNA
ggcgtctgcttgggtgtttaacctttttttttt

SEQ ID NO: 28 - DNA, 28 bp, synthetic construct
  source 1..28: mol_type = other DNA
aatgtacttcgttcagttacgtattgct

SEQ ID NO: 29 - DNA, 23 bp, synthetic construct
  source 1..23: mol_type = other DNA
ggttaaacacccaagcagacgcc

SEQ ID NO: 30 - DNA, 27 bp, synthetic construct
  source 1..27: mol_type = other DNA
gcaatacgtaactgaacgaagtacatt

SEQ ID NO: 31 - protein, 10 aa, synthetic construct
GGSGRRSGSG

SEQ ID NO: 32 - protein, 10 aa, synthetic construct
GGSGDDSGSG

(19) United States
(12) Patent Application Publication
(10) Pub. No.: US 2025/0188533 A1
(43) Pub. Date: Jun. 12, 2025

(54) NANOPORE

(71) Applicant: Oxford Nanopore Technologies PLC, Oxford (GB)

(72) Inventors: Elizabeth Jayne Wallace, Oxford (GB); Mark John Bruce, Oxford (GB)

(73) Assignee: Oxford Nanopore Technologies PLC, Oxford (GB)

(21) Appl. No.: 18/685,996

(22) PCT Filed: Aug. 26, 2022
(86) PCT No.: PCT/GB2022/052196

§ 371 Date: Feb. 23, 2024

(30) Foreign Application Priority Data
Aug. 26, 2021 (GB) 2112235.3

(51) Int. Cl. C12Q 1/6869; C07K 14/32; G01N 27/447; G01N 33/68

(52) U.S. Cl. CPC C12Q 1/6869; C07K 14/32; G01N 27/44791; G01N 33/6818

(57) ABSTRACT

The invention relates to mutant forms of Cytotoxin K. The invention also relates to methods of analyte detection and characterisation using Cytotoxin K, together with devices and kits for carrying out such methods.

### RELATED APPLICATIONS

This application is a national stage filing under 35 U.S.C. § 371 of international application number PCT/GB2022/052196, filed Aug. 26, 2022, which claims the benefit of United Kingdom application number GB 2112235.3, filed Aug. 26, 2021, each of which is herein incorporated by reference in its entirety.

### REFERENCE TO AN ELECTRONIC SEQUENCE LISTING

The contents of the electronic sequence listing (00366.70149US00-SEQ-KZM.xml; Size: 44,248 bytes; and Date of Creation: Jan. 16, 2024) are herein incorporated by reference in their entirety.

### FIELD

The invention relates to mutant forms of Cytotoxin K. The invention also relates to methods of analyte detection and characterisation using Cytotoxin K, together with devices and kits for carrying out such methods.

### BACKGROUND

Nanopore sensing is an approach to sensing that relies on the observation of individual binding or interaction events between analyte molecules and a detector. Nanopore sensors can be created by placing a single pore of nanometer dimensions in an insulating membrane and measuring voltage-driven ionic transport through the pore in the presence of analyte molecules. The identity of an analyte is revealed through its distinctive current signature, notably the duration and extent of current block and the variance of current levels. Such nanopore sensors are commercially available, such as the MinION™ device sold by Oxford Nanopore Technologies Ltd, comprising an array of nanopores integrated with an electronic chip.

There is currently a need for rapid and cheap nucleic acid (e.g. DNA or RNA) sequencing technologies across a wide range of applications. Existing technologies are slow and expensive mainly because they rely on amplification techniques to produce large volumes of nucleic acid and require a high quantity of specialist fluorescent chemicals for signal detection. Nanopore sensing has the potential to provide rapid and cheap nucleic acid sequencing by reducing the quantity of nucleotide and reagents required.

Furthermore, there is currently a need for new techniques to characterise polypeptides, especially at the single molecule level. Single molecule techniques for characterising biomolecules such as polynucleotides have proven to be particularly attractive due to their high fidelity and avoidance of amplification bias.

Whilst techniques to characterise (e.g. sequence) polynucleotides have been extensively developed, techniques to characterise polypeptides are less advanced, despite being of very significant biotechnological importance. For example, knowledge of a protein sequence can allow structure-activity relationships to be established and has implications in rational drug development strategies for developing ligands for specific receptors. Identification of post-translational modifications is also key to understanding the functional properties of many proteins. For example, typically 30-50% of protein species are phosphorylated in eukaryotes. Some proteins may have multiple phosphorylation sites, serving to activate or inactivate a protein, promote its degradation, or modulate interactions with protein partners. There is thus a pressing need for methods to characterise proteins and other polypeptides.

Known methods of characterising polypeptides include mass spectrometry and Edman degradation.

Protein mass spectrometry involves characterising whole proteins or fragments thereof in an ionised form. Known methods of protein mass spectrometry include electrospray ionisation (ESI) and matrix-assisted laser desorption/ionisation (MALDI). Mass spectrometry has some benefits, but results obtained can be affected by the presence of contaminants and it can be difficult to process fragile molecules without their fragmentation. Moreover, mass spectrometry is not a single molecule technique and provides only bulk information about the sample interrogated. Mass spectrometry is unsuitable for characterising differences within a population of polypeptide samples and is unwieldy when seeking to distinguish neighbouring residues.

Edman degradation is an alternative to mass spectrometry which allows the residue-by-residue sequencing of polypeptides. Edman degradation sequences polypeptides by sequentially cleaving the N-terminal amino acid and then characterising the individually cleaved residues using chromatography or electrophoresis. However, Edman sequencing is slow, involves the use of costly reagents, and like mass spectrometry is not a single molecule technique.

One attractive method of single molecule characterization of biomolecules such as polypeptides is nanopore sensing. Nanopore sensing is an approach to analyte detection and characterization that relies on the observation of individual binding or interaction events between the analyte molecules and an ion conducting channel. Nanopore sensors can be created by placing a single pore of nanometre dimensions in an electrically insulating membrane and measuring voltage-driven ion currents through the pore in the presence of analyte molecules. The presence of an analyte inside or near the nanopore will alter the ionic flow through the pore, resulting in altered ionic or electric currents being measured over the channel. The identity of an analyte is revealed through its distinctive current signature, notably the duration and extent of current blocks and the variance of current levels during its interaction time with the pore. Nanopore sensing has the potential to allow rapid and cheap polypeptide characterisation.

Nanopore sensing and characterisation of polypeptides has been proposed in the art, for example WO 2013/123379 and WO 2021/111125. However, there remains a need for alternative and/or improved methods of characterising polypeptides.

Two of the essential components of characterising analytes such as nucleic acids and amino acids using nanopore sensing are (1) the control of analyte movement through the pore and (2) the discrimination of analytes as analytes move through the pore. In the past, to achieve analyte discrimination the analyte has been passed through a mutant of hemolysin. This has provided current signatures that have been shown to be analyte dependent.

While the current range for analyte discrimination has been improved through mutation of the hemolysin pore, a new nanopore-based system would have higher performance if the current differences between analytes could be improved further. Furthermore, the provision of new and/or alternative system capable of use in the characterisation of polypeptide analytes would be of significant benefit to the proteomics field.

### SUMMARY

The disclosure relates to mutant Cytotoxin K monomers capable of forming a pore for use in methods for the characterisation of target analytes.

Accordingly, the invention provides a method of characterising a target analyte, comprising:
(a) contacting the target analyte with a pore comprising at least one mutant Cytotoxin K monomer comprising a variant of the amino acid sequence of SEQ ID NO: 1; such that the target analyte moves with respect to the pore;
   wherein the variant comprises one or more modifications at one or more positions in the region of SEQ ID NO: 1 between about S100 and about K170 which alter the ability of the monomer to interact with the analyte; and
(b) taking one or more measurements characteristic of the analyte as the analyte moves with respect to the pore,
thereby characterising the target analyte

The invention also provides a mutant Cytotoxin K monomer comprising a variant of the amino acid sequence of SEQ ID NO: 1; wherein the monomer is capable of forming a pore; and wherein the variant comprises one or more modifications at one or more positions in the region of SEQ ID NO: 1 between about S100 and about K170 which alter the ability of the monomer to interact with an analyte.

The invention also provides a construct comprising two or more covalently attached monomers derived from Cytotoxin K, wherein at least one of the monomers is a mutant Cytotoxin K monomer as defined according to the invention.

The invention also provides a polynucleotide which encodes a mutant Cytotoxin K monomer according to the invention or a construct according to the invention.

The invention also provides a homo-oligomeric pore comprising a plurality of mutant monomers according to the invention; wherein said pore is preferably a heptameric pore.

The invention also provides a hetero-oligomeric pore comprising at least one mutant monomer according to the invention; wherein said pore is preferably a heptameric pore.

The invention also provides a pore comprising at least one construct according to the invention.

The invention also provides a membrane comprising a pore according to the invention.

The invention also provides an array comprising a plurality of membranes according to the invention.

The invention also provides a device comprising the array of the invention, means for applying a potential across the membranes and means for detecting electrical or optical signals across the membranes.

The invention also provides a method of characterising a target analyte, comprising:
(a) contacting the target analyte with a pore according to the invention such that the target analyte moves with respect to the pore; and
(b) taking one or more measurements characteristic of the analyte as the analyte moves with respect to the pore,
thereby characterising the target analyte.

The invention also provides a use of a pore according to the invention to characterise a target analyte.

The invention also provides a method of characterising a target polypeptide, comprising:
(a) contacting the target polypeptide with a Cytotoxin K pore such that the target analyte moves with respect to the pore; and
(b) taking one or more measurements characteristic of the polypeptide as the polypeptide moves with respect to the pore,
thereby characterising the target polypeptide.

The invention also provides a use of a Cytotoxin K pore to characterise a target polypeptide.

The invention also provides a kit for characterising a target analyte comprising (a) a pore according to the invention and (b) a polynucleotide binding protein or polypeptide handling enzyme.

### BRIEF DESCRIPTION OF THE FIGURES

FIG. 1. Pairwise sequence alignment of CytK and aHL performed using Clustalx version 2.1. The transmembrane beta barrel of aHL is indicated by 3 boxes. Sp|P09616|HLA_STAAU is aHL and tr|A7GM18|A7GM18_BACCN is CytK.

FIG. 2. Structural model of the CytK pore. The model was made using the aHL structure as a template for CytK, where the structure of aHL was taken from the protein databank (accession code 7AHL). The Modeller software was used to make the CytK model. Top row shows the cartoon representation of the CytK model, whilst the bottom row shows the surface representation. The left-hand image of the bottom row shows the cross section through the pore.

FIG. 3. Predicted amino acid sequence of the CytK transmembrane beta barrel. The expected central regions of the 3 main constrictions are indicated by dashed boxes. Any residue with a number corresponds to residues that are predicted to point into the cavity of the pore. Any residue without a number corresponds to residues that are predicted to point towards the membrane.

FIG. 4. Comparison of the radial profiles of the CytK and aHL channels generated using the HOLE mapping software. The CytK model was made using the aHL structure as a template and the aHL structure was taken from the protein databank (accession code 7AHL).

FIG. 5. Ionic current profiles through aHL wild-type and CytK wild-type and mutants as the voltage is gradually increased in 25 mV steps every 30 seconds in both the negative and positive direction from (−)25 mV up to (−)200 mV. The applied voltage is shown by dashed lines (blue lines in original colour image), the raw current trace by grey lines (black lines in original colour image) and the event detected signal is shown by black lines (red lines in original colour image).

FIG. 6. Averaged ionic current profiles through aHL wild-type and CytK wild-type as the voltage is gradually increased in 25 mV steps every 30 seconds in both the negative and positive direction from (−)25 mV up to (−)200 mV. The top row shows the mean current within a voltage step grouped either by run (left) or pore batch (right). The bottom row shows the mean current of the first 100 ms within a voltage step grouped either by run (left) or pore batch (right). Plotting the mean current of the first 100 ms reduces the influence of pore gating into the measured current. Pore Batch A=aHL−(WT), Pore Batch B=CytK−(WT-H6), Pore Batch C=CytK−(WT-H6), Pore Batch D=CytK−(WT-H6-D8), Pore Batch E=CytK−(WT-H6-D8).

FIG. 7. Averaged ionic current profiles through CytK wild-type and CytK mutants as the voltage is gradually increased in 25 mV steps in both the negative and positive direction from (−)25 mV up to (−)200 mV. Panels 1 and 3 (top row in original image) show the mean current within a voltage step grouped either by run (panel 1) or pore batch (panel 3). Panels 2 and 4 (bottom row in original image) show the mean current of the first 100 ms within a voltage step grouped either by run (panel 2) or pore batch (panel 4). Plotting the mean current of the first 100 ms reduces the influence of pore gating into the measured current. Pore Batch B=CytK−(WT-H6), Pore Batch C=CytK−(WT-H6), Pore Batch D=CytK-(WT-H6-D8), Pore Batch E=CytK−(WT-H6-D8), Pore Batch F=CytK−(WT-E113S/K156S-D8), Pore Batch G=CytK−(WT-Q123S/Q146S-D8), Pore Batch H=CytK−(WT-K129S/E140S-D8), Pore Batch I=CytK−(WT-Q123S/Q146S/K129S/E140S-D8), Pore Batch J=CytK−(WT-Q123S/Q146S/K129S/E140S-D8), Pore Batch K=CytK−(WT-E113S/K156S/Q123S/Q146S/K129S/E140S), Pore Batch L=CytK−(WT-E113N/K156S/Q123S/Q146S/K129S/E140S-D8).

FIG. 8. Current versus time traces as DNA translocates through aHL wild-type and CytK wild-type and mutants. The raw current trace is shown by grey lines (black lines in original colour image) and the event detected signal is shown by black lines (red lines in original colour image). For each pore, the top row shows the full DNA current trace, the middle row shows the first section of the current trace and the bottom row shows a zoomed in view of the first section of the current trace.

FIG. 9. Table summarizing the pore characteristics of CytK wild-type and mutants. SNR is the signal to noise ratio which is the range of the signal divided by the noise as DNA is translocating through the pore. Median current is the median current of the signal as DNA is translocating through the pore.

FIG. 10. Box plots showing the pore characteristics of CytK wild-type and mutants. SNR is the signal to noise ratio which is the range of the signal divided by the noise as DNA is translocating through the pore. Median current is the median current of the signal as DNA is translocating through the pore.

FIG. 11. Bar charts showing the pore characteristic of CytK wild-type and mutants in condition 7, where condition 7 is 1 mM ATP, 10 mM MgCl2, 100 nM Hel308 mutant, 1 M NaCl, pH8, 100 mM HEPES, 10 mM Potassium Ferrocyanide, 10 mM Potassium Ferricyanide, 180 mV.

FIG. 12. Bar charts showing the pore characteristic of CytK wild-type and mutants in condition 9, where condition 9 is 1 mM ATP, 10 mM MgCl2, 100 nM Hel308 mutant, 625 mM KCl, pH8, 100 mM HEPES, 75 mM Potassium Ferrocyanide, 25 mM Potassium Ferricyanide.

FIG. 13. The polynucleotide-polypeptide conjugate used to translocate a peptide through a nanopore.

FIG. 14. Example current versus time traces as a polynucleotide-polypeptide conjugate translocates through CytK wild-type and mutants, where the polypeptide section comprises GGSGRRSGSG. The peptide section of the squiggles is highlighted by the boxes (red boxes in original colour image). The traces begin with a long flat section corresponding to the capture of the C3 leader on the adapter.

FIG. 15. Example current versus time traces as a polynucleotide-polypeptide conjugate translocates through the CytK mutant CytK-(WT-Q123S/Q146S/K129S/E140S), where the polypeptide section comprises either GGSGRRSGSG, GGSGYYSGSG or GGSGDDSGSG. The peptide section of the squiggles is highlighted by the boxes (red boxes in original colour image).

FIG. 16. The DNA sequencing Y-adapter used to translocate ssDNA through a nanopore.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

It should be appreciated that “embodiments” of the disclosure can be specifically combined together unless the context indicates otherwise. The specific combinations of all disclosed embodiments (unless implied otherwise by the context) are further disclosed embodiments of the claimed invention.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a helicase” includes two or more helicases, reference to “a monomer” refers to two or more monomers, reference to “a pore” includes two or more pores and the like.

All publications, patents and patent applications cited herein, whether supra or infra, are hereby incorporated by reference in their entirety.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4th ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide” and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypeptides that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” has at least 50%, 60%, 70%, 80%, 90%, 95% or 99% complete sequence identity to the amino acid sequence of the corresponding wild-type protein. Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

### Mutant Cytotoxin K Monomers

The invention provides methods of characterising an analyte using a pore comprising at least one mutant Cytotoxin K (CytK) monomer.

The invention also provides mutant Cytotoxin K (CytK) monomers. The mutant CytK monomers may be used to form pores of the invention. A mutant CytK monomer is a monomer whose sequence varies from that of a wild-type CytK monomer (SEQ ID NO: 1) and which retains the ability to form a pore. Methods for confirming the ability of mutant monomers to form pores are well-known in the art and are discussed in more detail below. For instance, the ability of a mutant monomer to form a pore can be determined as described in the Examples.

Pores comprising the mutant monomers of the invention have an increased current range when subject to an applied potential in a nanopore-based method of analyte characterisation, relative to a pore consisting of wild type CytK monomers. An increased current range makes it easier to identify and characterise target analytes, and in particular makes it easier to discriminate between components of the target analyte. For example, when the target analyte is a polypeptide, an increased current range makes it easier to discriminate between amino acids in the polypeptide.

Pores comprising a mutant CytK monomer of the invention may be used to characterise any suitable analyte. Suitable analytes are described further herein. The increased current range in particular render the pores comprising a mutant CytK monomer of the invention particularly applicable to nanopore-based methods of characterising polypeptide analytes as described herein. Techniques to characterise polypeptides are of significant biotechnological importance. For example, knowledge of a protein sequence can allow structure-activity relationships to be established and has implications in rational drug development strategies for developing ligands for specific receptors. Identification of post-translational modifications is also key to understanding the functional properties of many proteins. For example, typically 30-50% of protein species are phosphorylated in eukaryotes. Some proteins may have multiple phosphorylation sites, serving to activate or inactivate a protein, promote its degradation, or modulate interactions with protein partners. Described herein is the successful utilisation of pores comprising a mutant CytK monomer in a nanopore based method of characterising a target polypeptide. Accordingly, the inventors have surprisingly identified a novel means for characterising polypeptide analytes.

The inventors have surprisingly identified a region within the CytK monomer which can be modified to alter the interaction between the monomer and an analyte, such as when the analyte is characterised using nanopore-based methods of analyte characterisation described herein comprising the use of a pore comprising a CytK mutant monomer of the invention. With reference to the wild type polypeptide sequence of a CytK monomer as defined by SEQ ID NO: 1, the region is from about position S100 to about position K170 in SEQ ID NO: 1. At least a part of this region typically contributes to the membrane spanning region of CytK. At least a part of this region typically contributes to the barrel or channel of CytK. At least a part of this region typically contributes to the internal wall or lining of CytK.

The improved analyte characterisation properties of the CytK mutant monomers are achieved via the introduction of one or more modification at one or more positions in the region of SEQ ID NO: 1 between about S100 and about K170 which alter the ability of the monomer to interact with the analyte. Preferable mutations are further described herein. Accordingly, provided is a mutant CytK monomer comprising a variant of the amino acid sequence of SEQ ID NO: 1; wherein the monomer is capable of forming a pore; and wherein the variant comprises one or more modifications at one or more positions in the region of SEQ ID NO 1: between about S100 and about K170 which alter the ability of the monomer to interact with an analyte.

In accordance with the invention, the variant comprises one or more modifications at one or more positions in the region of SEQ ID NO: 1 between about S100 and K170 which alter the ability of the monomer, or preferably the region, to interact with an analyte. The interaction between the monomer and the analyte may be increased or decreased. An increased interaction between the monomer and an analyte will, for example, facilitate capture of the analyte by pores comprising the mutant monomer. A decreased interaction between the monomer and an analyte will, for example, improve recognition or discrimination of the analyte. Recognition or discrimination of the analyte may be improved by increasing the current range by virtue of the modifications to the CytK monomer between about S100 and K170 of SEQ ID NO: 1 described herein. The improved recognition or discrimination of the analyte may particularly be improved achieved via five main mechanisms, namely by independent changes in the:
sterics (e.g. increasing or decreasing the size of amino acid residues);
net charge of the amino acid residue at the modified position (e.g. introducing or removing negative (−ve) charge and/or introducing or removing positive (+ve) charge);
hydrogen bonding characteristics of the amino acid residue at the modified position (e.g. introducing amino acids that can hydrogen bond to the analyte);
pi stacking (e.g. introduce to or remove from the amino acid residue at the modified position one or more chemical groups that interact through delocalized electron pi systems); and/or
amino acid residue at the modified position, thereby changing the structure of the pore (e.g. introducing amino acids that increase or decrease the size of the barrel or channel).

Thus, the one or more modification may each independently (a) alter the size of the amino acid residue at the modified position; (b) alter the net charge of the amino acid residue at the modified position; (c) alter the hydrogen bonding characteristics of the amino acid residue at the modified position; (d) introduce to or remove from the amino acid residue at the modified position one or more chemical groups that interact through delocalized electron pi systems and/or (e) alter the structure of the amino acid residue at the modified position.

Any one or more of these mechanisms of independent alteration may be responsible for the improved properties of the pores formed from the mutant monomers of the invention. For instance, a pore comprising a mutant monomer of the invention may display improved polypeptide and/or polynucleotide reading properties as a result of altered sterics, altered hydrogen bonding and an altered structure.

Accordingly, provided herein is a method of characterising a target analyte, comprising:
(a) contacting the target analyte with a pore comprising at least one mutant Cytotoxin K monomer comprising a variant of the amino acid sequence of SEQ ID NO: 1; such that the target analyte moves with respect to the pore;
   wherein the variant comprises one or more modifications at one or more positions in the region of SEQ ID NO: 1 between about S100 and about K170 which alter the ability of the monomer to interact with the analyte; and
(b) taking one or more measurements characteristic of the analyte as the analyte moves with respect to the pore,
thereby characterising the target analyte.

Also provided is a mutant CytK monomer comprising a variant of the amino acid sequence of SEQ ID NO: 1; wherein the monomer is capable of forming a pore; and wherein the variant comprises one or more modifications at one or more positions in the region of SEQ ID NO 1: between about S100 and about K170 which alter the ability of the monomer to interact with an analyte.

The ability of the monomer to interact with a target analyte to interact with an analyte can be determined using methods that are well-known in the art. The monomer may interact with an analyte in any way, e.g. by non-covalent interactions, such as hydrophobic interactions, hydrogen bonding, Van der Waal's forces, pi(π)-cation interactions or electrostatic forces. For instance, the ability of the region to bind to an analyte can be measured using a conventional binding assay. Suitable assays include, but are not limited to, fluorescence-based binding assays, nuclear magnetic resonance (NMR), Isothermal Titration calorimetry (ITC) or Electron spin resonance (ESR) spectroscopy. Alternatively, the ability of a pore comprising one or more of the mutant monomers to interact with an analyte can be determined using any of the methods discussed above or below. Preferred assays are described in the Examples.

The one or more modifications are within the region from about position 100 to about position 170 of SEQ ID NO: 1. The one or more modifications are preferably within the region from about position 110 to about position 160 of SEQ ID NO: 1. The one or more modifications are yet more preferably within the region from about position 113 to about position 156 of SEQ ID NO: 1.

Modifications of protein nanopores that alter their ability to interact with an analyte, and in particular improve their current range, are well documented in the art. For instance, such modifications are disclosed in WO 2010/034018, WO 2010/055307, WO 2013/153359 and WO 2016/034591. Similar modifications can be made to the CytK monomer in accordance with this invention.

Any number of modifications may be made, such as 1, 2, 5, 10, 15, 20, 30 or more modifications. Any modification(s) can be made as long as the ability of the monomer to interact with a polynucleotide is altered and the monomer remains capable of forming a pore. Suitable modifications include, but are not limited to, amino acid substitutions, amino acid additions and amino acid deletions. The one or more modifications are preferably one or more substitutions. This is discussed in more detail below.

The one or more modifications preferably (a) alter the steric effect of the monomer, or preferably alter the steric effect of the region, (b) alter the net charge of the monomer, or preferably alter the net charge of the region, (c) alter the ability of the monomer, or preferably of the region, to hydrogen bond with the analyte, (d) introduce or remove chemical groups that interact through delocalized electron pi systems and/or (e) alter the structure of the monomer, or preferably alter the structure of the region. The one or more modifications more preferably result in any combination of (a) to (e), such as (a) and (b); (a) and (c); (a) and (d); (a) and (e); (b) and (c); (b) and (d); (b) and (e); (c) and (d); (c) and (e); (d) and (e), (a), (b) and (c); (a), (b) and (d); (a), (b) and (e); (a), (c) and (d); (a), (c) and (e); (a), (d) and (e); (b), (c) and (d); (b), (c) and (e); (b), (d) and (e); (c), (d) and (e); (a), (b), (c) and d); (a), (b), (c) and (e); (a), (b), (d) and (e); (a), (c), (d) and (e); (b), (c), (d) and (e); and (a), (b), (c) and (d).

For (a), the steric effect of the monomer can be increased or decreased. Any method of altering the steric effects may be used in accordance with the invention. The introduction of bulky residues, such as phenylalanine (F), tryptophan (W), tyrosine (Y) or histidine (H), increases the sterics of the monomer. The one or more modifications are preferably the introduction of one or more of F, W, Y and H. Any combination of F, W, Y and H may be introduced. The one or more of F, W, Y and H may be introduced by addition. The one or more of F, W, Y and H are preferably introduced by substitution. Suitable positions for the introduction of such residues are discussed in more detail below.

The removal of bulky residues, such as phenylalanine (F), tryptophan (W), tyrosine (Y) or histidine (H), conversely decreases the sterics of the monomer. The one or more modifications are preferably the removal of one or more of F, W, Y and H. Any combination of F, W, Y and H may be removed. The one or more of F, W, Y and H may be removed by deletion. The one or more of F, W, Y and H are preferably removed by substitution with residues having smaller side groups, such as serine(S), threonine (T), alanine (A) and valine (V).

For (b), the net charge can be altered in any way. The net positive charge is preferably increased or decreased. The net positive charge can be increased in any manner. The net positive charge is preferably increased by introducing, preferably by substitution, one or more positively charged amino acids and/or neutralising, preferably by substitution, one or more negative charges.

The net positive charge is preferably increased by introducing one or more positively charged amino acids. The one or more positively charged amino acids may be introduced by addition. The one or more positively charged amino acids are preferably introduced by substitution. A positively charged amino acid is an amino acid with a net positive charge. The positively charged amino acid(s) can be naturally-occurring or non-naturally-occurring. The positively charged amino acids may be synthetic or modified. For instance, modified amino acids with a net positive charge may be specifically designed for use in the invention. A number of different types of modification to amino acids are well known in the art. The one or more modifications comprising the introduction of one or more positively charged amino acids preferably comprise the introduction of one or more of histidine (H), lysine (K) and arginine (R) by way of substitution or addition, although most preferably by substitution. Suitable positions for the introduction of such residues are discussed in more detail below.

Methods for adding or substituting naturally-occurring amino acids are well known in the art. For instance, the nucleotides which constitute a codon that are comprised within a polynucleotide coding sequence may be modified such that the nucleotide contents of the codon is altered, thereby leading to a different amino acid to be coded for by said codon. Such a polynucleotide may then be expressed as discussed below.

Methods for adding or substituting non-naturally-occurring amino acids are also well known in the art. For instance, non-naturally-occurring amino acids may be introduced by including synthetic aminoacyl-tRNAs in the IVTT system used to express the pore. Alternatively, they may be introduced by expressing the monomer in E. coli that are auxotrophic for specific amino acids in the presence of synthetic (i.e. non-naturally-occurring) analogues of those specific amino acids. They may also be produced by naked ligation if the pore is produced using partial peptide synthesis.

In the one or more modifications, any amino acid may be substituted with a positively charged amino acid. In the one or more modifications, one or more uncharged amino acids, non-polar amino acids and/or aromatic amino acids may be substituted with one or more positively charged amino acids. Uncharged amino acids have no net charge. Suitable uncharged amino acids include, but are not limited to, cysteine (C), serine(S), threonine (T), methionine (M), asparagine (N) and glutamine (Q). Non-polar amino acids have non-polar side chains. Suitable non-polar amino acids include, but are not limited to, glycine (G), alanine (A), proline (P), isoleucine (I), leucine (L) and valine (V). Aromatic amino acids have an aromatic side chain. Suitable aromatic amino acids include, but are not limited to, histidine (H), phenylalanine (F), tryptophan (W) and tyrosine (Y). Preferably, in the one or more modifications, one or more negatively charged amino acids are substituted with one or more positively charged amino acids. Suitable negatively charged amino acids include, but are not limited to, aspartic acid (D) and glutamic acid (E).

In the one or more modifications, preferred introductions include, but are not limited to, substitution of E with K, M with R, substitution of M with H, substitution of M with K, substitution of D with R, substitution of D with H, substitution of D with K, substitution of E with R, substitution of E with H, substitution of N with R, substitution of T with R and substitution of G with R. Most preferably E is substituted with K.

In the one or more modifications, any number of positively charged amino acids may be introduced or substituted. For instance, 1, 2, 5, 10, 15, 20, 25, 30 or more positively charged amino acids may be introduced or substituted.

The net positive charge is more preferably increased by neutralising one or more negative charges. The one or more negative charges may be neutralised by substituting one or more negatively charged amino acids with one or more uncharged amino acids, non-polar amino acids and/or aromatic amino acids. The removal of negative charge increases the net positive charge. The uncharged amino acids, non-polar amino acids and/or aromatic amino acids can be naturally-occurring or non-naturally-occurring. They may be synthetic or modified. Suitable uncharged amino acids, non-polar amino acids and aromatic amino acids are discussed above. Preferred substitutions include, but are not limited to, substitution of E with Q, substitution of E with S, substitution of E with A, substitution of D with Q, substitution of E with N, substitution of D with N, substitution of D with G and substitution of D with S.

Any number and combination of uncharged amino acids, non-polar amino acids and/or aromatic amino acids may substituted in the one or more modifications. For instance, 1, 2, 5, 10, 15, 20, 25, or 30 or more uncharged amino acids, non-polar amino acids and/or aromatic amino acids may be substituted. Negatively charged amino acids may be substituted with (1) uncharged amino acids; (2) non-polar amino acids; (3) aromatic amino acids; (4) uncharged amino acids and non-polar amino acids; (5) uncharged amino acids and aromatic amino acids; and (5) non-polar amino acids and aromatic amino acids; or (6) uncharged amino acids, non-polar amino acids and aromatic amino acids.

The one or more negative charges may be neutralised by introducing one or more positively charged amino acids near to, such as within 1, 2, 3 or 4 amino acids, or adjacent to one or more negatively charged amino acids. Examples of positively and negatively charged amino acids are discussed above. The positively charged amino acids may be introduced in any manner discussed above, for instance by substitution.

The net positive charge is preferably decreased by introducing one or more negatively charged amino acids and/or neutralising one or more positive charges. Ways in which this might be done will be clear from the discussion above with reference to increasing the net positive charge. All of the embodiments discussed above with reference to increasing the net positive charge equally apply to decreasing the net positive charge except the charge is altered in the opposite way. In particular, the one or more positive charges are preferably neutralised by substituting one or more positively charged amino acids with one or more uncharged amino acids, non-polar amino acids and/or aromatic amino acids or by introducing one or more negatively charged amino acids near to, such as within 1, 2, 3 or 4 amino acids of, or adjacent to one or more negatively charged amino acids.

The net negative charge is preferably increased or decreased. All of the above embodiments discussed above with reference to increasing or decreasing the net positive charge equally apply to decreasing or increasing the net negative charge respectively.

For (c), the ability of the monomer to hydrogen bond may be altered in any suitable manner. For example, the one or more modifications may comprise the introduction of one or more of serine(S), threonine (T), asparagine (N), glutamine (Q), tyrosine (Y) or histidine (H) by addition or substitution, thereby increasing the hydrogen bonding ability of the monomer. The one or more modifications preferably comprise the introduction of one or more of S, T, N, Q, Y and H in any suitable combination, preferably wherein the introduction is by substitution. Suitable positions for the introduction of such residues are discussed in more detail below.

The removal of serine(S), threonine (T), asparagine (N), glutamine (Q), tyrosine (Y) or histidine (H) decreases the hydrogen bonding ability of the monomer. For example, the one or more modifications may comprise the removal of one or more of S, T, N, Q, Y and H. The one or more modifications preferably comprise the removal of any combination of S, T, N, Q, Y and H by deletion or by substitution in any suitable combination, thereby decreasing the hydrogen bonding ability of the monomer. The one or more modifications preferably comprise the substitution with other amino acids which hydrogen bond less well, such as alanine (A), valine (V), isoleucine (I) and leucine (L).

For (d), the introduction of aromatic residues, such as phenylalanine (F), tryptophan (W), tyrosine (Y) or histidine (H), also increases the pi stacking in the monomer. The removal of aromatic residues, such as phenylalanine (F), tryptophan (W), tyrosine (Y) or histidine (H), also increases the pi stacking in the monomer. Such amino acids can be introduced or removed as discussed above with reference to (a).

For (e), one or more modifications made in accordance with the invention which alter the structure of the monomer. For example, one or more loop regions can be removed, shortened or extended. This typically facilitates the entry or exit of a polynucleotide into or out of the pore. The one or more loop regions may be the cis side of the pore, the trans side of the pore or on both sides of the pore. Alternatively, one or more regions of the amino terminus and/or the carboxy terminus of the pore can be extended or deleted. This typically alters the size and/or charge of the pore.

It will be clear from the discussion above that the introduction of certain amino acids will enhance the ability of the monomer to interact with an analyte via more than one mechanism. For instance, the substitution of E with H will not only increase the net positive charge (by neutralising negative charge) in accordance with (b), but will also increase the ability of the monomer to hydrogen bond in accordance with (c).

The inventors surprisingly identified three constrictions in a pore consisting of wild type CytK monomers. A constriction is typically a narrowing in the channel which runs through the nanopore which may determine or control the signal obtained in any of the known nanopore-based methods of analyte characterisation, or any methods of analyte characterisation described herein, when the analyte moves with respect to the nanopore. The structure of each CytK monomer in the pore leads to the formation of the three constrictions in the barrel region of the pore. The amino acids responsible for the formation of the three constrictions are comprised between about S100 and K170.

Accordingly, the mutant CytK monomer of the invention may comprise one or modifications at one or more positions in the region of SEQ ID NO: 1 between about S100 and K170 which alter the ability of the monomer to interact with an analyte, wherein the modifications alter one or more of the three constrictions in a pore comprising a CytK monomer of the invention relative to a pore consisting of wild type CytK monomers. Said modifications may therefore alter the interaction of the constriction with an analyte as the analyte moves through the pore. Preferably, the monomer of the invention is capable of forming a pore having a solvent-accessible channel from a first opening to a second opening of said pore; the solvent-accessible channel comprising at least one constriction; and wherein the one or more modifications are made to amino acids in said constriction. Thus, by modifying the region of a CytK monomer which is responsible for forming the three constrictions in a wild type CytK pore, the interaction between a CytK monomer and an analyte, such as when the analyte is characterised using nanopore-based methods of analyte characterisation described herein, can be altered.

The amino acids responsible for the formation of the three constrictions are comprised between about S100 and K170 of SEQ ID NO:1 defining a CytK monomer, and preferably face inwards into the channel region when said CytK monomer assembles to form a CytK pore. Preferably, therefore, the one or more modifications which alter the characteristics of the constriction region of the CytK monomer of the invention relative to a wild type CytK monomer are made to amino acids which face inwards said CytK monomer assembles to form a CytK pore. The amino acids responsible for the contribution of a single CytK monomer to a constriction in a CytK pore typically comprise a pair of amino acids in the CytK monomer. Accordingly, the one or more modifications to the amino acids responsible for the formation of the three constrictions are comprised between about S100 and K170 of SEQ ID NO:1 and is preferably a modification to a pair of amino acids. Such pairs of amino acids are described further herein.

Thus, the one or more modification that alter the constriction may each independently (a) alter the size of the constriction (e.g. by increasing or decreasing the size of the amino acid residue at the modified position); (b) alter the net charge of the constriction (e.g. by altering the net charge of the amino acid residue at the modified position); (c) alter the hydrogen bonding characteristics of the amino acid residues in the constriction (e.g. by altering the hydrogen bonding characteristics of the amino acid residue at the modified position); (d) introduce to or remove from the constriction one or more chemical groups that interact through delocalized electron pi systems (e.g. by introducing to or remove from the amino acid residue at the modified position one or more chemical groups that interact through delocalized electron pi systems); and/or (e) alter the structure of the constriction (e.g. by altering the structure of the amino acid residue at the modified position). The one or more modifications which alter (a)-(e) with respect to the constriction may be identical to those described herein with respect to the monomer generally.

As described herein (see particularly the Examples), the inventors have identified three constrictions in a wild type CytK pore (i.e. a CytK pore consisting of wild type CytK monomers). The loop region of each CytK monomer comprises amino acids which define the three constrictions of a wild type CytK pore. Each constriction is defined by amino acids on opposite sides of the loop region. The upper constriction (closest to the cap region of the pore) is preferably defined by the region of SEQ ID NO: 1 between about X109 and about T117, more preferably between V111 and T115, and between about S152 and about X160, preferably between S154 and X158. The lower constriction (furthest from the cap region of the pore) is preferably defined by the region of SEQ ID NO: 1 between about G126 and about V132, preferably between S127 and S131, and between about P137 and about A143, preferably between S138 and G142. The middle constriction (furthest from the cap region of the pore) is preferably defined by the region of SEQ ID NO: 1 between about S119 and about G126, preferably between S121 and G125, and between about A143 and about S150, preferably between T144 and T148.

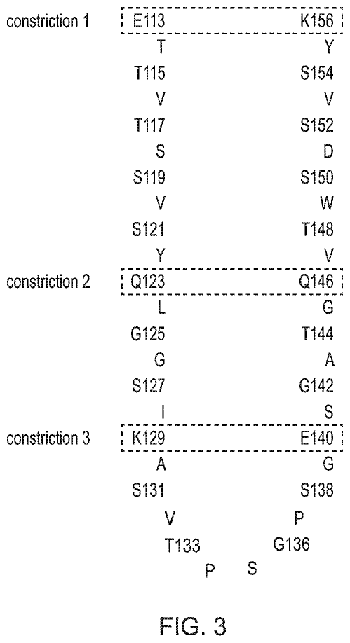

In wild type CytK, amino acids from about V111 to about S131 of SEQ ID NO: 1 and from about S138 to about T158 of SEQ ID NO: 1 form a loop region of the pore which comprises the three constrictions (see FIG. 3). Preferably, the amino acids that form the three constrictions comprises amino acids in the loop region that face inwards into the channel of the pore. More preferably, an amino acid between about V111 to about S131 of SEQ ID NO: 1 forms a pair with an amino acid between S138 to about T158 of SEQ ID NO: 1 in order to form a constriction in the channel of the pore. Each amino acid in a pair within a pair is on the opposite side of the loop region to one another. Accordingly, in a monomer of the invention described herein, the variant may comprise one or more modifications in the region of SEQ ID NO: 1 between about V111 and about S131; and/or between about S135 and about T158. Preferably, in a monomer of the invention described herein, the variant may comprise one or more modifications in the region of SEQ ID NO: 1 between about V111 and about S131 and between about S135 and about T158. In another aspect, in a monomer of the invention described herein, the variant may comprise 1, 2, 3, 4, 5, 6, 7, 8, 9, or 10 or more modifications between about V111 and about S131 in SEQ ID NO: 1; and 1, 2, 3, 4, 5, 6, 7, 8, 9, or 10 or more modifications between about S135 and about T158 in SEQ ID NO:1. Most preferably, the same number of modifications are made in the region of SEQ ID NO: 1 between about V111 and about S131 and between about S135 and about T158.

In the CytK monomer of the invention, the variant may comprise one or more modifications in the region of SEQ ID NO: 1 between about S119 and about G126, preferably between S121 and G125; and/or between about A143 and about S150, preferably between T144 and T148. Preferably, in a monomer of the invention described herein, the variant may comprise one or more modifications in the region of SEQ ID NO: 1 between about S119 and about G126, preferably between S121 and G125, and between about A143 and about S150, preferably between T144 and T148. In another aspect, in a monomer of the invention described herein, the variant may comprise 1, 2, 3, 4, or 5 or more modifications between about S119 and about G126 of SEQ ID NO: 1, preferably between S121 and G125; and 1, 2, 3, 4 or 5 or more modifications between about A143 and about S150 of SEQ ID NO: 1, preferably between T144 and T148. Most preferably, the same number of modifications are made in the region of SEQ ID NO: 1 between about S119 and about G126 of SEQ ID NO: 1, preferably between S121 and G125, and between about A143 and about S150 of SEQ ID NO: 1, preferably between T144 and T148.

In the CytK monomer of the invention, the variant may comprise one or more modifications in the region of SEQ ID NO: 1 between about G126 and about V132, preferably between S127 and S131; and/or between about P137 and about A143, preferably between S138 and G142. Preferably, in a monomer of the invention described herein, the variant may comprise one or more modifications in the region of SEQ ID NO: 1 between about G126 and about V132, preferably between S127 and S131, and between about P137 and about A143, preferably between S138 and G142. In another aspect, in a monomer of the invention described herein, the variant may comprise 1, 2, 3, 4, or 5 or more modifications between about G126 and about V132 of SEQ ID NO: 1, preferably between S127 and S131; and 1, 2, 3, 4 or 5 or more modifications between about P137 and about A143 of SEQ ID NO: 1, preferably between S138 and G142. Most preferably, the same number of modifications are made in the region of SEQ ID NO: 1 between about G126 and about V132 of SEQ ID NO: 1, preferably between S127 and S131, and between about P137 and about A143 of SEQ ID NO: 1, preferably between S138 and G142.

In the CytK monomer of the invention, the variant may comprise one or more modifications in the region of SEQ ID NO: 1 between about N109 and about T117, preferably between V111 and T115; and/or between about S152 and about Y160, preferably between S154 and T158. Preferably, in a monomer of the invention described herein, the variant may comprise one or more modifications in the region of SEQ ID NO: 1 between about N109 and about T117, preferably between V111 and T115, and between about S152 and about Y160, preferably between S154 and T158. In another aspect, in a monomer of the invention described herein, the variant may comprise 1, 2, 3, 4, or 5 or more modifications between about N109 and about T117 of SEQ ID NO: 1, preferably between V111 and T115; and 1, 2, 3, 4 or 5 or more modifications between about S152 and about Y160 of SEQ ID NO: 1, preferably between S154 and T158. Most preferably, the same number of modifications are made in the region of SEQ ID NO: 1 between about N109 and about T117 of SEQ ID NO: 1, preferably between V111 and T115, and between about S152 and about Y160 of SEQ ID NO: 1, preferably between S154 and T158.

The variant preferably comprises a modification at one or more of the following positions of SEQ ID NO: 1: E113, T115, T117, S119, S121, Q123, G125, S127, K129, S131, V132, T133, P134, S135, G136, P137, S138, E140, G142, T144, Q146, T148, S150, S152, S154 and K156. The variant preferably comprises modification at 1, 2, 3, 4, 5, 6, 7, 8, 9, 10, 11, 12, 13, 14, 15, 16, 17, 18, 19, 20, 21, 22, 23, 24, or 25 or more of these positions. The variant may independently comprise one or more amino acid substitutions, additions and/or deletions at said one or more positions. The amino acids substituted into the variant may be naturally-occurring or non-naturally occurring derivatives thereof. The amino acids substituted into the variant may be D-amino acids. In particular, the variant may comprise one or more amino acid substitutions at the positions listed above, and the amino acid(s) substituted into the variant are selected from aspartate, glutamate, serine, threonine, asparagine, glutamine, glycine, alanine, valine, leucine, isoleucine, cysteine, arginine, lysine and phenylalanine.

The variant preferably comprises one or more of the following modifications of SEQ ID NO: 1:
a) E113S/T/N/Q/G/A/V/L/I/C/R/K/F/Y;
b) T115S/N/Q/G/A/V/L/I/C/R/K/F;
c) T117S/N/Q/G/A/V/L/I/C/R/K/F;
d) S119T/N/Q/G/A/V/L/I/C/R/K/F;
e) S121T/N/Q/G/A/V/L/I/C/R/K/F;
f) Q123S/T/N/G/A/V/L/I/C/R/K/F/M/Y;
g) G125S/T/N/Q/A/V/L/I/C/R/K/F;
h) S127T/N/Q/G/A/V/L/I/C/R/K/F;
i) K129S/T/N/Q/G/A/V/L/I/C/R/F/Y;
j) S131T/N/Q/G/A/V/L/I/C/R/K/F;
k) V132S/T/N/Q/G/A/L/I/C/R/K/F;
l) T133S/N/Q/G/A/V/L/I/C/R/K/F;
m) P134S/T/N/Q/G/A/V/L/I/C/R/K/F;
n) S135T/N/Q/G/A/V/L/I/C/R/K/F;
o) G136S/T/N/Q/A/V/L/I/C/R/K/F;
p) P137S/T/N/Q/G/A/V/L/I/C/R/K/F;
q) S138T/N/Q/G/A/V/L/I/C/R/K/F;
r) E140S/T/N/Q/G/A/V/L/I/C/R/K/F;
s) G142S/T/N/Q/A/V/L/I/C/R/K/F;
t) T144S/N/Q/G/A/V/L/I/C/R/K/F;
u) Q146S/T/N/G/A/V/L/I/C/R/K/F/M/Y;
v) T148S/N/Q/G/A/V/L/I/C/R/K/F;
w) S150T/N/Q/G/A/V/L/I/C/R/K/F;
x) S152T/N/Q/G/A/V/L/I/C/R/K/F;
y) S154T/N/Q/G/A/V/L/I/C/R/K/F; and
z) K156S/T/N/Q/G/A/V/L/I/C/R/F.

The inventors have particularly identified six amino acids, forming three pairs, in the loop region of wild type CytK which are considered to be amino acids which are responsible for the three constrictions in a wild type CytK pore. Accordingly, the variant may comprise a modification at any one or more the six amino acids as follows:
a) E113;
b) Q123;
c) K129;
d) E140;
e) Q146; and
f) K156.

The variant may particularly comprise modifications in SEQ ID NO: 1 at Q123 and/or Q146. The variant may particularly comprise modification at Q123 and Q146 in SEQ ID NO: 1.

The variant may particularly comprise modifications in SEQ ID NO: 1 at K129 and/or E140. The variant may particularly comprise modification at K129 and E140 in SEQ ID NO: 1.

The variant may particularly comprise modifications in SEQ ID NO: 1 at E113 and/or K156. The variant may particularly comprise modification at E113 and K156 in SEQ ID NO: 1.

The variant may comprise one or more modifications within two or three of the constrictions of CytK. Accordingly, the variant may comprise modifications in SEQ ID NO: 1 at:
(i) Q123 and/or Q146; and (ii) K129 and/or E140.
(i) E113 and/or K156; and (ii) Q123 and/or Q146; or
(i) E113 and/or K156; and (ii) K129 and/or E140.

More preferably, the variant may comprise one or more modifications within the middle and lower constriction. Accordingly, the variant may comprise modifications at Q123 and/or Q146; and (ii) K129 and/or E140 in SEQ ID NO: 1, and even more preferably modifications at all of Q123, Q146, K129 and E140.

The variant may comprise one or more of the following modification in SEQ ID NO: 1:
a) E113S/N/Y/K/R;
b) Q123S/A/N/M/Y/G/K/R;
c) K129S/N/Y;
d) E140S/N/K/R;
e) Q146S/A/N/M/K/R/G/Y; and
f) K156S/N.

The variant may comprise any of the following modification pairs in SEQ ID NO: 1:
a) E113S/T/N/Q/G/A/V/L/I/C/R/K/F and K156S/T/N/Q/G/A/V/L/I/C/R/F;
b) Q123S/T/N/G/A/V/L/I/C/R/K/F and Q146S/T/N/G/A/V/L/I/C/R/K/F; or
c) K129S/T/N/Q/G/A/V/L/I/C/R/F and E140S/T/N/Q/G/A/V/L/I/C/R/K/F.

The variant even more preferably may comprise any of the following two or more pairs of mutations in SEQ ID NO:1:
a) E113S/T/N/Q/G/A/V/L/I/C/R/K/F and K156S/T/N/Q/G/A/V/L/I/C/R/F and Q123S/T/N/G/A/V/L/I/C/R/K/F and Q146S/T/N/G/A/V/L/I/C/R/K/F;
b) E113S/T/N/Q/G/A/V/L/I/C/R/K/F and K156S/T/N/Q/G/A/V/L/I/C/R/F and K129S/T/N/Q/G/A/V/L/I/C/R/F and E140S/T/N/Q/G/A/V/L/I/C/R/K/F;
c) Q123S/T/N/G/A/V/L/I/C/R/K/F and Q146S/T/N/G/A/V/L/I/C/R/K/F and K129S/T/N/Q/G/A/V/L/I/C/R/F and E140S/T/N/Q/G/A/V/L/I/C/R/K/F; or
d) E113S/T/N/Q/G/A/V/L/I/C/R/K/F and K156S/T/N/Q/G/A/V/L/I/C/R/F and Q123S/T/N/G/A/V/L/I/C/R/K/F and Q146S/T/N/G/A/V/L/I/C/R/K/F and K129S/T/N/Q/G/A/V/L/I/C/R/F and E140S/T/N/Q/G/A/V/L/I/C/R/K/F.

The monomer of the invention may particularly comprise a variant of the sequence of SEQ ID NO: 1, wherein the variant comprises the following modifications:
a) E113S and K156S;
b) Q123S and Q146S;
c) K129S and E140S;
d) Q123S, Q146S, K129S and E140S; or
e) E113S, K156S, Q123S, Q146S, K129S and E140S.

In addition to the specific mutations discussed above, the variant may include other mutations. Over the entire length of the amino acid sequence of SEQ ID NO: 1, a variant will preferably be at least 50% homologous to that sequence based on amino acid identity. More preferably, the variant may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 1 over the entire sequence. There may be at least 80%, for example at least 85%, 90% or 95%, amino acid identity over a stretch of 100 or more, for example 125, 150, 175 or 200 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

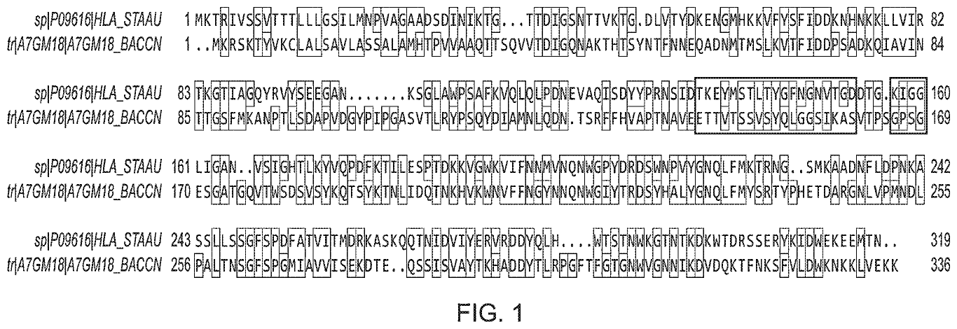

The mutant monomer of the invention may be chemically modified. In particular, the monomer may be chemically modified in any way and at any site. The mutant monomer is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. Suitable non-natural amino acids include, but are not limited to, 4-azido-L-phenylalanine (Faz) and any one of the amino acids numbered 1-71 in FIG. 1 of Liu C. C. and Schultz P. G., Annu. Rev. Biochem., 2010, 79, 413-444. The mutant monomer may be chemically modified by the attachment of any molecule. For instance, the mutant monomer may be chemically modified by attachment of a polyethylene glycol (PEG), a nucleic acid, such as DNA, a dye, a fluorophore or a chromophore.

In some embodiments, the mutant monomer is chemically modified with a molecular adaptor that facilitates the interaction between a pore comprising the monomer and a target analyte, a target nucleotide or target polynucleotide. The presence of the adaptor improves the host-guest chemistry of the pore and the nucleotide or polynucleotide and thereby improves the sequencing ability of pores formed from the mutant monomer. The principles of host-guest chemistry are well-known in the art. The adaptor has an effect on the physical or chemical properties of the pore that improves its interaction with the nucleotide or polynucleotide. The adaptor may alter the charge of the barrel or channel of the pore or specifically interact with or bind to the nucleotide or polynucleotide thereby facilitating its interaction with the pore.

The molecular adaptor is preferably a cyclic molecule, for example a cyclodextrin, a species that is capable of hybridization, a DNA binder or interchelator, a peptide or peptide analogue, a synthetic polymer, an aromatic planar molecule, a small positively-charged molecule or a small molecule capable of hydrogen-bonding.

The adaptor may be cyclic. A cyclic adaptor preferably has the same symmetry as the pore.

The adaptor typically interacts with the analyte, nucleotide or polynucleotide via host-guest chemistry. The adaptor is typically capable of interacting with the nucleotide or polynucleotide. The adaptor comprises one or more chemical groups that are capable of interacting with the nucleotide or polynucleotide. The one or more chemical groups preferably interact with the nucleotide or polynucleotide by non-covalent interactions, such as hydrophobic interactions, hydrogen bonding, Van der Waal's forces, π-cation interactions and/or electrostatic forces. The one or more chemical groups that are capable of interacting with the nucleotide or polynucleotide are preferably positively charged. The one or more chemical groups that are capable of interacting with the nucleotide or polynucleotide more preferably comprise amino groups. The amino groups can be attached to primary, secondary or tertiary carbon atoms. The adaptor even more preferably comprises a ring of amino groups, such as a ring of 6, 7, 8 or 9 amino groups. The adaptor most preferably comprises a ring of 6 or 9 amino groups. A ring of protonated amino groups may interact with negatively charged phosphate groups in the nucleotide or polynucleotide.

The correct positioning of the adaptor within the pore can be facilitated by host-guest chemistry between the adaptor and the pore comprising the mutant monomer. The adaptor preferably comprises one or more chemical groups that are capable of interacting with one or more amino acids in the pore. The adaptor more preferably comprises one or more chemical groups that are capable of interacting with one or more amino acids in the pore via non-covalent interactions, such as hydrophobic interactions, hydrogen bonding, Van der Waal's forces, π-cation interactions and/or electrostatic forces. The chemical groups that are capable of interacting with one or more amino acids in the pore are typically hydroxyls or amines. The hydroxyl groups can be attached to primary, secondary or tertiary carbon atoms. The hydroxyl groups may form hydrogen bonds with uncharged amino acids in the pore. Any adaptor that facilitates the interaction between the pore and the nucleotide or polynucleotide can be used.

Suitable adaptors include, but are not limited to, cyclodextrins, cyclic peptides and cucurbiturils. The adaptor is preferably a cyclodextrin or a derivative thereof. The cyclodextrin or derivative thereof may be any of those disclosed in Eliseev, A. V., and Schneider, H-J. (1994) J. Am. Chem. Soc. 116, 6081-6088. The adaptor is more preferably heptakis-6-amino-β-cyclodextrin (am7-βCD), 6-monodeoxy-6-monoamino-β-cyclodextrin (am1-βCD) or heptakis-(6-deoxy-6-guanidino)-cyclodextrin (gu7-βCD). The guanidino group in gu7-βCD has a much higher pKa than the primary amines in am7-βCD and so it more positively charged. This gu7-βCD adaptor may be used to increase the dwell time of the nucleotide in the pore, to increase the accuracy of the residual current measured, as well as to increase the base detection rate at high temperatures or low data acquisition rates.

If a succinimidyl 3-(2-pyridyldithio) propionate (SPDP) crosslinker is used as discussed in more detail below, the adaptor is preferably heptakis (6-deoxy-6-amino)-6-N-mono (2-pyridyl)dithiopropanoyl-β-cyclodextrin (am6amPDP1-βCD).

More suitable adaptors include γ-cyclodextrins, which comprise 8 sugar units (and therefore have eight-fold symmetry). The γ-cyclodextrin may contain a linker molecule or may be modified to comprise all or more of the modified sugar units used in the β-cyclodextrin examples discussed above.

The molecular adaptor is preferably covalently attached to the mutant monomer. The adaptor can be covalently attached to the pore using any method known in the art. The adaptor is typically attached via chemical linkage. If the molecular adaptor is attached via cysteine linkage, the one or more cysteines have preferably been introduced to the mutant by substitution. The mutant monomers of the invention can of course comprise a cysteine residue at one or both of positions 272 and 283. The mutant monomer may be chemically modified by attachment of a molecular adaptor to one or both of these cysteines. Alternatively, the mutant monomer may be chemically modified by attachment of a molecule to one or more cysteines or non-natural amino acids, such as FAz, introduced at other positions.

The reactivity of cysteine residues may be enhanced by modification of the adjacent residues. For instance, the basic groups of flanking arginine, histidine or lysine residues will change the pKa of the cysteines thiol group to that of the more reactive S-group. The reactivity of cysteine residues may be protected by thiol protective groups such as dTNB. These may be reacted with one or more cysteine residues of the mutant monomer before a linker is attached.

The molecule may be attached directly to the mutant monomer. The molecule is preferably attached to the mutant monomer using a linker, such as a chemical crosslinker or a peptide linker.

Suitable chemical crosslinkers are well-known in the art. Preferred crosslinkers include 2,5-dioxopyrrolidin-1-yl 3-(pyridin-2-yldisulfanyl) propanoate, 2,5-dioxopyrrolidin-1-yl 4-(pyridin-2-yldisulfanyl) butanoate and 2,5-dioxopyrrolidin-1-yl 8-(pyridin-2-yldisulfanyl) octananoate. The most preferred crosslinker is succinimidyl 3-(2-pyridyldithio) propionate (SPDP). Typically, the molecule is covalently attached to the bifunctional crosslinker before the molecule/crosslinker complex is covalently attached to the mutant monomer but it is also possible to covalently attach the bifunctional crosslinker to the monomer before the bifunctional crosslinker/monomer complex is attached to the molecule.

The linker is preferably resistant to dithiothreitol (DTT). Suitable linkers include, but are not limited to, iodoacetamide-based and Maleimide-based linkers.

In other embodiment, the monomer may be attached to a polynucleotide binding protein. This forms a modular sequencing system that may be used in the methods of the invention. Polynucleotide binding proteins are discussed below.

The polynucleotide binding protein may be covalently attached to the mutant monomer. The protein can be covalently attached to the pore using any method known in the art. The monomer and protein may be chemically fused or genetically fused. The monomer and protein are genetically fused if the whole construct is expressed from a single polynucleotide sequence. Genetic fusion of a pore to a polynucleotide binding protein is discussed in International Application No. PCT/GB09/001679 (published as WO 2010/004265).

The polynucleotide binding protein may be attached directly to the mutant monomer or via one or more linkers. The polynucleotide binding protein may be attached to the mutant monomer using the hybridization linkers described in International Application No. PCT/GB10/000132 (published as WO 2010/086602). Alternatively, peptide linkers may be used. Peptide linkers are amino acid sequences. The length, flexibility and hydrophilicity of the peptide linker are typically designed such that it does not to disturb the functions of the monomer and molecule. Preferred flexible peptide linkers are stretches of 2 to 20, such as 4, 6, 8, 10 or 16, serine and/or glycine amino acids. More preferred flexible linkers include (SG)1, (SG)2, (SG)3, (SG)4, (SG)5 and (SG)8 wherein S is serine and G is glycine. Preferred rigid linkers are stretches of 2 to 30, such as 4, 6, 8, 16 or 24, proline amino acids. More preferred rigid linkers include (P) 12 wherein P is proline.

The mutant monomer may be chemically modified with a molecular adaptor and a polynucleotide binding protein.

### Polynucleotides

The present invention also provides polynucleotide sequences which encode a mutant monomer of the invention. The mutant monomer may be any of those discussed above. The polynucleotide sequence preferably comprises a sequence at least 50%, 60%, 70%, 80%, 90% or 95% homologous based on nucleotide identity to the sequence of SEQ ID NO: 2 over the entire sequence. There may be at least 80%, for example at least 85%, 90% or 95% nucleotide identity over a stretch of 300 or more, for example 375, 450, 525 or 600 or more, contiguous nucleotides (“hard homology”). Homology may be calculated as described above. The polynucleotide sequence may comprise a sequence that differs from SEQ ID NO: 2 on the basis of the degeneracy of the genetic code. The present invention also provides polynucleotide sequences which encode any of the genetically fused constructs of the invention. The polynucleotide preferably comprises two or more variants of the sequence shown in SEQ ID NO: 2. The polynucleotide sequence preferably comprises two or more sequences having at least 50%, 60%, 70%, 80%, 90% or 95% homology to SEQ ID NO: 2 based on nucleotide identity over the entire sequence. There may be at least 80%, for example at least 85%, 90% or 95% nucleotide identity over a stretch of 600 or more, for example 750, 900, 1050 or 1200 or more, contiguous nucleotides (“hard homology”). Homology may be calculated as described above.

Polynucleotide sequences may be derived and replicated using standard methods in the art. Chromosomal DNA encoding wild-type CytK may be extracted from a pore producing organism, such as Bacillus cereus. The gene encoding the pore subunit may be amplified using PCR involving specific primers. The amplified sequence may then undergo site-directed mutagenesis. Suitable methods of site-directed mutagenesis are known in the art and include, for example, combine chain reaction. Polynucleotides encoding a construct of the invention can be made using well-known techniques, such as those described in Sambrook, J. and Russell, D. (2001). Molecular Cloning: A Laboratory Manual, 3rd Edition. Cold Spring Harbor Laboratory Press, Cold Spring Harbor, NY. The resulting polynucleotide sequence may then be incorporated into a recombinant replicable vector such as a cloning vector. The vector may be used to replicate the polynucleotide in a compatible host cell. Thus polynucleotide sequences may be made by introducing a polynucleotide into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The polynucleotide sequence may be cloned into suitable expression vector. In an expression vector, the polynucleotide sequence is typically operably linked to a control sequence which is capable of providing for the expression of the coding sequence by the host cell. Such expression vectors can be used to express a pore subunit.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide sequences may be introduced into the vector.

The expression vector may then be introduced into a suitable host cell. Thus, a mutant monomer or construct of the invention can be produced by inserting a polynucleotide sequence into an expression vector, introducing the vector into a compatible bacterial host cell, and growing the host cell under conditions which bring about expression of the polynucleotide sequence. The recombinantly-expressed monomer or construct may self-assemble into a pore in the host cell membrane. Alternatively, the recombinant pore produced in this manner may be removed from the host cell and inserted into another membrane. When producing pores comprising at least two different monomers or constructs, the different monomers or constructs may be expressed separately in different host cells as described above, removed from the host cells and assembled into a pore in a separate membrane, such as a rabbit cell membrane or a synthetic membrane.

The vectors may be for example, plasmid, virus or phage vectors provided with an origin of replication, optionally a promoter for the expression of the said polynucleotide sequence and optionally a regulator of the promoter. The vectors may contain one or more selectable marker genes, for example a tetracycline resistance gene. Promoters and other expression regulation signals may be selected to be compatible with the host cell for which the expression vector is designed. A T7, trc, lac, ara or λL promoter is typically used. The host cell typically expresses the monomer or construct at a high level. Host cells transformed with a polynucleotide sequence will be chosen to be compatible with the expression vector used to transform the cell. The host cell is typically bacterial and preferably Escherichia coli. Any cell with a λDE3 lysogen, for example C41 (DE3), BL21 (DE3), JM109 (DE3), B834 (DE3), TUNER, Origami and Origami B, can express a vector comprising the T7 promoter.

The invention also comprises a method of producing a mutant monomer of the invention or a construct of the invention. The method comprises expressing a polynucleotide of the invention in a suitable host cell. The polynucleotide is preferably part of a vector and is preferably operably linked to a promoter.

### Making Mutant CytK

The invention also provides a method of improving the ability of a CytK monomer comprising the sequence shown in SEQ ID NO: 1 to characterise a target analyte. The method comprises making one or more modifications between about position S100 and about position K170 of SEQ ID NO: 1 which alter the ability of the monomer to interact with a polynucleotide and do not affect the ability of the monomer to form a pore. Any of the embodiments discussed above with reference to the mutant CytK monomers and below with reference to characterising polynucleotides equally apply to this method of the invention.

### Pores

The invention also provides various pores. The pores of the invention are ideal for characterising analytes. Such pores may be used in the methods provided herein. The pores of the invention are especially ideal for characterising, such as sequencing, polynucleotides because they can discriminate between different nucleotides with a high degree of sensitivity. The pores can be used to characterise nucleic acids, such as DNA and RNA, including sequencing the nucleic acid and identifying single base changes. The pores of the invention can even distinguish between methylated and unmethylated nucleotides. The base resolution of pores of the invention is surprisingly high. The pores show almost complete separation of all four DNA nucleotides. The pores can be further used to discriminate between deoxycytidine monophosphate (dCMP) and methyl-dCMP based on the dwell time in the pore and the current flowing through the pore.

The pores of the invention can also discriminate between different nucleotides under a range of conditions. In particular, the pores will discriminate between nucleotides under conditions that are favourable to the characterising, such as sequencing, of polynucleotides. The extent to which the pores of the invention can discriminate between different nucleotides can be controlled by altering the applied potential, the salt concentration, the buffer, the temperature and the presence of additives, such as urea, betaine and DTT. This allows the function of the pores to be fine-tuned, particularly when sequencing. This is discussed in more detail below. The pores of the invention may also be used to identify polynucleotide polymers from the interaction with one or more monomers rather than on a nucleotide by nucleotide basis.

A pore of the invention may be isolated, substantially isolated, purified or substantially purified. A pore of the invention is isolated or purified if it is completely free of any other components, such as lipids or other pores. A pore is substantially isolated if it is mixed with carriers or diluents which will not interfere with its intended use. For instance, a pore is substantially isolated or substantially purified if it is present in a form that comprises less than 10%, less than 5%, less than 2% or less than 1% of other components, such as lipids or other pores. Alternatively, a pore of the invention may be present in a lipid bilayer.

A pore of the invention may be present as an individual or single pore. Alternatively, a pore of the invention may be present in a homologous or heterologous population or plurality of two or more pores.

### Homo-Oligomeric Pores

The invention also provides a homo-oligomeric pore derived from CytK comprising identical mutant monomers of the invention. The monomers are identical in terms of their amino acid sequence. The homo-oligomeric pore of the invention is ideal for characterising, such as sequencing, polynucleotides. Such pores may be used in the methods provided herein. The homo-oligomeric pore of the invention may have any of the advantages discussed above. The advantages of specific homo-oligomeric pores of the invention are indicated in the Examples.

The homo-oligomeric pore may contain any number of mutant monomers. The pore typically comprises two or more mutant monomers, although typically comprises at least 7, at least 8, at least 9 or at least 10 identical mutant monomers, such as 7, 8, 9 or 10 mutant monomers. Most preferably, the homo-oligomeric pore is a heptameric pore.

One or more of the mutant monomers is preferably chemically modified as discussed above. In other words, one or more of the monomers being chemically modified (and the others not being chemically modified) does not prevent the pore from being homo-oligomeric as long as the amino acid sequence of each of the monomers is identical.

### Hetero-Oligomeric Pores

The invention also provides a hetero-oligomeric pore derived from CytK comprising at least one mutant monomer of the invention, wherein at least one of the monomers differs from the others. The monomer differs from the others in terms of its amino acid sequence. The hetero-oligomeric pore of the invention is ideal for characterising, such as sequencing, polynucleotides. Such pores may be used in the methods provided herein. Hetero-oligomeric pores can be made using methods known in the art (e.g. Protein Sci. 2002 July; 11 (7): 1813-24).

The hetero-oligomeric pore contains sufficient monomers to form the pore. The pore typically comprises two or more mutant monomers, although typically comprises at least 7, at least 8, at least 9 or at least 10 identical mutant monomers, such as 7, 8, 9 or 10 mutant monomers. Most preferably, the hetero-oligomeric pore is a heptameric pore.

In a preferred embodiment, all of the monomers (such as 10, 9, 8 or 7 of the monomers) are mutant monomers of the invention and at least one of them differs from the others. In a more preferred embodiment, the pore comprises eight or nine mutant monomers of the invention and at least one of them differs from the others. They may all differ from one another.

The mutant monomers of the invention in the pore are preferably approximately the same length or are the same length. The barrels of the mutant monomers of the invention in the pore are preferably approximately the same length or are the same length. Length may be measured in number of amino acids and/or units of length.

In another preferred embodiment, at least one of the mutant monomers is not a mutant monomer of the invention. In this embodiment, the remaining monomers are preferably mutant monomers of the invention. Hence, the pore may comprise 9, 8, 7, 6, 5, 4, 3, 2 or 1 mutant monomers of the invention. Any number of the monomers in the pore may not be a mutant monomer of the invention. The pore preferably comprises seven or eight mutant monomers of the invention and a monomer which is not a monomer of the invention. The mutant monomers of the invention may be the same or different.

The mutant monomers of the invention in the construct are preferably approximately the same length or are the same length. The barrels of the mutant monomers of the invention in the construct are preferably approximately the same length or are the same length. Length may be measured in number of amino acids and/or units of length.

The pore may comprise one or more monomers which are not mutant monomers of the invention.

Methods for making pores are discussed in more detail below.

### Construct

The invention also provides a construct comprising two or more covalently attached monomers derived from CytK, wherein at least one of the monomers is a mutant monomer of the invention. The construct of the invention retains its ability to form a pore. This may be determined as discussed above. One or more constructs of the invention may be used to form pores for characterising, such as sequencing, polypeptides or polynucleotides. Such pores may be used in the methods provided herein. The construct may comprise at least 2, at least 3, at least 4, at least 5, at least 6, at least 7, at least 8, at least 9 or at least 10 monomers. The construct preferably comprises two monomers. The two or more monomers may be the same or different.

At least one monomer in the construct is a mutant monomer of the invention. 2 or more, 3 or more, 4 or more, 5 or more, 6 or more, 7 or more, 8 or more, 9 or more or 10 or more monomers in the construct may be mutant monomers of the invention. All of the monomers in the construct are preferably mutant monomers of the invention. The mutant monomers may be the same or different. In a preferred embodiment, the construct comprises two mutant monomers of the invention.

The monomers in the construct are preferably genetically fused. Monomers are genetically fused if the whole construct is expressed from a single polynucleotide sequence. The coding sequences of the monomers may be combined in any way to form a single polynucleotide sequence encoding the construct.

The monomers may be genetically fused in any configuration. The monomers may be fused via their terminal amino acids. For instance, the amino terminus of the one monomer may be fused to the carboxy terminus of another monomer. The second and subsequent monomers in the construct (in the amino to carboxy direction) may comprise a methionine at their amino terminal ends (each of which is fused to the carboxy terminus of the previous monomer). For instance, if M is a monomer (without an amino terminal methionine) and mM is a monomer with an amino terminal methionine, the construct may comprise the sequence M-mM, M-mM-mM or M-mM-mM-mM. The presences of these methionines typically results from the expression of the start codons (i.e. ATGs) at the 5′ end of the polynucleotides encoding the second or subsequent monomers within the polynucleotide encoding entire construct. The first monomer in the construct (in the amino to carboxy direction) may also comprise a methionine (e.g. mM-mM, mM-mM-mM or mM-mM-mM-mM).

The two or more monomers may be fused directly together. The monomers are preferably fused using a linker. The linker may be designed to constrain the mobility of the monomers. Preferred linkers are amino acid sequences (i.e. peptide linkers). Any of the peptide linkers discussed above may be used.

In another preferred embodiment, the monomers are chemically fused. Two monomers are chemically fused if the two parts are chemically attached, for instance via a chemical crosslinker. Any of the chemical crosslinkers discussed above may be used. The linker may be attached to one or more cysteine residues introduced into a mutant monomer of the invention. Alternatively, the linker may be attached to a terminus of one of the monomers in the construct.

If a construct contains different monomers, crosslinkage of monomers to themselves may be prevented by keeping the concentration of linker in a vast excess of the monomers. Alternatively, a “lock and key” arrangement may be used in which two linkers are used. Only one end of each linker may react together to form a longer linker and the other ends of the linker each react with a different monomers. Such linkers are described in International Application No. PCT/GB 10/000132 (published as WO 2010/086602).

### Construct-Containing Pores

The invention also provides a pore comprising at least one construct of the invention. Such pores may be used in the methods provided herein. A construct of the invention comprises two or more covalently attached monomers derived from CytK, wherein at least one of the monomers is a mutant CytK monomer of the invention. In other words, a construct must contain more than one monomer. At least two of the monomers in the pore are in the form of a construct of the invention. The monomers may be of any type.

A pore typically contains (a) one construct comprising two monomers and (b) a sufficient number of monomers to form the pore. The construct may be any of those discussed above. The monomers may be any of those discussed above, including mutant monomers of the invention.

Another typical pore comprises more than one construct of the invention, such as two, three or four constructs of the invention. Such pores further comprise a sufficient number of monomers to form the pore. The monomer may be any of those discussed above. A further pore of the invention comprises only constructs comprising 2 monomers. A specific pore according to the invention comprises several constructs each comprising two monomers. The constructs may oligomerise into a pore with a structure such that only one monomer from each construct contributes to the pore. Typically, the other monomers of the construct (i.e. the ones not forming the pore) will be on the outside of the pore. Mutations can be introduced into the construct as described above. The mutations may be alternating, i.e. the mutations are different for each monomer within a two monomer construct and the constructs are assembled as a homo-oligomer resulting in alternating modifications. In other words, monomers comprising MutA and MutB are fused and assembled to form an A-B: A-B: A-B: A-B pore. Alternatively, the mutations may be neighbouring, i.e. identical mutations are introduced into two monomers in a construct and this is then oligomerised with different mutant monomers. In other words, monomers comprising MutA are fused follow by oligomerisation with MutB-containing monomers to form A-A: B: B: B: B: B: B.

One or more of the monomers of the invention in a construct-containing pore may be chemically-modified as discussed above.

### Producing Pores of the Invention

The invention also provides a method of producing a pore of the invention. The method comprises allowing at least one mutant monomer of the invention or at least one construct of the invention to oligomerise with a sufficient number of mutant CytK monomers of the invention, constructs of the invention or monomers derived from CytK to form a pore. If the method concerns making a homo-oligomeric pore of the invention, all of the monomers used in the method are mutant CytK monomers of the invention having the same amino acid sequence. If the method concerns making a hetero-oligomeric pore of the invention, at least one of the monomers is different from the others. Any of the embodiments discussed above with reference to the pores of the invention equally apply to the methods of producing the pores.

A preferred way of making a pore of the invention is disclosed in Example 1.

### Membrane

The pore of the invention may be present in a membrane. Accordingly, the invention provides a membrane comprising a pore of the invention.

In the methods of the invention, the polynucleotide is typically contacted with the pore of the invention in a membrane. Any membrane may be used in accordance with the invention. Suitable membranes are well-known in the art. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphipiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer based membranes for a wide range of applications.

The membrane is most preferably one of the membranes disclosed in International Application No. PCT/GB2013/052766 or PCT/GB2013/052767.

The amphiphilic molecules may be chemically-modified or functionalised to facilitate coupling of the polynucleotide.

The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10-8 cm s-1. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in International Application No. PCT/GB08/000563 (published as WO 2008/102121), International Application No. PCT/GB08/004127 (published as WO 2009/077734) and International Application No. PCT/GB2006/001057 (published as WO 2006/100484).

Methods for forming lipid bilayers are known in the art. Lipid bilayers are commonly formed by the method of Montal and Mueller (Proc. Natl. Acad. Sci. USA., 1972; 69:3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

In a preferred embodiment, the lipid bilayer is formed as described in International Application No. PCT/GB08/004127 (published as WO 2009/077734). Advantageously in this method, the lipid bilayer is formed from dried lipids. In a most preferred embodiment, the lipid bilayer is formed across an opening as described in WO2009/077734 (PCT/GB08/004127).

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalised PEG Lipids, such as 1,2-Distearoyl-sn-Glycero-3 Phosphoethanolamine-N-[Biotinyl(Polyethylene Glycol) 2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

In another preferred embodiment, the membrane comprises a solid state layer. Solid state layers can be formed from both organic and inorganic materials including, but not limited to, microelectronic materials, insulating materials such as Si3N4, Al2O3, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in International Application No. PCT/US2008/010637 (published as WO 2009/035647). If the membrane comprises a solid state layer, the pore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The method of the invention described herein is typically carried out using (i) an artificial amphiphilic layer comprising a pore, (ii) an isolated, naturally-occurring lipid bilayer comprising a pore, or (iii) a cell having a pore inserted therein. The method is typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

### Array

The invention also provides an array comprising a plurality of membranes of the invention. In a preferred embodiment, each membrane in the array comprises one pore of the invention.

The array is preferably set up to carry out the method of characterising analytes described herein. For example, the array may form part of an apparatus comprising a chamber further comprising an aqueous solution and a barrier that separates the chamber into two sections. The barrier typically has an aperture in which the membrane containing the pore is formed. Alternatively the barrier forms the membrane in which the pore is present.

### Device

The invention also provides a device comprising the array of the invention, means for applying a potential across the membranes, and means for detecting electrical or optical signals across the membrane. The device of the invention is preferably set up to carry out the method of characterising analytes described herein.

Preferably, the device comprises an electrical circuit capable of applying a potential and measuring an electrical signal across the membrane and pore.

The device preferably is capable of supporting the plurality of pores and membranes and being operable to perform analyte characterisation using the pores and membrane in accordance with the method of characterising analytes described herein. The device particularly may comprise at least one reservoir for holding material for performing the characterising; a fluidics system configured to controllably supply material from the at least one reservoir to the sensor device; and one or more containers for receiving respective samples, the fluidics system being configured to supply the samples selectively from one or more containers to the device

### Method of Characterising Analytes

The invention provides a method of determining the presence, absence or one or more characteristics of a target analyte. In particular, the method is for characterising a target analyte. The method of characterising a target analyte comprises:
(a) contacting the target analyte with a pore according to the invention such that the target analyte moves with respect to the pore; and
(b) taking one or more measurements characteristic of the analyte as the analyte moves with respect to the pore,
thereby characterising the target analyte.

Steps (a) and (b) of the method are preferably carried out with a potential applied across the pore. As discussed in more detail below, the applied potential typically results in the formation of a complex between the pore and a polynucleotide binding protein. The applied potential may be a voltage potential. Alternatively, the applied potential may be a chemical potential. An example of this is using a salt gradient across an amphiphilic layer. A salt gradient is disclosed in Holden et al., J Am Chem Soc. 2007 Jul. 11; 129 (27): 8650-5.

The method is for determining the presence, absence or one or more characteristics of a target analyte. The method may be for determining the presence, absence or one or more characteristics of at least one analyte. The method may concern determining the presence, absence or one or more characteristics of two or more analytes. The method may comprise determining the presence, absence or one or more characteristics of any number of analytes, such as 2, 5, 10, 15, 20, 30, 40, 50, 100 or more analytes. Any number of characteristics of the one or more analytes may be determined, such as 1, 2, 3, 4, 5, 10 or more characteristics.

The target analyte is preferably a metal ion, an inorganic salt, a polymer, an amino acid, a peptide, a polypeptide, a protein, a nucleotide, an oligonucleotide, a polynucleotide, an oligosaccharide. The method may concern determining the presence, absence or one or more characteristics of two or more analytes of the same type, such as two or more proteins, two or more nucleotides or two or more pharmaceuticals. Alternatively, the method may concern determining the presence, absence or one or more characteristics of two or more analytes of different types, such as one or more proteins, one or more nucleotides and one or more pharmaceuticals.

The target analyte can be secreted from cells. Alternatively, the target analyte can be an analyte that is present inside cells such that the analyte must be extracted from the cells before the invention can be carried out.

The analyte is preferably an amino acid, a peptide, a polypeptides and/or a protein. The amino acid, peptide, polypeptide or protein can be naturally-occurring or non-naturally-occurring. The polypeptide or protein can include within them synthetic or modified amino acids. A number of different types of modification to amino acids are known in the art. Suitable amino acids and modifications thereof are above. For the purposes of the invention, it is to be understood that the target analyte can be modified by any method available in the art.

The protein can be an enzyme, an antibody, a hormone, a growth factor or a growth regulatory protein, such as a cytokine. The cytokine may be selected from interleukins, preferably IFN-1, IL-1, IL-2, IL-4, IL-5, IL-6, IL-10, IL-12 and IL-13, interferons, preferably IL-g, and other cytokines such as TNF-α. The protein may be a bacterial protein, a fungal protein, a virus protein or a parasite-derived protein.

The target analyte is preferably a nucleotide, an oligonucleotide or a polynucleotide. Nucleotides and polynucleotides are discussed below. Oligonucleotides are short nucleotide polymers which typically have 50 or fewer nucleotides, such 40 or fewer, 30 or fewer, 20 or fewer, 10 or fewer or 5 or fewer nucleotides. The oligonucleotides may comprise any of the nucleotides discussed below, including the abasic and modified nucleotides.

The target analyte, such as a target polynucleotide, may be present in any of the suitable samples discussed below.

The pore is typically present in a membrane as discussed below. The target analyte may be coupled or delivered to the membrane using of the methods discussed below.

Any of the measurements discussed below can be used to determine the presence, absence or one or more characteristics of the target analyte. The method preferably comprises contacting the target analyte with the pore such that the analyte moves with respect to, such as moves through, the pore and measuring the current passing through the pore as the analyte moves with respect to the pore and thereby determining the presence, absence or one or more characteristics of the analyte.

The target analyte is present if the current flows through the pore in a manner specific for the analyte (i.e. if a distinctive current associated with the analyte is detected flowing through the pore). The analyte is absent if the current does not flow through the pore in a manner specific for the nucleotide. Control experiments can be carried out in the presence of the analyte to determine the way in which if affects the current flowing through the pore.

The invention can be used to differentiate analytes of similar structure on the basis of the different effects they have on the current passing through a pore. Individual analytes can be identified at the single molecule level from their current amplitude when they interact with the pore. The invention can also be used to determine whether or not a particular analyte is present in a sample. The invention can also be used to measure the concentration of a particular analyte in a sample. Analyte characterisation using pores other than CytK is known in the art.

### Polynucleotide Characterisation

The methods of the invention may be utilised to characterise target polynucleotides. The invention may therefore provide a method of characterising a target polynucleotide, such as sequencing a polynucleotide. There are two main strategies for characterising or sequencing polynucleotides using nanopores, namely strand characterisation/sequencing and exonuclease characterisation/sequencing. The method of the invention may concern either method.

In strand sequencing, the DNA is translocated through the nanopore either with or against an applied potential. Exonucleases that act progressively or processively on double stranded DNA can be used on the cis side of the pore to feed the remaining single strand through under an applied potential or the trans side under a reverse potential. Likewise, a helicase that unwinds the double stranded DNA can also be used in a similar manner. A polymerase may also be used. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential.

In one embodiment, the method of characterising a target polynucleotide involves contacting the target sequence with a pore of the invention and a helicase enzyme. Any helicase may be used in the method. Suitable helicases are discussed below. Helicases may work in two modes with respect to the pore. First, the method is preferably carried out using a helicase such that it controls movement of the target sequence through the pore with the field resulting from the applied voltage. In this mode the 5′ end of the DNA is first captured in the pore, and the enzyme controls movement of the DNA into the pore such that the target sequence is passed through the pore with the field until it finally translocates through to the trans side of the bilayer. Alternatively, the method is preferably carried out such that a helicase enzyme controls movement of the target sequence through the pore against the field resulting from the applied voltage. In this mode the 3′ end of the DNA is first captured in the pore, and the enzyme controls movement of the DNA through the pore such that the target sequence is pulled out of the pore against the applied field until finally ejected back to the cis side of the bilayer.

In exonuclease sequencing, an exonuclease releases individual nucleotides from one end of the target polynucleotide and these individual nucleotides are identified as discussed below. In another embodiment, the method of characterising a target polynucleotide involves contacting the target sequence with a pore and an exonuclease enzyme. Any of the exonuclease enzymes discussed below may be used in the method. The enzyme may be covalently attached to the pore as discussed below.

Exonucleases are enzymes that typically latch onto one end of a polynucleotide and digest the sequence one nucleotide at a time from that end. The exonuclease can digest the polynucleotide in the 5′ to 3′ direction or 3′ to 5′ direction. The end of the polynucleotide to which the exonuclease binds is typically determined through the choice of enzyme used and/or using methods known in the art. Hydroxyl groups or cap structures at either end of the polynucleotide may typically be used to prevent or facilitate the binding of the exonuclease to a particular end of the polynucleotide.

The method involves contacting the polynucleotide with the exonuclease so that the nucleotides are digested from the end of the polynucleotide at a rate that allows characterisation or identification of a proportion of nucleotides as discussed above. Methods for doing this are well known in the art. For example, Edman degradation is used to successively digest single amino acids from the end of polypeptide such that they may be identified using High Performance Liquid Chromatography (HPLC). A homologous method may be used in the present invention.

The rate at which the exonuclease functions is typically slower than the optimal rate of a wild-type exonuclease. A suitable rate of activity of the exonuclease in the method of the invention involves digestion of from 0.5 to 1000 nucleotides per second, from 0.6 to 500 nucleotides per second, 0.7 to 200 nucleotides per second, from 0.8 to 100 nucleotides per second, from 0.9 to 50 nucleotides per second or 1 to 20 or 10 nucleotides per second. The rate is preferably 1, 10, 100, 500 or 1000 nucleotides per second. A suitable rate of exonuclease activity can be achieved in various ways. For example, variant exonucleases with a reduced optimal rate of activity may be used in accordance with the invention.

In the strand characterisation embodiment, the method comprises contacting the polynucleotide with a pore of the invention such that the polynucleotide moves with respect to, such as through, the pore and taking one or more measurements as the polynucleotide moves with respect to the pore, wherein the measurements are indicative of one or more characteristics of the polynucleotide, and thereby characterising the target polynucleotide.

In the exonucleotide characterisation embodiment, the method comprises contacting the polynucleotide with a pore of the invention and an exonucleoase such that the exonuclease digests individual nucleotides from one end of the target polynucleotide and the individual nucleotides move with respect to, such as through, the pore and taking one or more measurements as the individual nucleotides move with respect to the pore, wherein the measurements are indicative of one or more characteristics of the individual nucleotides, and thereby characterising the target polynucleotide.

An individual nucleotide is a single nucleotide. An individual nucleotide is one which is not bound to another nucleotide or polynucleotide by a nucleotide bond. A nucleotide bond involves one of the phosphate groups of a nucleotide being bound to the sugar group of another nucleotide. An individual nucleotide is typically one which is not bound by a nucleotide bond to another polynucleotide of at least 5, at least 10, at least 20, at least 50, at least 100, at least 200, at least 500, at least 1000 or at least 5000 nucleotides. For example, the individual nucleotide has been digested from a target polynucleotide sequence, such as a DNA or RNA strand. The nucleotide can be any of those discussed below.

The individual nucleotides may interact with the pore in any manner and at any site. The nucleotides preferably reversibly bind to the pore via or in conjunction with an adaptor as discussed above. The nucleotides most preferably reversibly bind to the pore via or in conjunction with the adaptor as they pass through the pore across the membrane. The nucleotides can also reversibly bind to the barrel or channel of the pore via or in conjunction with the adaptor as they pass through the pore across the membrane.

During the interaction between the individual nucleotide and the pore, the nucleotide typically affects the current flowing through the pore in a manner specific for that nucleotide. For example, a particular nucleotide will reduce the current flowing through the pore for a particular mean time period and to a particular extent. In other words, the current flowing through the pore is distinctive for a particular nucleotide. Control experiments may be carried out to determine the effect a particular nucleotide has on the current flowing through the pore. Results from carrying out the method of the invention on a test sample can then be compared with those derived from such a control experiment in order to identify a particular nucleotide in the sample or determine whether a particular nucleotide is present in the sample. The frequency at which the current flowing through the pore is affected in a manner indicative of a particular nucleotide can be used to determine the concentration of that nucleotide in the sample. The ratio of different nucleotides within a sample can also be calculated. For instance, the ratio of dCMP to methyl-dCMP can be calculated.

The method involves measuring one or more characteristics of the target polynucleotide. The target polynucleotide may also be called the template polynucleotide or the polynucleotide of interest.

This embodiment also uses a pore of the invention. Any of the pores and embodiments discussed above with reference to the target analyte may be used.

### Polynucleotide Analyte

A polynucleotide, such as a nucleic acid, is a macromolecule comprising two or more nucleotides. The polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag. Suitable labels are described below. The polynucleotide may comprise one or more spacers.

A nucleotide typically contains a nucleobase, a sugar and at least one phosphate group. The nucleobase and sugar form a nucleoside.

The nucleobase is typically heterocyclic. Nucleobases include, but are not limited to, purines and pyrimidines and more specifically adenine (A), guanine (G), thymine (T), uracil (U) and cytosine (C).

The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC). The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. Nucleotides include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP.

A nucleotide may be abasic (i.e. lack a nucleobase). A nucleotide may also lack a nucleobase and a sugar (i.e. is a C3 spacer).

The nucleotides in the polynucleotide may be attached to each other in any manner. The nucleotides are typically attached by their sugar and phosphate groups as in nucleic acids. The nucleotides may be connected via their nucleobases as in pyrimidine dimers. The polynucleotide may be single stranded or double stranded. The polynucleotide is preferably single stranded. Single stranded polynucleotide characterization is referred to as 1D in the Examples. At least a portion of the polynucleotide may be double stranded. The polynucleotide can be a nucleic acid, such as deoxyribonucleic acid (DNA) or ribonucleic acid (RNA). The polynucleotide can comprise one strand of RNA hybridised to one strand of DNA. The polynucleotide may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The PNA backbone is composed of repeating N-(2-aminoethyl)-glycine units linked by peptide bonds. The GNA backbone is composed of repeating glycol units linked by phosphodiester bonds. The TNA backbone is composed of repeating threose sugars linked together by phosphodiester bonds. LNA is formed from ribonucleotides as discussed above having an extra bridge connecting the 2′ oxygen and 4′ carbon in the ribose moiety. Bridged nucleic acids (BNAs) are modified RNA nucleotides. They may also be called constrained or inaccessible RNA. BNA monomers can contain a five-membered, six-membered or even a seven-membered bridged structure with a “fixed” C3′-endo sugar puckering. The bridge is synthetically incorporated at the 2′,4′-position of the ribose to produce a 2′,4′-BNA monomer.

The polynucleotide is most preferably ribonucleic nucleic acid (RNA) or deoxyribonucleic acid (DNA).

The polynucleotide can be any length. For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length.

Any number of polynucleotides can be investigated. For instance, the method of the invention may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide.

The polynucleotide can be naturally occurring or artificial. For instance, the method may be used to verify the sequence of a manufactured oligonucleotide. The method is typically carried out in vitro.

### Sample

The polynucleotide is typically present in any suitable sample. The invention is typically carried out on a sample that is known to contain or suspected to contain the polynucleotide. Alternatively, the invention may be carried out on a sample to confirm the identity of a polynucleotide whose presence in the sample is known or expected.

The sample may be a biological sample. The invention may be carried out in vitro using a sample obtained from or extracted from any organism or microorganism. The organism or microorganism is typically archaeal, prokaryotic or eukaryotic and typically belongs to one of the five kingdoms: plantae, animalia, fungi, monera and protista. The invention may be carried out in vitro on a sample obtained from or extracted from any virus. The sample is preferably a fluid sample. The sample typically comprises a body fluid of the patient. The sample may be urine, lymph, saliva, mucus or amniotic fluid but is preferably blood, plasma or serum.

Typically, the sample is human in origin, but alternatively it may be from another mammal animal such as from commercially farmed animals such as horses, cattle, sheep, fish, chickens or pigs or may alternatively be pets such as cats or dogs. Alternatively, the sample may be of plant origin, such as a sample obtained from a commercial crop, such as a cereal, legume, fruit or vegetable, for example wheat, barley, oats, canola, maize, soya, rice, rhubarb, bananas, apples, tomatoes, potatoes, grapes, tobacco, beans, lentils, sugar cane, cocoa, cotton.

The sample may be a non-biological sample. The non-biological sample is preferably a fluid sample. Examples of non-biological samples include surgical fluids, water such as drinking water, sea water or river water, and reagents for laboratory tests. The sample is typically processed prior to being used in the invention, for example by centrifugation or by passage through a membrane that filters out unwanted molecules or cells, such as red blood cells. The may be measured immediately upon being taken. The sample may also be typically stored prior to assay, preferably below −70° C.

### Polynucleotide Characterisation

The method may involve measuring two, three, four or five or more characteristics of the polynucleotide. The one or more characteristics are preferably selected from (i) the length of the polynucleotide, (ii) the identity of the polynucleotide, (iii) the sequence of the polynucleotide, (iv) the secondary structure of the polynucleotide and (v) whether or not the polynucleotide is modified. Any combination of (i) to (v) may be measured in accordance with the invention, such as {i}, {ii}, {iii}, {iv}, {v}, {i,ii}, {i,iii}, {i,iv}, {i,v}, {ii,iii}, {ii,iv}, {ii,v}, {iii,iv}, {iii,v}, {iv,v}, {i,ii,iii}, {i,ii,iv}, {i,ii,v}, {i,iii,iv}, {i,iii,v}, {i,iv,v}, {ii,iii,iv}, {ii,iii,v}, {ii,iv,v}, {iii,iv,v}, {i,ii,iii,iv}, {i,ii,iii,v}, {i,ii,iv,v}, {i,iii,iv,v}, {ii,iii,iv,v} or {i,ii,iii,iv,v}. Different combinations of (i) to (v) may be measured for the first polynucleotide compared with the second polynucleotide, including any of those combinations listed above.

For (i), the length of the polynucleotide may be measured for example by determining the number of interactions between the polynucleotide and the pore or the duration of interaction between the polynucleotide and the pore.

For (ii), the identity of the polynucleotide may be measured in a number of ways. The identity of the polynucleotide may be measured in conjunction with measurement of the sequence of the polynucleotide or without measurement of the sequence of the polynucleotide. The former is straightforward; the polynucleotide is sequenced and thereby identified. The latter may be done in several ways. For instance, the presence of a particular motif in the polynucleotide may be measured (without measuring the remaining sequence of the polynucleotide). Alternatively, the measurement of a particular electrical and/or optical signal in the method may identify the polynucleotide as coming from a particular source.

For (iii), the sequence of the polynucleotide can be determined as described previously. Suitable sequencing methods, particularly those using electrical measurements, are described in Stoddart D et al., Proc Natl Acad Sci, 12; 106 (19): 7702-7, Lieberman K R et al, J Am Chem Soc. 2010; 132 (50): 17961-72, and International Application WO 2000/28312.

For (iv), the secondary structure may be measured in a variety of ways. For instance, if the method involves an electrical measurement, the secondary structure may be measured using a change in dwell time or a change in current flowing through the pore. This allows regions of single-stranded and double-stranded polynucleotide to be distinguished.

For (v), the presence or absence of any modification may be measured. The method preferably comprises determining whether or not the polynucleotide is modified by methylation, by oxidation, by damage, with one or more proteins or with one or more labels, tags or spacers. Specific modifications will result in specific interactions with the pore which can be measured using the methods described below. For instance, methylcyotsine may be distinguished from cytosine on the basis of the current flowing through the pore during its interaction with each nucleotide.

The target polynucleotide is contacted with a pore of the invention. The pore is typically present in a membrane. Suitable membranes are discussed below. The method may be carried out using any apparatus that is suitable for investigating a membrane/pore system in which a pore is present in a membrane. The method may be carried out using any apparatus that is suitable for transmembrane pore sensing. For example, the apparatus comprises a chamber comprising an aqueous solution and a barrier that separates the chamber into two sections. The barrier typically has an aperture in which the membrane containing the pore is formed. Alternatively the barrier forms the membrane in which the pore is present.

The method may be carried out using the apparatus described in International Application No. PCT/GB08/000562 (WO 2008/102120).

A variety of different types of measurements may be made. This includes without limitation: electrical measurements and optical measurements. Possible electrical measurements include: current measurements, impedance measurements, tunnelling measurements (Ivanov A P et al., Nano Lett. 2011 Jan. 12; 11 (1): 279-85), and FET measurements (International Application WO 2005/124888). Optical measurements may be combined with electrical measurements (Soni G V et al., Rev Sci Instrum. 2010 January; 81 (1): 014301; Chen C., et al “High spatial resolution nanoslit SERS for single-molecule nucleobase sensing.” Nat. Comm. (2018) 9:1733). The measurement may be a transmembrane current measurement such as measurement of ionic current flowing through the pore.

Electrical measurements may be made using standard single channel recording equipment as describe in Stoddart D et al., Proc Natl Acad Sci, 12; 106 (19): 7702-7, Lieberman K R et al, J Am Chem Soc. 2010; 132 (50): 17961-72, and International Application WO 2000/28312. Alternatively, electrical measurements may be made using a multi-channel system, for example as described in International Application WO 2009/077734 and International Application WO 2011/067559.

The method is preferably carried out with a potential applied across the membrane. The applied potential may be a voltage potential. Alternatively, the applied potential may be a chemical potential. An example of this is using a salt gradient across a membrane, such as an amphiphilic layer. A salt gradient is disclosed in Holden et al., J Am Chem Soc. 2007 Jul. 11; 129 (27): 8650-5. In some instances, the current passing through the pore as a polynucleotide moves with respect to the pore is used to estimate or determine the sequence of the polynucleotide. This is strand sequencing.

The method may involve measuring the current passing through the pore as the polynucleotide moves with respect to the pore. Therefore the apparatus used in the method may also comprise an electrical circuit capable of applying a potential and measuring an electrical signal across the membrane and pore. The methods may be carried out using a patch clamp or a voltage clamp. The methods preferably involve the use of a voltage clamp.

The method of the invention may involve the measuring of a current passing through the pore as the polynucleotide moves with respect to the pore. Suitable conditions for measuring ionic currents through transmembrane protein pores are known in the art and disclosed in the Example. The method is typically carried out with a voltage applied across the membrane and pore. The voltage used is typically from +5 V to −5 V, such as from +4 V to −4 V, +3 V to −3 V or +2 V to −2 V. The voltage used is typically from −600 mV to +600 mV or −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The method is typically carried out in the presence of any charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane. The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations. The method is typically carried out in the presence of a buffer. In the exemplary apparatus discussed above, the buffer is present in the aqueous solution in the chamber. Any buffer may be used in the method of the invention. Typically, the buffer is phosphate buffer. Other suitable buffers are HEPES and Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The method may be carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods are typically carried out at room temperature. The methods are optionally carried out at a temperature that supports enzyme function, such as about 37° C.

### Polynucleotide Binding Protein

The strand characterisation method preferably comprises contacting the polynucleotide with a polynucleotide binding protein such that the protein controls the movement of the polynucleotide with respect to, such as through, the pore.

More preferably, the method comprises (a) contacting the polynucleotide with a a pore of the invention and a polynucleotide binding protein such that the protein controls the movement of the polynucleotide with respect to, such as through, the pore and (b) taking one or more measurements as the polynucleotide moves with respect to the pore, wherein the measurements are indicative of one or more characteristics of the polynucleotide, and thereby characterising the polynucleotide.

More preferably, the method comprises (a) contacting the polynucleotide with a a pore of the invention and a polynucleotide binding protein such that the protein controls the movement of the polynucleotide with respect to, such as through, the pore and (b) measuring the current through the pore as the polynucleotide moves with respect to the pore, wherein the current is indicative of one or more characteristics of the polynucleotide, and thereby characterising the polynucleotide.

The polynucleotide binding protein may be any protein that is capable of binding to the polynucleotide and controlling its movement through the pore. It is straightforward in the art to determine whether or not a protein binds to a polynucleotide. The protein typically interacts with and modifies at least one property of the polynucleotide. The protein may modify the polynucleotide by cleaving it to form individual nucleotides or shorter chains of nucleotides, such as di- or trinucleotides. The protein may modify the polynucleotide by orienting it or moving it to a specific position, i.e. controlling its movement.

The polynucleotide binding protein is preferably derived from a polynucleotide handling enzyme. A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The polynucleotide handling enzyme does not need to display enzymatic activity as long as it is capable of binding the polynucleotide and controlling its movement through the pore. For instance, the enzyme may be modified to remove its enzymatic activity or may be used under conditions which prevent it from acting as an enzyme. Such conditions are discussed in more detail below.

The polynucleotide handling enzyme is preferably derived from a nucleolytic enzyme. The polynucleotide handling enzyme used in the construct of the enzyme is more preferably derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31. The enzyme may be any of those disclosed in International Application No. PCT/GB10/000133 (published as WO 2010/086603).

Preferred enzymes are polymerases, exonucleases, helicases and topoisomerases, such as gyrases. Suitable enzymes include, but are not limited to, exonuclease I from E. coli (SEQ ID NO: 3), exonuclease III enzyme from E. coli (SEQ ID NO: 4), RecJ from T. thermophilus (SEQ ID NO: 5) and bacteriophage lambda exonuclease (SEQ ID NO: 6), TatD exonuclease and variants thereof. Three subunits comprising the sequence shown in SEQ ID NO: 5 or a variant thereof interact to form a trimer exonuclease. These exonucleases can also be used in the exonuclease method of the invention. The polymerase may be PyroPhage® 3173 DNA Polymerase (which is commercially available from Lucigen® Corporation), SD Polymerase (commercially available from Bioron®) or variants thereof. The enzyme is preferably Phi29 DNA polymerase (SEQ ID NO: 7) or a variant thereof. The topoisomerase is preferably a member of any of the Moiety Classification (EC) groups 5.99.1.2 and 5.99.1.3.

The enzyme is most preferably derived from a helicase, such as Hel308 Mbu (SEQ ID NO: 8), Hel308 Csy (SEQ ID NO: 9), Hel308 Tga (SEQ ID NO: 10), Hel308 Mhu (SEQ ID NO: 11), TraI Eco (SEQ ID NO: 12), XPD Mbu (SEQ ID NO: 13) or a variant thereof. Any helicase may be used in the invention. The helicase may be or be derived from a Hel308 helicase, a RecD helicase, such as TraI helicase or a TrwC helicase, a XPD helicase or a Dda helicase. The helicase may be any of the helicases, modified helicases or helicase constructs disclosed in International Application Nos. PCT/GB2012/052579 (published as WO 2013/057495); PCT/GB2012/053274 (published as WO 2013/098562); PCT/GB2012/053273 (published as WO2013098561); PCT/GB2013/051925 (published as WO 2014/013260); PCT/GB2013/051924 (published as WO 2014/013259); PCT/GB2013/051928 (published as WO 2014/013262) and PCT/GB2014/052736.

The helicase preferably comprises the sequence shown in SEQ ID NO: 15 (Trwc Cba) or as variant thereof, the sequence shown in SEQ ID NO: 8 (Hel308 Mbu) or a variant thereof or the sequence shown in SEQ ID NO: 14 (Dda) or a variant thereof. Variants may differ from the native sequences in any of the ways discussed below for transmembrane pores. A preferred variant of SEQ ID NO: 14 comprises (a) E94C and A360C or (b) E94C, A360C, C109A and C136A and then optionally (AM1) G1G2 (i.e. deletion of M1 and then addition G1 and G2).

Any number of helicases may be used in accordance with the invention. For instance, 1, 2, 3, 4, 5, 6, 7, 8, 9, 10 or more helicases may be used. In some embodiments, different numbers of helicases may be used.

The method of the invention preferably comprises contacting the polynucleotide with two or more helicases. The two or more helicases are typically the same helicase. The two or more helicases may be different helicases.

The two or more helicases may be any combination of the helicases mentioned above. The two or more helicases may be two or more Dda helicases. The two or more helicases may be one or more Dda helicases and one or more TrwC helicases. The two or more helicases may be different variants of the same helicase.

The two or more helicases are preferably attached to one another. The two or more helicases are more preferably covalently attached to one another. The helicases may be attached in any order and using any method. Preferred helicase constructs for use in the invention are described in International Application Nos. PCT/GB2013/051925 (published as WO 2014/013260); PCT/GB2013/051924 (published as WO 2014/013259); PCT/GB2013/051928 (published as WO 2014/013262) and PCT/GB2014/052736.

A variant of SEQ ID NOs: 7, 3, 4, 5, 16, 8, 9, 10, 11, 12, 13, 14 or 15 is an enzyme that has an amino acid sequence which varies from that of SEQ ID NO: 7, 3, 4, 5, 16, 8, 9, 10, 11, 12, 13, 14 or 15 and which retains polynucleotide binding ability. This can be measured using any method known in the art. For instance, the variant can be contacted with a polynucleotide and its ability to bind to and move along the polynucleotide can be measured. The variant may include modifications that facilitate binding of the polynucleotide and/or facilitate its activity at high salt concentrations and/or room temperature. Variants may be modified such that they bind polynucleotides (i.e. retain polynucleotide binding ability) but do not function as a helicase (i.e. do not move along polynucleotides when provided with all the necessary components to facilitate movement, e.g. ATP and Mg2+). Such modifications are known in the art. For instance, modification of the Mg2+ binding domain in helicases typically results in variants which do not function as helicases. These types of variants may act as molecular brakes (see below).

Over the entire length of the amino acid sequence of SEQ ID NO: 7, 3, 4, 5, 16, 8, 9, 10, 11, 12, 13, 14 or 15, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 7, 3, 4, 5, 16, 8, 9, 10, 11, 12, 13, 14 or 15 over the entire sequence. There may be at least 80%, for example at least 85%, 90% or 95%, amino acid identity over a stretch of 200 or more, for example 230, 250, 270, 280, 300, 400, 500, 600, 700, 800, 900 or 1000 or more, contiguous amino acids (“hard homology”). Homology is determined as described above. The variant may differ from the wild-type sequence in any of the ways discussed above with reference to SEQ ID NO: 1 above. The enzyme may be covalently attached to the pore. Any method may be used to covalently attach the enzyme to the pore.

A preferred molecular brake is TrwC Cba-Q594A (SEQ ID NO: 15 with the mutation Q594A). This variant does not function as a helicase (i.e. binds polynucleotides but does not move along them when provided with all the necessary components to facilitate movement, e.g. ATP and Mg2+).

In strand sequencing, the polynucleotide is translocated through the pore either with or against an applied potential. Exonucleases that act progressively or processively on double stranded polynucleotides can be used on the cis side of the pore to feed the remaining single strand through under an applied potential or the trans side under a reverse potential. Likewise, a helicase that unwinds the double stranded DNA can also be used in a similar manner. A polymerase may also be used. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential.

Any helicase may be used in the method. Helicases may work in two modes with respect to the pore. First, the method is preferably carried out using a helicase such that it moves the polynucleotide through the pore with the field resulting from the applied voltage. In this mode the 5′ end of the polynucleotide is first captured in the pore, and the helicase moves the polynucleotide into the pore such that it is passed through the pore with the field until it finally translocates through to the trans side of the membrane. Alternatively, the method is preferably carried out such that a helicase moves the polynucleotide through the pore against the field resulting from the applied voltage. In this mode the 3′ end of the polynucleotide is first captured in the pore, and the helicase moves the polynucleotide through the pore such that it is pulled out of the pore against the applied field until finally ejected back to the cis side of the membrane.

The method may also be carried out in the opposite direction. The 3′ end of the polynucleotide may be first captured in the pore and the helicase may move the polynucleotide into the pore such that it is passed through the pore with the field until it finally translocates through to the trans side of the membrane.

When the helicase is not provided with the necessary components to facilitate movement or is modified to hinder or prevent its movement, it can bind to the polynucleotide and act as a brake slowing the movement of the polynucleotide when it is pulled into the pore by the applied field. In the inactive mode, it does not matter whether the polynucleotide is captured either 3′ or 5′ down, it is the applied field which pulls the polynucleotide into the pore towards the trans side with the enzyme acting as a brake. When in the inactive mode, the movement control of the polynucleotide by the helicase can be described in a number of ways including ratcheting, sliding and braking. Helicase variants which lack helicase activity can also be used in this way.

The polynucleotide may be contacted with the polynucleotide binding protein and the pore in any order. It is preferred that, when the polynucleotide is contacted with the polynucleotide binding protein, such as a helicase, and the pore, the polynucleotide firstly forms a complex with the protein. When the voltage is applied across the pore, the polynucleotide/protein complex then forms a complex with the pore and controls the movement of the polynucleotide through the pore.

Any steps in the method using a polynucleotide binding protein are typically carried out in the presence of free nucleotides or free nucleotide analogues and an enzyme cofactor that facilitates the action of the polynucleotide binding protein. The free nucleotides may be one or more of any of the individual nucleotides discussed above. The free nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are preferably selected from AMP, TMP, GMP, CMP, UMP, dAMP, dTMP, dGMP or dCMP. The free nucleotides are preferably adenosine triphosphate (ATP). The enzyme cofactor is a factor that allows the construct to function. The enzyme cofactor is preferably a divalent metal cation. The divalent metal cation is preferably Mg2+, Mn2+, Ca2+ or Co2+. The enzyme cofactor is most preferably Mg2+.

### Helicase(s) and Molecular Brake(s)

The method may comprise providing the target analyte, particularly when the target analyte is a polynucleotide, with one or more helicases and one or more molecular brakes attached to the target polynucleotide. For example, the method of analyte characterisation may comprise:
(a) providing the polynucleotide with one or more helicases and one or more molecular brakes attached to the polynucleotide;
(b) contacting the polynucleotide with a pore of the invention and applying a potential across the pore such that the one or more helicases and the one or more molecular brakes are brought together and both control the movement of the polynucleotide with respect to, such as through, the pore;
(c) taking one or more measurements as the polynucleotide moves with respect to the pore wherein the measurements are indicative of one or more characteristics of the polynucleotide and thereby characterising the polynucleotide.

This type of method is discussed in detail in the International Application PCT/GB2014/052737.

The one or more helicases may be any of those discussed above. The one or more molecular brakes may be any compound or molecule which binds to the polynucleotide and slows the movement of the polynucleotide through the pore. The one or more molecular brakes preferably comprise one or more compounds which bind to the polynucleotide. The one or more compounds are preferably one or more macrocycles. Suitable macrocycles include, but are not limited to, cyclodextrins, calixarenes, cyclic peptides, crown ethers, cucurbiturils, pillararenes, derivatives thereof or a combination thereof. The cyclodextrin or derivative thereof may be any of those disclosed in Eliseev, A. V., and Schneider, H-J. (1994) J. Am. Chem. Soc. 116, 6081-6088. The agent is more preferably heptakis-6-amino-β-cyclodextrin (am7-βCD), 6-monodeoxy-6-monoamino-β-cyclodextrin (am1-βCD) or heptakis-(6-deoxy-6-guanidino)-cyclodextrin (gu7-βCD). The one or more molecular brakes are preferably one or more single stranded binding proteins (SSB). The one or more molecular brakes are more preferably a single-stranded binding protein (SSB) comprising a carboxy-terminal (C-terminal) region which does not have a net negative charge or (ii) a modified SSB comprising one or more modifications in its C-terminal region which decreases the net negative charge of the C-terminal region. The one or more molecular brakes are most preferably one of the SSBs disclosed in International Application No. PCT/GB2013/051924 (published as WO 2014/013259). The one or more molecular brakes are preferably one or more polynucleotide binding proteins. The polynucleotide binding protein may be any protein that is capable of binding to the polynucleotide and controlling its movement through the pore. It is straightforward in the art to determine whether or not a protein binds to a polynucleotide. The protein typically interacts with and modifies at least one property of the polynucleotide. The protein may modify the polynucleotide by cleaving it to form individual nucleotides or shorter chains of nucleotides, such as di- or trinucleotides. The moiety may modify the polynucleotide by orienting it or moving it to a specific position, i.e. controlling its movement.

The polynucleotide binding protein is preferably derived from a polynucleotide handling enzyme. The one or more molecular brakes may be derived from any of the polynucleotide handling enzymes discussed above. Modified versions of Phi29 polymerase (SEQ ID NO: 16) which act as molecular brakes are disclosed in U.S. Pat. No. 5,576,204. The one or more molecular brakes are preferably derived from a helicase. Any number of molecular brakes derived from a helicase may be used. For instance, 1, 2, 3, 4, 5, 6, 7, 8, 9, 10 or more helicases may be used as molecular brakes. If two or more helicases are be used as molecular brakes, the two or more helicases are typically the same helicase. The two or more helicases may be different helicases.

The two or more helicases may be any combination of the helicases mentioned above. The two or more helicases may be two or more Dda helicases. The two or more helicases may be one or more Dda helicases and one or more TrwC helicases. The two or more helicases may be different variants of the same helicase.

The two or more helicases are preferably attached to one another. The two or more helicases are more preferably covalently attached to one another. The helicases may be attached in any order and using any method. The one or more molecular brakes derived from helicases are preferably modified to reduce the size of an opening in the polynucleotide binding domain through which in at least one conformational state the polynucleotide can unbind from the helicase. This is disclosed in WO 2014/013260. Preferred helicase constructs for use in the invention are described in International Application Nos. PCT/GB2013/051925 (published as WO 2014/013260); PCT/GB2013/051924 (published as WO 2014/013259); PCT/GB2013/051928 (published as WO 2014/013262) and PCT/GB2014/052736.

If the one or more helicases are used in the active mode (i.e. when the one or more helicases are provided with all the necessary components to facilitate movement, e.g. ATP and Mg2+), the one or more molecular brakes are preferably (a) used in an inactive mode (i.e. are used in the absence of the necessary components to facilitate movement or are incapable of active movement), (b) used in an active mode where the one or more molecular brakes move in the opposite direction to the one or more helicases or (c) used in an active mode where the one or more molecular brakes move in the same direction as the one or more helicases and more slowly than the one or more helicases.

If the one or more helicases are used in the inactive mode (i.e. when the one or more helicases are not provided with all the necessary components to facilitate movement, e.g. ATP and Mg2+ or are incapable of active movement), the one or more molecular brakes are preferably (a) used in an inactive mode (i.e. are used in the absence of the necessary components to facilitate movement or are incapable of active movement) or (b) used in an active mode where the one or more molecular brakes move along the polynucleotide in the same direction as the polynucleotide through the pore.

The one or more helicases and one or more molecular brakes may be attached to the polynucleotide at any positions so that they are brought together and both control the movement of the polynucleotide through the pore. The one or more helicases and one or more molecular brakes are at least one nucleotide apart, such as at least 5, at least 10, at least 50, at least 100, at least 500, at least 1000, at least 5000, at least 10,000, at least 50,000 nucleotides or more apart. If the method concerns characterising a double stranded polynucleotide provided with a Y adaptor at one end and a hairpin loop adaptor at the other end, the one or more helicases are preferably attached to the Y adaptor and the one or more molecular brakes are preferably attached to the hairpin loop adaptor. In this embodiment, the one or more molecular brakes are preferably one or more helicases that are modified such that they bind the polynucleotide but do not function as a helicase. The one or more helicases attached to the Y adaptor are preferably stalled at a spacer as discussed in more detail below. The one or more molecular brakes attach to the hairpin loop adaptor are preferably not stalled at a spacer. The one or more helicases and the one or more molecular brakes are preferably brought together when the one or more helicases reach the hairpin loop. The one or more helicases may be attached to the Y adaptor before the Y adaptor is attached to the polynucleotide or after the Y adaptor is attached to the polynucleotide. The one or more molecular brakes may be attached to the hairpin loop adaptor before the hairpin loop adaptor is attached to the polynucleotide or after the hairpin loop adaptor is attached to the polynucleotide.

The one or more helicases and the one or more molecular brakes are preferably not attached to one another. The one or more helicases and the one or more molecular brakes are more preferably not covalently attached to one another. The one or more helicases and the one or more molecular brakes are preferably not attached as described in International Application Nos. PCT/GB2013/051925 (published as WO 2014/013260); PCT/GB2013/051924 (published as WO 2014/013259); PCT/GB2013/051928 (published as WO 2014/013262) and PCT/GB2014/052736.

### Spacers

The one or more helicases may be stalled at one or more spacers as discussed in International Application No. PCT/GB2014/050175. Any configuration of one or more helicases and one or more spacers disclosed in the International Application may be used in this invention.

When a part of the polynucleotide enters the pore and moves through the pore along the field resulting from the applied potential, the one or more helicases are moved past the spacer by the pore as the polynucleotide moves through the pore. This is because the polynucleotide (including the one or more spacers) moves through the pore and the one or more helicases remain on top of the pore.

The one or more spacers are preferably part of the polynucleotide, for instance they interrupt(s) the polynucleotide sequence. The one or more spacers are preferably not part of one or more blocking molecules, such as speed bumps, hybridised to the polynucleotide.

There may be any number of spacers in the polynucleotide, such as 1, 2, 3, 4, 5, 6, 7, 8, 9, 10 or more spacers. There are preferably two, four or six spacers in the polynucleotide. There may be one or more spacers in different regions of the polynucleotide, such as one or more spacers in the Y adaptor and/or hairpin loop adaptor.

The one or more spacers each provides an energy barrier which the one or more helicases cannot overcome even in the active mode. The one or more spacers may stall the one or more helicases by reducing the traction of the helicase (for instance by removing the bases from the nucleotides in the polynucleotide) or physically blocking movement of the one or more helicases (for instance using a bulky chemical group).

The one or more spacers may comprise any molecule or combination of molecules that stalls the one or more helicases. The one or more spacers may comprise any molecule or combination of molecules that prevents the one or more helicases from moving along the polynucleotide. It is straightforward to determine whether or not the one or more helicases are stalled at one or more spacers in the absence of a transmembrane pore and an applied potential. For instance, the ability of a helicase to move past a spacer and displace a complementary strand of DNA can be measured by PAGE.

The one or more spacers typically comprise a linear molecule, such as a polymer. The one or more spacers typically have a different structure from the polynucleotide. For instance, if the polynucleotide is DNA, the one or more spacers are typically not DNA. In particular, if the polynucleotide is deoxyribonucleic acid (DNA) or ribonucleic acid (RNA), the one or more spacers preferably comprise peptide nucleic acid (PNA), glycerol nucleic acid (GNA), threose nucleic acid (TNA), locked nucleic acid (LNA) or a synthetic polymer with nucleotide side chains. The one or more spacers may comprise one or more nucleotides in the opposite direction from the polynucleotide. For instance, the one or more spacers may comprise one or more nucleotides in the 3′ to 5′ direction when the polynucleotide is in the 5′ to 3′ direction. The nucleotides may be any of those discussed above.

The one or more spacers preferably comprises one or more nitroindoles, such as one or more 5-nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more iSpC3 groups (i.e. nucleotides which lack sugar and a base), one or more photo-cleavable (PC) groups, one or more hexandiol groups, one or more spacer 9 (iSp9) groups, one or more spacer 18 (iSp18) groups, a polymer or one or more thiol connections. The one or more spacers may comprise any combination of these groups. Many of these groups are commercially available from IDT® (Integrated DNA Technologies®).

The one or more spacers may contain any number of these groups. For instance, for 2-aminopurines, 2-6-diaminopurines, 5-bromo-deoxyuridines, inverted dTs, ddTs, ddCs, 5-methylcytidines, 5-hydroxymethylcytidines, 2′-O-Methyl RNA bases, Iso-dCs, Iso-dGs, iSpC3 groups, PC groups, hexandiol groups and thiol connections, the one or more spacers preferably comprise 2, 3, 4, 5, 6, 7, 8, 9, 10, 11, 12 or more. The one or more spacers preferably comprise 2, 3, 4, 5, 6, 7, 8 or more iSp9 groups. The one or more spacers preferably comprise 2, 3, 4, 5 or 6 or more iSp18 groups. The most preferred spacer is four iSp18 groups.

The polymer is preferably a polypeptide or a polyethylene glycol (PEG). The polypeptide preferably comprises 2, 3, 4, 5, 6, 7, 8, 9, 10, 11, 12 or more amino acids. The PEG preferably comprises 2, 3, 4, 5, 6, 7, 8, 9, 10, 11, 12 or more monomer units.

The one or more spacers preferably comprise one or more abasic nucleotides (i.e. nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic spacers can be inserted into polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). In one embodiment, the one or more spacers do not comprise any abasic nucleotides.

The one or more helicases may be stalled by (i.e. before) or on each linear molecule spacers. If linear molecule spacers are used, the polynucleotide is preferably provided with a double stranded region of polynucleotide adjacent to the end of each spacer past which the one or more helicases are to be moved. The double stranded region typically helps to stall the one or more helicases on the adjacent spacer. The presence of the double stranded region(s) is particularly preferred if the method is carried out at a salt concentration of about 100 mM or lower. Each double stranded region is typically at least 10, such as at least 12, nucleotides in length. If the polynucleotide used in the invention is single stranded, a double stranded region may be formed by hybridising a shorter polynucleotide to a region adjacent to a spacer. The shorter polynucleotide is typically formed from the same nucleotides as the polynucleotide, but may be formed from different nucleotides. For instance, the shorter polynucleotide may be formed from LNA.

If linear molecule spacers are used, the polynucleotide is preferably provided with a blocking molecule at the end of each spacer opposite to the end past which the one or more helicases are to be moved. This can help to ensure that the one or more helicases remain stalled on each spacer. It may also help retain the one or more helicases on the polynucleotide in the case that it/they diffuse(s) off in solution. The blocking molecule may be any of the chemical groups discussed below which physically cause the one or more helicases to stall. The blocking molecule may be a double stranded region of polynucleotide.

The one or more spacers preferably comprise one or more chemical groups which physically cause the one or more helicases to stall. The one or more chemical groups are preferably one or more pendant chemical groups. The one or more chemical groups may be attached to one or more nucleobases in the polynucleotide. The one or more chemical groups may be attached to the polynucleotide backbone. Any number of these chemical groups may be present, such as 2, 3, 4, 5, 6, 7, 8, 9, 10, 11, 12 or more. Suitable groups include, but are not limited to, fluorophores, streptavidin and/or biotin, cholesterol, methylene blue, dinitrophenols (DNPs), digoxigenin and/or anti-digoxigenin and dibenzylcyclooctyne groups.

Different spacers in the polynucleotide may comprise different stalling molecules. For instance, one spacer may comprise one of the linear molecules discussed above and another spacer may comprise one or more chemical groups which physically cause the one or more helicases to stall. A spacer may comprise any of the linear molecules discussed above and one or more chemical groups which physically cause the one or more helicases to stall, such as one or more abasics and a fluorophore.

Suitable spacers can be designed depending on the type of polynucleotide and the conditions under which the method of the invention is carried out. Most helicases bind and move along DNA and so may be stalled using anything that is not DNA. Suitable molecules are discussed above.

The method of the invention is preferably carried out in the presence of free nucleotides and/or the presence of a helicase cofactor. This is discussed in more detail below. In the absence of the transmembrane pore and an applied potential, the one or more spacers are preferably capable of stalling the one or more helicases in the presence of free nucleotides and/or the presence of a helicase cofactor.

If the method of the invention is carried out in the presence of free nucleotides and a helicase cofactor as discussed below (such that the one of more helicases are in the active mode), one or more longer spacers are typically used to ensure that the one or more helicases are stalled on the polynucleotide before they are contacted with the transmembrane pore and a potential is applied. One or more shorter spacers may be used in the absence of free nucleotides and a helicase cofactor (such that the one or more helicases are in the inactive mode).

The salt concentration also affects the ability of the one or more spacers to stall the one or more helicases. In the absence of the transmembrane pore and an applied potential, the one or more spacers are preferably capable of stalling the one or more helicases at a salt concentration of about 100 mM or lower. The higher the salt concentration used in the method of the invention, the shorter the one or more spacers that are typically used and vice versa.

Preferred combinations of features are shown in Table 3 below.

TABLE 3

| | Spacer | Spacer length | | Free | Helicase |
|---|---|---|---|---|---|
| Polynucleotide | composition* | (i.e. number of *) | Salt [ ] | nucleotides? | cofactor? |
| DNA | iSpC3 | 4 | 1M | Yes | Yes |
| DNA | iSp18 | 4 | 100-1000 | Yes | Yes |
| | | | mM | | |
| DNA | iSp18 | 6 | <100-1000 | Yes | Yes |
| | | | mM | | |
| DNA | iSp18 | 2 | 1M | Yes | Yes |
| DNA | iSpC3 | 12 | <100-1000 | Yes | Yes |
| | | | mM | | |
| DNA | iSpC3 | 20 | <100-1000 | Yes | Yes |
| | | | mM | | |
| DNA | iSp9 | 6 | 100-1000 | Yes | Yes |
| | | | mM | | |
| DNA | idSp | 4 | 1M | Yes | Yes |

The method may concern moving two or more helicases past a spacer. In such instances, the length of the spacer is typically increased to prevent the trailing helicase from pushing the leading helicase past the spacer in the absence of the pore and applied potential. If the method concerns moving two or more helicases past one or more spacers, the spacer lengths discussed above may be increased at least 1.5 fold, such 2 fold, 2.5 fold or 3 fold.

### Polypeptide Characterisation

The methods of the invention may also be utilised to characterise target polypeptides. Accordingly, the invention provides a method of characterising a target polypeptide, comprising:
(a) contacting the target polypeptide with a Cytotoxin K pore such that the target analyte moves with respect to the pore; and
(b) taking one or more measurements characteristic of the polypeptide as the polypeptide moves with respect to the pore,
thereby characterising the target polypeptide

The Cytotoxin K pore may be a wild type pore or a pore comprising a mutant CytK monomer of the invention described herein.

The method of polypeptide characterisation described herein may comprise: the invention may comprise (i) contacting the polypeptide with a polypeptide handling enzyme capable of controlling the movement of the polypeptide with respect to the pore; and (ii) taking one or more measurements characteristic of the polypeptide as the polypeptide moves with respect to the pore. Although, more preferably, wherein the method of characterising a target analyte comprises the characterising of a target polypeptide, the method preferably comprises forming a conjugate with a polynucleotide and using a polynucleotide-handling protein, such as a polynucleotide-handling enzyme to control the movement of the conjugate with respect to a nanopore. The methods of the present disclosure may also involve the control of the movement of a polypeptide with respect to a nanopore using a polypeptide-handling enzyme. Such methods involving the use of polypeptide- or polynucleotide-binding proteins are described in more detail in WO 2021/111125 and are applicable to methods of polypeptide characterisation involving the use of the mutant CytK monomers of the invention described herein.

The methods disclosed herein exploit the ability of polynucleotide-handling proteins to control the movement of conjugates which do not only comprise polynucleotides. In particular, conjugates which comprise polypeptides can be moved in a controlled manner using polynucleotide-handling proteins, as described herein. Polynucleotide-handling proteins suitable for use in the disclosed methods are described in more detail herein.

Accordingly, the method of characterising a target polypeptide preferably comprises:
conjugating the target polypeptide to a polynucleotide to form a polynucleotide-polypeptide conjugate;
contacting the conjugate with a polynucleotide-handling protein capable of controlling the movement of the polynucleotide with respect to a nanopore; and
taking one or more measurements characteristic of the polypeptide as the conjugate moves with respect to the nanopore,
   thereby characterising the polypeptide.

Any suitable polypeptide can be characterised using the methods disclosed herein. In some embodiments the target polypeptide is a protein or naturally occurring polypeptide. In some embodiments the polypeptide is a synthetic polypeptide. Polypeptides which can be characterised in accordance with the disclosed methods are described in more detail herein.

Any suitable polynucleotide can be used in forming the conjugate for use in the methods disclosed herein. In some embodiments the polynucleotide has a length at least as long as a portion of the target polypeptide to be characterised. In some embodiments the polynucleotide has a greater length than the portion of the target polypeptide to be characterised. This is discussed in more detail herein. Polynucleotides suitable for use in the disclosed methods are disclosed in more detail herein.

In the disclosed methods, the target polypeptide can be conjugated to the polynucleotide using any suitable means. Some exemplary means are described in more detail herein.

The conjugate formed in the disclosed methods is contacted with a polynucleotide-handling protein which is capable of controlling the movement of the polynucleotide with respect to a nanopore. Exemplary polynucleotide-handling proteins are described in more detail herein.

The polynucleotide-handling protein controls the movement of the polynucleotide with respect to a nanopore comprising a mutant CytK monomer of the invention. Any pore of the invention is suitable for use in the methods of polypeptide characterisation described herein.

The disclosed methods comprise taking one or more measurements characteristic of the polypeptide as the conjugate moves with respect to the nanopore. The one or more measurements can be any suitable measurements. Typically, the one or more measurements are electrical measurements, e.g. current measurements, and/or are one or more optical measurements. Apparatuses for recording suitable measurements, and the information that such measurements can provide, are described in more detail herein.

As disclosed herein, a polynucleotide can be used to control the movement of a polypeptide with respect to a nanopore comprising a CytK monomer of the invention. The movement of the polynucleotide is controlled by the polynucleotide-handling protein. Because the polynucleotide is conjugated to the polypeptide in the conjugate, the movement of the polynucleotide drives the movement of the polypeptide.

The use of a polynucleotide-handling protein to control the movement of the polynucleotide, and thus the movement of the polypeptide, may be associated with advantages compared to methods for characterising polypeptides known in the art. By way of example, polynucleotide-handling proteins are capable of processing the handling of polynucleotides with higher turnover rates compared to polypeptide-handling enzymes. This means that characterisation data may be obtained more rapidly for polypeptides characterised in accordance with the disclosed methods as compared to previously known methods.

These and other advantages will become apparent throughout the present disclosure.

The polynucleotide-handling protein is preferably located on the cis side of the nanopore and moves the conjugate into the pore, i.e. from the cis side to the trans side. The opposite setup could also be used.

In other words, in some embodiments, the polynucleotide-handling protein is located on the cis side of the nanopore and the polynucleotide-handling protein controls the movement of the conjugate from the cis side of the nanopore to the trans side of the nanopore. Thus, in some embodiments, the polynucleotide-handling protein is located on the cis side of the nanopore and the polynucleotide-handling protein controls the movement of the polynucleotide from the cis side of the nanopore to the trans side of the nanopore, thereby controlling the movement of the polypeptide through the nanopore.

In other embodiments, the polynucleotide-handling protein is located on the trans side of the nanopore and the polynucleotide-handling protein controls the movement of the conjugate from the trans side of the nanopore to the cis side of the nanopore. Thus, in some embodiments, the polynucleotide-handling protein is located on the trans side of the nanopore and the polynucleotide-handling protein controls the movement of the polynucleotide from the trans side of the nanopore to the cis side of the nanopore, thereby controlling the movement of the polypeptide through the nanopore.

As explained herein, the conjugate may comprise a leader. Any suitable leader may be used, as explained herein. Optionally, the leader may be a polynucleotide. The leader may be the same as the polynucleotide in the conjugate or may be different. As explained above, the leader may facilitate the threading of the conjugate through the nanopore.

In other words, in some embodiments the conjugate comprises one or more structures of the form L-{P-N}-Pₘ, wherein:
L is a leader, wherein L is optionally an N moiety;
P is a polypeptide;
N comprises a polynucleotide; and
m is 0 or 1;
   and the method may comprise threading the leader (L) through the nanopore thereby contacting the polypeptide (P) with the nanopore.

In some such embodiments, the polynucleotide-handling protein is located on the cis side of the nanopore and the method comprises allowing the polynucleotide-handling protein to control the movement of the polynucleotide moiety (N) from the cis side of the nanopore to the trans side of the nanopore, thereby controlling the movement of the polypeptide (P) through the nanopore. In other embodiments, the polynucleotide-handling protein is located on the trans side of the nanopore and the method comprises allowing the polynucleotide-handling protein to control the movement of the polynucleotide moiety (N) from the trans side of the nanopore to the cis side of the nanopore, thereby controlling the movement of the polypeptide (P) through the nanopore.

As explained in more detail herein, the conjugate may comprise one or more adapters and/or anchors.

As explained in more detail herein, in some embodiments the conjugate comprises multiple polynucleotides and polypeptides. In such embodiments the polynucleotide-handling protein sequentially controls the movement of the polynucleotides with respect to the nanopore, thus sequentially moving the polypeptide with respect to the nanopore. In this way, each polypeptide within the conjugate can be sequentially characterised in the disclosed methods.

For example, the conjugate may comprise one or more structures of the form L-P1-N-{P-N}ₙ-Pₘ, wherein:
n is a positive integer;
L is a leader, wherein L is optionally an N moiety;
each P, which may be the same or different, is a polypeptide;
each N, which may be the same or different, comprises a polynucleotide; and
m is 0 or 1;
   and the method may comprise threading the leader (L) through the nanopore thereby contacting polypeptide (P1) with the nanopore.

Typically, in such embodiments, n is from 1 to about 1000, e.g. from 2 to about 100, such as from about 3 to about 10, e.g. 1, 2, 3, 4, 5, 6, 7, 8, 9 or 10.

In some such embodiments, the polynucleotide-handling protein is located on the cis side of the nanopore and the method comprises allowing the polynucleotide-handling protein to control the movement of each polynucleotide (N) sequentially from the cis side of the nanopore to the trans side of the nanopore, thereby controlling the movement of each polypeptide (P) sequentially through the nanopore. In other such embodiments, the polynucleotide-handling protein is located on the trans side of the nanopore and the method comprises allowing the polynucleotide-handling protein to control the movement of each polynucleotide (N) sequentially from the trans side of the nanopore to the cis side of the nanopore, thereby controlling the movement of each polypeptide (P) sequentially through the nanopore.

Those skilled in the art will appreciate that when the conjugate comprises more than one polypeptide, it may be advantageous that (as described in more detail herein) the polynucleotide-handling protein can remain bound to the conjugate when it contacts the polypeptide without dissociating. This particularly allows polynucleotide-handling protein to pass over portions of polypeptide in the conjugate as it contacts them, in order to move onto sequential portions of polynucleotide in order to control the movement of the conjugate with respect to the nanopore.

A conjugate may comprise a polynucleotide and a polypeptide, and is contacted with a polynucleotide-handling protein such that the polypeptide threads the nanopore. In the illustrated embodiment a leader (which is optionally a further polynucleotide) is used to facilitate the threading of the polypeptide through the nanopore. Such use is within the scope of the disclosed methods, however this is not essential.

The polynucleotide-handling protein processes the polynucleotide conjugated to the polypeptide. As the polynucleotide-handling protein processes the polynucleotide, the conjugate is passed through the nanopore and so the polypeptide is passed through the nanopore. As the polypeptide is passed through the nanopore it is characterised.

The polynucleotide-handling protein may move the conjugate “out” of the pore, from the “viewpoint” of the polynucleotide-handling protein. For example, as shown the polynucleotide-handling protein is located on the cis side of the nanopore and moves the conjugate into the pore, i.e. from the trans side to the cis side. The opposite setup could also be used.

In other words, in some embodiments, the polynucleotide-handling protein is located on the cis side of the nanopore and the polynucleotide-handling protein controls the movement of the conjugate from the trans side of the nanopore to the cis side of the nanopore. Thus, in some embodiments the polynucleotide-handling protein is located on the cis side of the nanopore and the polynucleotide-handling protein controls the movement of the polynucleotide from the trans side of the nanopore to the cis side of the nanopore, thereby controlling the movement of the polypeptide through the nanopore.

In other embodiments, the polynucleotide-handling protein is located on the trans side of the nanopore and the polynucleotide-handling protein controls the movement of the conjugate from the cis side of the nanopore to the trans side of the nanopore. Thus, in some embodiments the polynucleotide-handling protein is located on the trans side of the nanopore and the polynucleotide-handling protein controls the movement of the polynucleotide from the cis side of the nanopore to the trans side of the nanopore, thereby controlling the movement of the polypeptide through the nanopore.

Using similar notation as above, in some embodiments the conjugate comprises one or more structures of the form L-{P-N}-Pₘ, wherein:
L is a leader, wherein L is optionally an N moiety;
P is a polypeptide;
N comprises a polynucleotide;
m is 0 or 1;
   and the method may comprise threading the leader (L) through the nanopore thereby contacting the polypeptide (P) with the nanopore.

In some such embodiments the polynucleotide-handling protein is located on the cis side of the nanopore and the method comprises allowing the polynucleotide-handling protein to control the movement of the polynucleotide (N) from the trans side of the nanopore to the cis side of the nanopore, thereby controlling the movement of the polypeptide (P) through the nanopore. In other such embodiments the polynucleotide-handling protein is located on the trans side of the nanopore and the method comprises allowing the polynucleotide-handling protein to control the movement of the polynucleotide (N) from the cis side of the nanopore to the trans side of the nanopore, thereby controlling the movement of the polypeptide (P) through the nanopore

In some embodiments, particularly embodiments where, as discussed above, the polynucleotide-handling protein controls the movement of the conjugate “out” of the nanopore, the conjugate may comprise a blocking moiety attached to the polypeptide via an optional linker. The blocking moiety is typically too large to pass through the nanopore and so when the movement of the conjugate with respect to the nanopore brings the blocking moiety into contact with the nanopore, the further movement of the conjugate through the nanopore is prevented. At such time the polynucleotide-handling protein may be allowed to transiently unbind from the conjugate. In embodiments of the disclosed methods in which the conjugate moves with respect to the nanopore under an applied force (e.g. a voltage potential or chemical potential) the conjugate may then move “back” through the pore in the opposite direction to the movement controlled by the polynucleotide-handling protein. The movement of the conjugate back through the pore allows the polypeptide portion of the conjugate to be re-characterised again.

The process can be repeated multiple times by sequentially allowing the polynucleotide-handling protein to bind and rebind to the conjugate. In such a manner, the conjugate may oscillate through the pore (i.e. it may be “flossed” through the nanopore). This “flossing” allows the polypeptide portion of the conjugate to be repeatedly characterised by the nanopore. In some embodiments this allows the accuracy of the characterisation information to be increased.

Any suitable blocking moiety can be used in such embodiments. For example, the conjugate may be modified with biotin and the blocking moiety may be e.g. streptavidin, avidin or neutravidin. The blocking moiety may be a large chemical group such as a dendrimer. The blocking moiety may be a nanoparticle or a bead. Other suitable blocking moieties will be apparent to those skilled in the art.

Accordingly, in some embodiments the method comprises
i) contacting the conjugate with the nanopore such that the blocking moiety is on the opposite side of the nanopore to the polynucleotide-handling protein;
ii) contacting the polynucleotide of the conjugate with the polynucleotide-handling protein;
iii) allowing the polynucleotide-handling protein to control the movement of the polynucleotide with respect to the nanopore thereby controlling the movement of the polypeptide through the nanopore;
iv) when the blocking moiety contacts the nanopore thereby preventing further movement of the conjugate through the nanopore, allowing the polynucleotide-handling protein to transiently unbind from the polynucleotide so that the conjugate moves through the nanopore under an applied force in a direction opposite to the direction of movement controlled by the polynucleotide-handling protein; and
v) optionally repeating steps (ii) to (iv) to oscillate the polypeptide through the nanopore.

### Polypeptide

As explained above, the disclosed methods may comprise characterising a target polypeptide within a conjugate as the conjugate moves with respect to a nanopore.

Any suitable polypeptide can be characterised in the disclosed methods.

In some embodiments the target polypeptide is an unmodified protein or a portion thereof, or a naturally occurring polypeptide or a portion thereof.

In some embodiments the target polypeptide is secreted from cells. Alternatively, the target polypeptide can be produced inside cells such that it must be extracted from cells for characterisation by the disclosed methods. The polypeptide may comprise the products of cellular expression of a plasmid, e.g. a plasmid used in cloning of proteins in accordance with the methods described in Sambrook et al., Molecular Cloning: A Laboratory Manual, 4th ed., Cold Spring Harbor Press, Plainsview, New York (2012); and Ausubel et al., Current Protocols in Molecular Biology (Supplement 114), John Wiley & Sons, New York (2016).

The polypeptide may be obtained from or extracted from any organism or microorganism. The polypeptide may be obtained from a human or animal, e.g. from urine, lymph, saliva, mucus, seminal fluid or amniotic fluid, or from whole blood, plasma or serum. The polypeptide may be obtained from a plant e.g. a cereal, legume, fruit or vegetable.

The target polypeptide can be provided as an impure mixture of one or more polypeptides and one or more impurities. Impurities may comprise truncated forms of the target polypeptide which are distinct from the “target polypeptides” for characterisation in the disclosed methods. For example, the target polypeptide may be a full length protein and impurities may comprise fractions of the protein. Impurities may also comprise proteins other than the target protein e.g. which may be co-purified from a cell culture or obtained from a sample.

A polypeptide may comprise any combination of any amino acids, amino acid analogs and modified amino acids (i.e. amino acid derivatives). Amino acids (and derivatives, analogs etc) in the polypeptide can be distinguished by their physical size and charge.

The amino acids/derivatives/analogs can be naturally occurring or artificial.

In some embodiments the polypeptide may comprise any naturally occurring amino acid. Twenty amino acids are encoded by the universal genetic code. These are alanine (A), arginine (R), asparagine (N), aspartic acid (D), cysteine (C), glutamic acid/glutamate (E), glutamine (Q), glycine (G), histidine (H), isoleucine (I), leucine (L), lysine (K), methionine (M), phenylalanine (F), proline (P), serine(S), threonine (T), tryptophan (W), tyrosine (Y) and valine (V). Other naturally occurring amino acids include selenocysteine and pyrrolysine.

In some embodiments the polypeptide is modified. In some embodiments the polypeptide is modified for detection using the disclosed methods. In some embodiments the disclosed methods are for characterising modifications in the target polypeptide.

In some embodiments one or more of the amino acids/derivatives/analogs in the polypeptide is modified. In some embodiments one or more of the amino acids/derivatives/analogs in the polypeptide is post-translationally modified. As such, the methods disclosed herein can be used to detect the presence, absence, number of positions of post-translational modifications in a polypeptide. The disclosed methods can be used to characterise the extent to which a polypeptide has been post-translationally modified.

Any one or more post-translational modifications may be present in the polypeptide. Typical post-translational modifications include modification with a hydrophobic group, modification with a cofactor, addition of a chemical group, glycation (the non-enzymatic attachment of a sugar), biotinylation and pegylation. Post-translational modifications can also be non-natural, such that they are chemical modifications done in the laboratory for biotechnological or biomedical purposes. This can allow monitoring the levels of the laboratory made peptide, polypeptide or protein in contrast to the natural counterparts.

Examples of post-translational modification with a hydrophobic group include myristoylation, attachment of myristate, a C14 saturated acid; palmitoylation, attachment of palmitate, a C16 saturated acid; isoprenylation or prenylation, the attachment of an isoprenoid group; farnesylation, the attachment of a farnesol group; geranylgeranylation, the attachment of a geranylgeraniol group; and glypiation, and glycosylphosphatidylinositol (GPI) anchor formation via an amide bond.

Examples of post-translational modification with a cofactor include lipoylation, attachment of a lipoate (C8) functional group; flavination, attachment of a flavin moiety (e.g. flavin mononucleotide (FMN) or flavin adenine dinucleotide (FAD)); attachment of heme C, for instance via a thioether bond with cysteine; phosphopantetheinylation, the attachment of a 4′-phosphopantetheinyl group; and retinylidene Schiff base formation.

Examples of post-translational modification by addition of a chemical group include acylation, e.g. O-acylation (esters), N-acylation (amides) or S-acylation (thioesters); acetylation, the attachment of an acetyl group for instance to the N-terminus or to lysine; formylation; alkylation, the addition of an alkyl group, such as methyl or ethyl; methylation, the addition of a methyl group for instance to lysine or arginine; amidation; butyrylation; gamma-carboxylation; glycosylation, the enzymatic attachment of a glycosyl group for instance to arginine, asparagine, cysteine, hydroxylysine, serine, threonine, tyrosine or tryptophan; polysialylation, the attachment of polysialic acid; malonylation; hydroxylation; iodination; bromination; citrulination; nucleotide addition, the attachment of any nucleotide such as any of those discussed above, ADP ribosylation; oxidation; phosphorylation, the attachment of a phosphate group for instance to serine, threonine or tyrosine (O-linked) or histidine (N-linked); adenylylation, the attachment of an adenylyl moiety for instance to tyrosine (O-linked) or to histidine or lysine (N-linked); propionylation; pyroglutamate formation; S-glutathionylation; Sumoylation; S-nitrosylation; succinylation, the attachment of a succinyl group for instance to lysine; selenoylation, the incorporation of selenium; and ubiquitinilation, the addition of ubiquitin subunits (N-linked).

It is within the scope of the methods provided herein that the polypeptide is labelled with a molecular label. A molecular label may be a modification to the polypeptide which promotes the detection of the polypeptide in the methods provided herein. For example the label may be a modification to the polypeptide which alters the signal obtained as conjugate is characterised. For example, the label may interfere with a flux of ions through the nanopore. In such a manner, the label may improve the sensitivity of the methods.

In some embodiments the polypeptide contains one or more cross-linked sections, e.g. C—C bridges. In some embodiments the polypeptides is not cross-linked prior to being characterised using the disclosed methods.

In some embodiments the polypeptide comprises sulphide-containing amino acids and thus has the potential to form disulphide bonds. Typically, in such embodiments, the polypeptide is reduced using a reagent such as DTT (Dithiothreitol) or TCEP (tris (2-carboxyethyl) phosphine) prior to being characterised using the disclosed methods.

In some embodiments the polypeptide is a full length protein or naturally occurring polypeptide. In some embodiments a protein or naturally occurring polypeptide is fragmented prior to conjugation to the polynucleotide. In some embodiments the protein or polypeptide is chemically or enzymatically fragmented. In some embodiments polypeptides or polypeptide fragments can be conjugated to form a longer target polypeptide.

The polypeptide can be a polypeptide of any suitable length. In some embodiments the polypeptide has a length of from about 2 to about 300 peptide units. In some embodiments the polypeptide has a length of from about 2 to about 100 peptide units, for example from about 2 to about 50 peptide units, e.g. from about 3 to about 50 peptide units, such as from about 5 to about 25 peptide units, e.g. from about 7 to about 16 peptide units, such as from about 9 to about 12 peptide units.

Any number of polypeptides can be characterised in the disclosed methods. For instance, the method may comprise characterising 2, 3, 4, 5, 6, 7, 8, 9, 10, 20, 30, 50, 100 or more polypeptides. If two or more polypeptides are used, they may be different polypeptides or two or more instances of the same polypeptide.

It will thus be apparent that the measurements taken in the disclosed methods are typically characteristic of one or more characteristics of the polypeptide selected from (i) the length of the polypeptide, (ii) the identity of the polypeptide, (iii) the sequence of the polypeptide, (iv) the secondary structure of the polypeptide and (v) whether or not the polypeptide is modified. In typical embodiments the measurements are characteristic of the sequence of the polypeptide or whether or not the polypeptide is modified, e.g. by one or more post-translational modifications. In some embodiments the measurements are characteristics of the sequence of the polypeptide.

In some embodiments the polypeptide is in a relaxed form. In some embodiments the polypeptide is held in a linearized form. Holding the polypeptide in a linearized form can facilitate the characterisation of the polypeptide on a residue-by-residue basis as “bunching up” of the polypeptide within the nanopore is prevented.

The polypeptide can be held in a linearized form using any suitable means.

For example, if the polypeptide is charged the polypeptide can be held in a linearized form by applying a voltage.

If the polypeptide is not charged or is only weakly charged then the charge can be altered or controlled by adjusting the pH. For example, the polypeptide can be held in a linearized form by using high pH to increase the relative negative charge of the polypeptide. Increasing the negative charge of the polypeptide allows it to be held in a linearized form under e.g. a positive voltage. Alternatively, the polypeptide can be held in a linearized form by using low pH to increase the relative positive charge of the polypeptide. Increasing the positive charge of the polypeptide allows it to be held in a linearized form under e.g. a negative voltage. In the disclosed methods a polynucleotide-handling protein is used to control the movement of a polynucleotide with respect to a nanopore. As a polynucleotide is typically negatively charged it is generally most suitable to increase the linearization of the polypeptide by increasing the pH thus making the polypeptide more negatively charged, in common with the polynucleotide. In this way, the conjugate retains an overall negative charge and thus can readily move e.g. under an applied voltage.

The polypeptide can be held in a linearized form by using suitable denaturing conditions. Suitable denaturing conditions include, for example, the presence of appropriate concentrations of denaturants such as guanidine HCl and/or urea. The concentration of such denaturants to use in the disclosed methods is dependent on the target polypeptide to be characterised in the methods and can be readily selected by those of skill in the art.

The polypeptide can be held in a linearized form by using suitable detergents. Suitable detergents for use in the disclosed methods include SDS (sodium dodecyl sulfate).

The polypeptide can be held in a linearized form by carrying out the disclosed methods at an elevated temperature. Increasing the temperature overcomes intra-strand bonding and allows the polypeptide to adopt a linearized form.

The polypeptide can be held in a linearized form by carrying out the disclosed methods under strong electro-osmotic forces. Such forces can be provided by using asymmetric salt conditions and/or providing suitable charge in the channel of the nanopore. The charge in the channel of a protein nanopore can be altered e.g. by mutagenesis. Altering the charge of a nanopore is well within the capacity of those skilled in the art. Altering the charge of a nanopore generates strong electro-osmotic forces from the unbalanced flow of cations and anions through the nanopore when a voltage potential is applied across the nanopore.

The polypeptide can be held in a linearized form by passing it through a structure such an array of nanopillars, through a nanoslit or across a nanogap. In some embodiments the physical constraints of such structures can force the polypeptide to adopt a linearized form.

### Formation of the Conjugate

As explained in more detail herein, the conjugate comprises a polynucleotide conjugated to the target polypeptide.

The target polypeptide can be conjugate to the polynucleotide at any suitable position. For example, the polypeptide can be conjugated to the polynucleotide at the N-terminus or the C-terminus of the polypeptide. The polypeptide can be conjugated to the polynucleotide via a side chain group of a residue (e.g. an amino acid residue) in the polypeptide.

In some embodiments the target polypeptide has a naturally occurring reactive functional group which can be used to facilitate conjugation to the polynucleotide. For example, a cysteine residue can be used to form a disulphide bond to the polynucleotide or to a modified group thereon.

In some embodiments the target polypeptide is modified in order to facilitate its conjugation to the polynucleotide. For example, in some embodiments the polypeptide is modified by attaching a moiety comprising a reactive functional group for attaching to the polynucleotide. For example, in some embodiments the polypeptide can be extended at the N-terminus or the C-terminus by one or more residues (e.g. amino acid residues) comprising one or more reactive functional groups for reacting with a corresponding reactive functional group on the polynucleotide. For example, in some embodiments the polypeptide can be extended at the N-terminus and/or the C-terminus by one or more cysteine residues. Such residues can be used for attachment to the polynucleotide portion of the conjugate, e.g. by maleimide chemistry (e.g. by reaction of cysteine with an azido-maleimide compound such as azido-[Pol]-maleimide wherein [Pol] is typically a short chain polymer such as PEG, e.g. PEG2, PEG3, or PEG4; followed by coupling to appropriately functionalised polynucleotide e.g. polynucleotide carrying a BCN group for reaction with the azide). Such chemistry is described in Example 2. For avoidance of doubt, when the polypeptide comprises an appropriate naturally occurring residue at the N- and/or C-terminus (e.g. a naturally occurring cysteine residue at the N- and/or C-terminus) then such residue(s) can be used for attachment to the polynucleotide.

In some embodiments a residue in the target polypeptide is modified to facilitate attachment of the target polypeptide to the polynucleotide. In some embodiments a residue (e.g. an amino acid residue) in the polypeptide is chemically modified for attachment to the polynucleotide. In some embodiments a residue (e.g. an amino acid residue) in the polypeptide is enzymatically modified for attachment to the polynucleotide.

The conjugation chemistry between the polynucleotide and the polypeptide in the conjugate is not particularly limited. Any suitable combination of reactive functional groups can be used. Many suitable reactive groups and their chemical targets are known in the art. Some exemplary reactive groups and their corresponding targets include aryl azides which may react with amine, carbodiimides which may react with amines and carboxyl groups, hydrazides which may react with carbohydrates, hydroxmethyl phosphines which may react with amines, imidoesters which may react with amines, isocyanates which may react with hydroxyl groups, carbonyls which may react with hydrazines, maleimides which may react with sulfhydryl groups, NHS-esters which may react with amines, PFP-esters which may react with amines, psoralens which may react with thymine, pyridyl disulfides which may react with sulfhydryl groups, vinyl sulfones which may react with sulfhydryl amines and hydroxyl groups, vinylsulfonamides, and the like.

Other suitable chemistry for conjugating the polypeptide to the polynucleotide includes click chemistry. Many suitable click chemistry reagents are known in the art. Suitable examples of click chemistry include, but are not limited to, the following:
(a) copper (I)-catalyzed azide-alkyne cycloadditions (azide alkyne Huisgen cycloadditions);
(b) strain-promoted azide-alkyne cycloadditions; including alkene and azide [3+2] cycloadditions; alkene and tetrazine inverse-demand Diels-Alder reactions; and alkene and tetrazole photoclick reactions;
(c) copper-free variant of the 1,3 dipolar cycloaddition reaction, where an azide reacts with an alkyne under strain, for example in a cyclooctane ring such as in bicycle[6.1.0]nonyne (BCN);
(d) the reaction of an oxygen nucleophile on one linker with an epoxide or aziridine reactive moiety on the other; and
(e) the Staudinger ligation, where the alkyne moiety can be replaced by an aryl phosphine, resulting in a specific reaction with the azide to give an amide bond.

Any reactive group may be used to form the conjugate. Some suitable reactive groups include [1,4-Bis[3-(2-pyridyldithio)propionamido]butane; 1,1 1-bis-maleimidotriethyleneglycol; 3,3′-dithiodipropionic acid di(N-hydroxysuccinimide ester); ethylene glycol-bis(succinic acid N-hydroxysuccinimide ester); 4,4′-diisothiocyanatostilbene-2,2′-disulfonic acid disodium salt; Bis[2-(4-azidosalicylamido)ethyl]disulphide; 3-(2-pyridyldithio) propionic acid N-hydroxysuccinimide ester; 4-maleimidobutyric acid N-hydroxysuccinimide ester; Iodoacetic acid N-hydroxysuccinimide ester; S-acetylthioglycolic acid N-hydroxysuccinimide ester; azide-PEG-maleimide; and alkyne-PEG-maleimide. The reactive group may be any of those disclosed in WO 2010/086602, particularly in Table 3 of that application.

In some embodiments the reactive functional group is comprised in the polynucleotide and the target functional group is comprised in the polypeptide prior to the conjugation step. In other embodiments the reactive functional group is comprised in the polypeptide and the target functional group is comprised in the polynucleotide prior to the conjugation step. In some embodiments the reactive functional group is attached directly to the polypeptide. In some embodiments the reactive functional group is attached to the polypeptide via a spacer. Any suitable spacer can be used. Suitable spacers include for example alkyl diamines such as ethyl diamine, etc.

As will be apparent from the above discussed, in some embodiments the conjugate comprises a plurality of polypeptide sections and/or a plurality of polynucleotide sections. For example the conjugate may comprise a structure of the form . . . P—N-P-N-P-N . . . wherein P is a polypeptide and N is a polynucleotide. In such embodiments the polynucleotide-handling protein sequentially controls the N portions of the conjugate with respect to the nanopore and thus sequentially controls the movement of the P sections with respect to the nanopore, thus allowing the sequential characterisation of the P sections. In such embodiments the plurality of polynucleotides and polypeptides may be conjugated together by the same or different chemistries.

As explained herein, the conjugate may comprise a leader. Any suitable leader may be used, as explained herein. In some embodiments the leader is a polynucleotide. In embodiments wherein the leader is a polynucleotide the leader may be the same sort of polynucleotide as the polynucleotide used in the conjugate, or it may be a different type of polynucleotide. For example, the polynucleotide in the conjugate may be DNA and the leader may be RNA or vice versa.

In some embodiments the leader is a charged polymer, e.g. a negatively charged polymer. In some embodiments the leader comprises a polymer such as PEG or a polysaccharide. In such embodiments the leader may be from 10 to 150 monomer units (e.g. ethylene glycol or saccharide units) in length, such as from 20 to 120, e.g. 30 to 100, for example 40 to 80 such as 50 to 70 monomer units (e.g. ethylene glycol or saccharide units) in length.

The disclosed methods of characterising a target polypeptide described herein may comprise conjugating a polypeptide to a polynucleotide and controlling the movement of the conjugate with respect to a nanopore using a polynucleotide-handling protein.

In the disclosed methods, any suitable polynucleotide can be used. Such polynucleotides are described further herein in relation to methods of polynucleotide characterisation.

### Coupling

The target analyte, preferably wherein the analyte is a polynucleotide or polypeptide, is may be coupled to the membrane comprising the pore in the method of the invention described herein. The method may comprise coupling the analyte to the membrane comprising the pore. The polynucleotide is preferably coupled to the membrane using one or more anchors. The polynucleotide may be coupled to the membrane using any known method.

Each anchor comprises a group which couples (or binds) to the analyte and a group which couples (or binds) to the membrane. Each anchor may covalently couple (or bind) to the analyte and/or the membrane.

If the analyte is a polynucleotide, a Y adaptor and/or a hairpin loop adaptors (both of such adaptors are known in the art) may be used, and the polynucleotide is preferably coupled to the membrane using the adaptor(s).

The analyte may be coupled to the membrane using any number of anchors, such as 2, 3, 4 or more anchors. For instance, a analyte may be coupled to the membrane using two anchors each of which separately couples (or binds) to both the analyte and membrane.

The one or more anchors may comprise one or more helicases and/or one or more molecular brakes.

If the membrane is an amphiphilic layer, such as a copolymer membrane or a lipid bilayer, the one or more anchors preferably comprise a polypeptide anchor present in the membrane and/or a hydrophobic anchor present in the membrane. The hydrophobic anchor is preferably a lipid, fatty acid, sterol, carbon nanotube, polypeptide, protein or amino acid, for example cholesterol, palmitate, tocopherol, or a charge-neutralized alkyl-phosporothioate. In preferred embodiments, the one or more anchors are not the pore.

The components of the membrane, such as the amphiphilic molecules, copolymer or lipids, may be chemically-modified or functionalised to form the one or more anchors. Examples of suitable chemical modifications and suitable ways of functionalising the components of the membrane are discussed in more detail below. Any proportion of the membrane components may be functionalised, for example at least 0.01%, at least 0.1%, at least 1%, at least 10%, at least 25%, at least 50% or 100%.

The analyte may be coupled directly to the membrane. The one or more anchors used to couple the analyte to the membrane preferably comprise a linker. The one or more anchors may comprise one or more, such as 2, 3, 4 or more, linkers. One linker may be used couple more than one, such as 2, 3, 4 or more, analytes to the membrane.

Preferred linkers include, but are not limited to, polymers, such as polynucleotides, polyethylene glycols (PEGs), polysaccharides and polypeptides. These linkers may be linear, branched or circular. For instance, the linker may be a circular polynucleotide. The polynucleotide may hybridise to a complementary sequence on the circular polynucleotide linker.

The one or more anchors or one or more linkers may comprise a component that can be cut to broken down, such as a restriction site or a photolabile group.

Functionalised linkers and the ways in which they can couple molecules are known in the art. For instance, linkers functionalised with maleimide groups will react with and attach to cysteine residues in proteins. In the context of this invention, the protein may be present in the membrane or may be used to couple (or bind) to the analyte. This is discussed in more detail below.

Crosslinkage of analyte can be avoided using a “lock and key” arrangement. Only one end of each linker may react together to form a longer linker and the other ends of the linker each react with the polynucleotide or membrane respectively. Such linkers are described in International Application No. PCT/GB10/000132 (published as WO 2010/086602).

The use of a linker is preferred in the sequencing embodiments discussed herein. If a polynucleotide or polypeptide is permanently coupled directly to the membrane in the sense that it does not uncouple when interacting with the pore (i.e. does not uncouple in step (b) or (e)), then some sequence data will be lost as the sequencing run cannot continue to the end of the analyte due to the distance between the membrane and the pore. If a linker is used, then the polynucleotide or polypeptide can be processed to completion.

The coupling may be permanent or stable. In other words, the coupling may be such that the analyte remains coupled to the membrane when interacting with the pore.

The coupling may be transient. In other words, the coupling may be such that the polynucleotide may decouple from the membrane when interacting with the pore.

For certain applications, such as aptamer detection, the transient nature of the coupling is preferred. If, for example, a permanent or stable linker is attached directly to either the 5′ or 3′ end of a polynucleotide target analyte and the linker is shorter than the distance between the membrane and the transmembrane pore's channel, then some sequence data will be lost as the sequencing run cannot continue to the end of the polynucleotide. If the coupling is transient, then when the coupled end randomly becomes free of the membrane, then the polynucleotide can be processed to completion. Chemical groups that form permanent/stable or transient links are discussed in more detail below. The polynucleotide may be transiently coupled to an amphiphilic layer or triblock copolymer membrane using cholesterol or a fatty acyl chain. Any fatty acyl chain having a length of from 6 to 30 carbon atom, such as hexadecanoic acid, may be used.

In preferred embodiments, a target analyte, such as a polypeptide or polynucleotide, is coupled to an amphiphilic layer such as a triblock copolymer membrane or lipid bilayer. Coupling of nucleic acids to synthetic lipid bilayers has been carried out previously with various different tethering strategies. These are summarised in Table 4 below.

TABLE 4

| Anchor | Type of | |
|---|---|---|
| comprising | coupling | Reference |
| Thiol | Stable | Yoshina-Ishii, C. and S. G. Boxer (2003). |
| | | “Arrays of mobile tethered vesicles on |
| | | supported lipid bilayers.” J Am Chem Soc |
| | | 125(13): 3696-7. |
| Biotin | Stable | Nikolov, V., R. Lipowsky, et al. (2007). |
| | | “Behavior of giant vesicles with |
| | | anchored DNA molecules.” Biophys J |
| | | 92(12): 4356-68 |
| Cholesterol | Transient | Pfeiffer, I. and F. Hook (2004). “Bivalent |
| | | cholesterol-based coupling of |
| | | oligonucletides to lipid membrane |
| | | assemblies.” J Am Chem Soc 126(33): |
| | | 10224-5 |
| Surfactant | Stable | van Lengerich, B., R. J. Rawle, et al. |
| (e.g. Lipid, | | “Covalent attachment of lipid vesicles to a |
| Palmitate, etc) | | fluid-supported bilayer allows observation |
| | | of DNA-mediated vesicle interactions.” |
| | | Langmuir 26(11): 8666-72 |
| Charge- | Transient | Jones, S., et al. “Hydrophobic Interaction |
| neutralized | | between DNA Duplexes and Synthetic and |
| alkyl- | | Biological Membranes.” J Am Chem Soc |
| phosphorothioate | | 143(22): 8305-8313 |
| (PPT) belt | | |

Synthetic polynucleotides and/or linkers may be functionalised using a modified phosphoramidite in the synthesis reaction, which is easily compatible for the direct addition of suitable anchoring groups, such as cholesterol, tocopherol, palmitate, thiol, lipid and biotin groups. These different attachment chemistries give a suite of options for attachment to polynucleotides. Each different modification group couples the polynucleotide in a slightly different way and coupling is not always permanent so giving different dwell times for the polynucleotide to the membrane. The advantages of transient coupling are discussed above.

Coupling of polynucleotides to a linker or to a functionalised membrane can also be achieved by a number of other means provided that a complementary reactive group or an anchoring group can be added to the polynucleotide. The addition of reactive groups to either end of a polynucleotide has been reported previously. A thiol group can be added to the 5′ of ssDNA or dsDNA using T4 polynucleotide kinase and ATPYS (Grant, G. P. and P. Z. Qin (2007). “A facile method for attaching nitroxide spin labels at the 5′ terminus of nucleic acids.” Nucleic Acids Res 35 (10): e77). An azide group can be added to the 5′-phosphate of ssDNA or dsDNA using T4 polynucleotide kinase and γ-[2-Azidoethyl]-ATP or γ-[6-Azidohexyl]-ATP. Using thiol or Click chemistry a tether, containing either a thiol, iodoacetamide OPSS or maleimide group (reactive to thiols) or a DIBO (dibenzocyclooxtyne) or alkyne group (reactive to azides), can be covalently attached to the polynucleotide. A more diverse selection of chemical groups, such as biotin, thiols and fluorophores, can be added using terminal transferase to incorporate modified oligonucleotides to the 3′ of ssDNA (Kumar, A., P. Tchen, et al. (1988). “Nonradioactive labeling of synthetic oligonucleotide probes with terminal deoxynucleotidyl transferase.” Anal Biochem 169 (2): 376-82). Streptavidin/biotin and/or streptavidin/desthiobiotin coupling may be used for any other polynucleotide. The Examples below describes how a polynucleotide can be coupled to a membrane using streptavidin/biotin and streptavidin/desthiobiotin. It may also be possible that anchors may be directly added to polynucleotides using terminal transferase with suitably modified nucleotides (e.g. cholesterol or palmitate).

The one or more anchors preferably couple a polynucleotide target analyte to the membrane via hybridisation. Hybridisation in the one or more anchors allows coupling in a transient manner as discussed above. The hybridisation may be present in any part of the one or more anchors, such as between the one or more anchors and the polynucleotide, within the one or more anchors or between the one or more anchors and the membrane. For instance, a linker may comprise two or more polynucleotides, such as 3, 4 or 5 polynucleotides, hybridised together. The one or more anchors may hybridise to the polynucleotide. The one or more anchors may hybridise directly to the polynucleotide or directly to a Y adaptor and/or leader sequence attached to the polynucleotide or directly to a hairpin loop adaptor attached to the polynucleotide (as discussed below). Alternatively, the one or more anchors may be hybridised to one or more, such as 2 or 3, intermediate polynucleotides (or “splints”) which are hybridised to the polynucleotide, to a Y adaptor and/or leader sequence attached to the polynucleotide or to a hairpin loop adaptor attached to the polynucleotide (as discussed below).

The one or more anchors may comprise a single stranded or double stranded polynucleotide. One part of the anchor may be ligated to a single stranded or double stranded polynucleotide. Ligation of short pieces of ssDNA have been reported using T4 RNA ligase I (Troutt, A. B., M. G. McHeyzer-Williams, et al. (1992). “Ligation-anchored PCR: a simple amplification technique with single-sided specificity.” Proc Natl Acad Sci USA 89 (20): 9823-5). Alternatively, either a single stranded or double stranded polynucleotide can be ligated to a double stranded polynucleotide and then the two strands separated by thermal or chemical denaturation. To a double stranded polynucleotide, it is possible to add either a piece of single stranded polynucleotide to one or both of the ends of the duplex, or a double stranded polynucleotide to one or both ends. For addition of single stranded polynucleotides to the a double stranded polynucleotide, this can be achieved using T4 RNA ligase I as for ligation to other regions of single stranded polynucleotides. For addition of double stranded polynucleotides to a double stranded polynucleotide then ligation can be “blunt-ended”, with complementary 3′ dA/dT tails on the polynucleotide and added polynucleotide respectively (as is routinely done for many sample prep applications to prevent concatemer or dimer formation) or using “sticky-ends” generated by restriction digestion of the polynucleotide and ligation of compatible adapters. Then, when the duplex is melted, each single strand will have either a 5′ or 3′ modification if a single stranded polynucleotide was used for ligation or a modification at the 5′ end, the 3′ end or both if a double stranded polynucleotide was used for ligation.

If the polynucleotide is a synthetic strand, the one or more anchors can be incorporated during the chemical synthesis of the polynucleotide. For instance, the polynucleotide can be synthesised using a primer having a reactive group attached to it. Adenylated polynucleotides are intermediates in ligation reactions, where an adenosine-monophosphate is attached to the 5′-phosphate of the polynucleotide. Various kits are available for generation of this intermediate, such as the 5′ DNA Adenylation Kit from NEB. By substituting ATP in the reaction for a modified nucleotide triphosphate, then addition of reactive groups (such as thiols, amines, biotin, azides, etc) to the 5′ of a polynucleotide can be possible. It may also be possible that anchors could be directly added to polynucleotides using a 5′ DNA adenylation kit with suitably modified nucleotides (e.g. cholesterol or palmitate).

A common technique for the amplification of sections of genomic DNA is using polymerase chain reaction (PCR). Here, using two synthetic oligonucleotide primers, a number of copies of the same section of DNA can be generated, where for each copy the 5′ of each strand in the duplex will be a synthetic polynucleotide. Single or multiple nucleotides can be added to 3′ end of single or double stranded DNA by employing a polymerase. Examples of polymerases which could be used include, but are not limited to, Terminal Transferase, Klenow and E. coli Poly (A) polymerase). By substituting ATP in the reaction for a modified nucleotide triphosphate then anchors, such as a cholesterol, thiol, amine, azide, biotin or lipid, can be incorporated into double stranded polynucleotides. Therefore, each copy of the amplified polynucleotide will contain an anchor.

Ideally, the polynucleotide is coupled to the membrane without having to functionalise the polynucleotide. This can be achieved by coupling the one or more anchors, such as a polynucleotide binding protein or a chemical group, to the membrane and allowing the one or more anchors to interact with the polynucleotide or by functionalising the membrane. The one or more anchors may be coupled to the membrane by any of the methods described herein. In particular, the one or more anchors may comprise one or more linkers, such as maleimide functionalised linkers.

In this embodiment, the polynucleotide is typically RNA, DNA, PNA, TNA or LNA and may be double or single stranded. This embodiment is particularly suited to genomic DNA polynucleotides.

The one or more anchors can comprise any group that couples to, binds to or interacts with single or double stranded polynucleotides, specific nucleotide sequences within the polynucleotide or patterns of modified nucleotides within the polynucleotide, or any other ligand that is present on the polynucleotide.

Suitable binding proteins for use in anchors include, but are not limited to, E. coli single stranded binding protein, P5 single stranded binding protein, T4 gp32 single stranded binding protein, the TOPO V dsDNA binding region, human histone proteins, E. coli HU DNA binding protein and other archaeal, prokaryotic or eukaryotic single stranded or double stranded polynucleotide (or nucleic acid) binding proteins, including those listed below.

The specific nucleotide sequences could be sequences recognised by transcription factors, ribosomes, endonucleases, topoisomerases or replication initiation factors. The patterns of modified nucleotides could be patterns of methylation or damage.

The one or more anchors can comprise any group which couples to, binds to, intercalates with or interacts with a polynucleotide. The group may intercalate or interact with the polynucleotide via electrostatic, hydrogen bonding or Van der Waals interactions. Such groups include a lysine monomer, poly-lysine (which will interact with ssDNA or dsDNA), ethidium bromide (which will intercalate with dsDNA), universal bases or universal nucleotides (which can hybridise with any polynucleotide) and osmium complexes (which can react to methylated bases). A polynucleotide may therefore be coupled to the membrane using one or more universal nucleotides attached to the membrane. Each universal nucleotide may be coupled to the membrane using one or more linkers. The universal nucleotide preferably comprises one of the following nucleobases: hypoxanthine, 4-nitroindole, 5-nitroindole, 6-nitroindole, formylindole, 3-nitropyrrole, nitroimidazole, 4-nitropyrazole, 4-nitrobenzimidazole, 5-nitroindazole, 4-aminobenzimidazole or phenyl (C6-aromatic ring). The universal nucleotide more preferably comprises one of the following nucleosides: 2′-deoxyinosine, inosine, 7-deaza-2′-deoxyinosine, 7-deaza-inosine, 2-aza-deoxyinosine, 2-aza-inosine, 2-O′-methylinosine, 4-nitroindole 2′-deoxyribonucleoside, 4-nitroindole ribonucleoside, 5-nitroindole 2′ deoxyribonucleoside, 5-nitroindole ribonucleoside, 6-nitroindole 2′ deoxyribonucleoside, 6-nitroindole ribonucleoside, 3-nitropyrrole 2′ deoxyribonucleoside, 3-nitropyrrole ribonucleoside, an acyclic sugar analogue of hypoxanthine, nitroimidazole 2′ deoxyribonucleoside, nitroimidazole ribonucleoside, 4-nitropyrazole 2′ deoxyribonucleoside, 4-nitropyrazole ribonucleoside, 4-nitrobenzimidazole 2′ deoxyribonucleoside, 4-nitrobenzimidazole ribonucleoside, 5-nitroindazole 2′ deoxyribonucleoside, 5-nitroindazole ribonucleoside, 4-aminobenzimidazole 2′ deoxyribonucleoside, 4-aminobenzimidazole ribonucleoside, phenyl C-ribonucleoside, phenyl C-2′-deoxyribosyl nucleoside, 2′-deoxynebularine, 2′-deoxyisoguanosine, K-2′-deoxyribose, P-2′-deoxyribose and pyrrolidine. The universal nucleotide more preferably comprises 2′-deoxyinosine. The universal nucleotide is more preferably IMP or dIMP. The universal nucleotide is most preferably dPMP (2′-Deoxy-P-nucleoside monophosphate) or dKMP (N6-methoxy-2,6-diaminopurine monophosphate).

The one or more anchors may couple to (or bind to) the polynucleotide via Hoogsteen hydrogen bonds (where two nucleobases are held together by hydrogen bonds) or reversed Hoogsteen hydrogen bonds (where one nucleobase is rotated through 180° with respect to the other nucleobase). For instance, the one or more anchors may comprise one or more nucleotides, one or more oligonucleotides or one or more polynucleotides which form Hoogsteen hydrogen bonds or reversed Hoogsteen hydrogen bonds with the polynucleotide. These types of hydrogen bonds allow a third polynucleotide strand to wind around a double stranded helix and form a triplex. The one or more anchors may couple to (or bind to) a double stranded polynucleotide by forming a triplex with the double stranded duplex.

In this embodiment at least 1%, at least 10%, at least 25%, at least 50% or 100% of the membrane components may be functionalised.

Where the one or more anchors comprise a protein, they may be able to anchor directly into the membrane without further functonalisation, for example if it already has an external hydrophobic region which is compatible with the membrane. Examples of such proteins include, but are not limited to, transmembrane proteins, intramembrane proteins and membrane proteins. Alternatively the protein may be expressed with a genetically fused hydrophobic region which is compatible with the membrane. Such hydrophobic protein regions are known in the art.

The one or more anchors are preferably mixed with the polynucleotide before contacting with the membrane, but the one or more anchors may be contacted with the membrane and subsequently contacted with the polynucleotide.

In another aspect the polynucleotide may be functionalised, using methods described above, so that it can be recognised by a specific binding group. Specifically the analyte may be functionalised with a ligand such as biotin (for binding to streptavidin), amylose (for binding to maltose binding protein or a fusion protein), Ni-NTA (for binding to poly-histidine or poly-histidine tagged proteins) or a peptides (such as an antigen).

According to a preferred embodiment, the one or more anchors may be used to couple a polynucleotide to the membrane when the polynucleotide is attached to a leader sequence which preferentially threads into the pore. Leader sequences are discussed in more detail below. Preferably, the polynucleotide is attached (such as ligated) to a leader sequence which preferentially threads into the pore. Such a leader sequence may comprise a homopolymeric polynucleotide or an abasic region. The leader sequence is typically designed to hybridise to the one or more anchors either directly or via one or more intermediate polynucleotides (or splints). In such instances, the one or more anchors typically comprise a polynucleotide sequence which is complementary to a sequence in the leader sequence or a sequence in the one or more intermediate polynucleotides (or splints). In such instances, the one or more splints typically comprise a polynucleotide sequence which is complementary to a sequence in the leader sequence.

An example of a molecule used in chemical attachment is EDC (1-ethyl-3-[3-dimethylaminopropyl]carbodiimide hydrochloride). Reactive groups can also be added to the 5′ of polynucleotides using commercially available kits (Thermo Pierce, Part No. 22980). Suitable methods include, but are not limited to, transient affinity attachment using histidine residues and Ni-NTA, as well as more robust covalent attachment by reactive cysteines, lysines or non natural amino acids.

### Kit

Also provided is a kit comprising:
a pore according to the invention; and
a polynucleotide binding protein or polypeptide handling enzyme.

In some embodiments, said pore is modified to alter the ability of the monomer to interact with an analyte in accordance with variants described herein. Most preferably, one or more constrictions in the pore are modified in accordance with the variants described herein, thereby altering the ability of the one or more constrictions to interact with an analyte.

The kit may be configured for use with an algorithm, also provided herein, adapted to be run on a computer system. The algorithm may be adapted to detect information characteristic of a polypeptide (e.g. characteristic of the sequence of the polypeptide and/or whether the polypeptide is modified), and to selectively process the signal obtained as a conjugate comprising the polypeptide conjugated to a polynucleotide moves with respect to the nanopore. Also provided is a system comprising computing means configured to detect information characteristic of a polypeptide (e.g. characteristic of the sequence of the polypeptide and/or whether the polypeptide is modified) and to selectively process the signal obtained as a conjugate comprising the polypeptide conjugated to a polynucleotide moves with respect to the nanopore. In some embodiments the system comprises receiving means for receiving data from detection of the polypeptide, processing means for processing the signal obtained as the conjugate moves with respect to the nanopore, and output means for outputting the characterisation information thus obtained.

It is to be understood that although particular embodiments, specific configurations as well as materials and/or molecules, have been discussed herein for methods according to the present invention, various changes or modifications in form and detail may be made without departing from the scope and spirit of this invention. The preceding embodiments and following examples are provided for illustration only, and should not be considered limiting the application. The application is limited only by the claims.

### Examples

### Example—Materials and Methods

Experimental results are described in detail in the Figure legends.

### Computational Tools

Pairwise sequence alignment (see particularly FIG. 1) was performed using publicly available software, Clustalx (http://www.clustal.org/clustal2/).

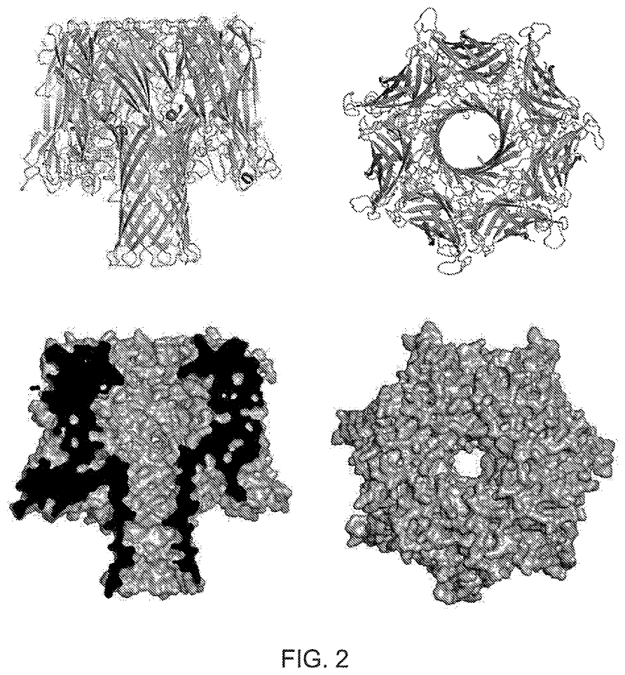

A structural model of CytK (see particularly FIG. 2) was made using the Modeller software (https://salilab.org/modeller/).

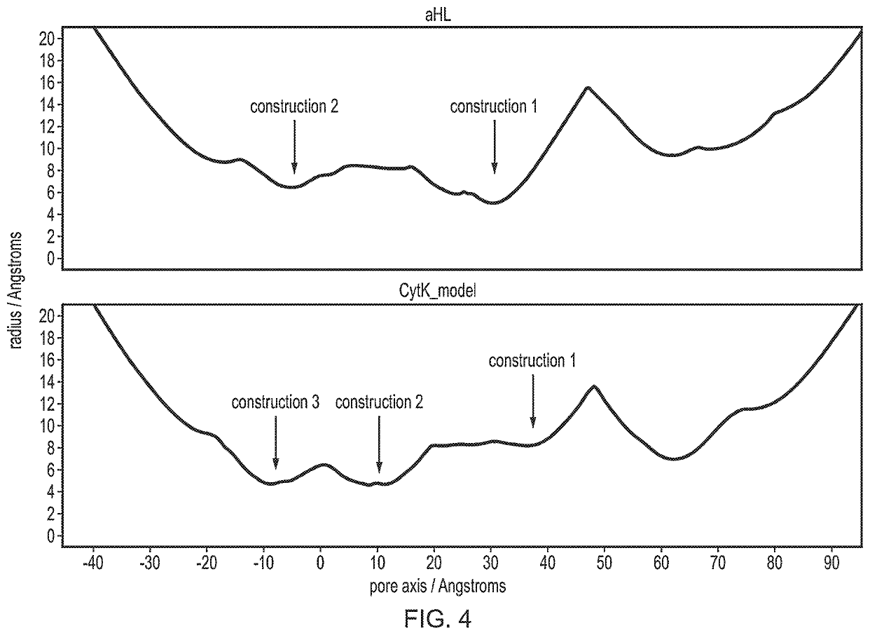

Pore radial profiles (see particularly FIG. 4) were generated using the publicly available software, HOLE (http://www.holeprogram.org/).

E coli Pore Production

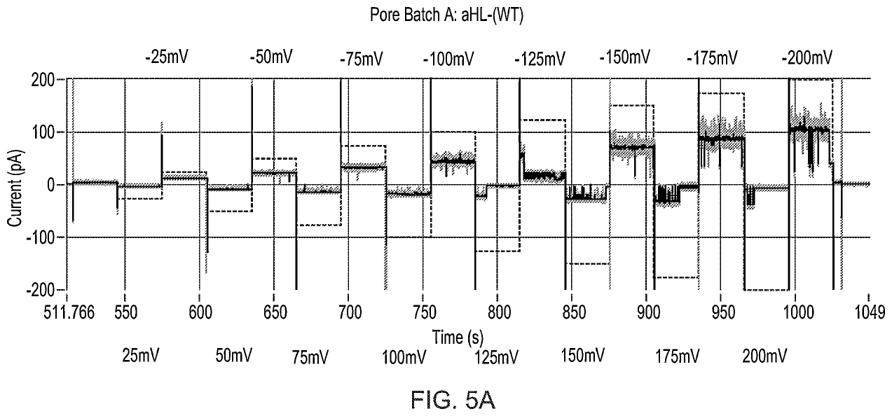

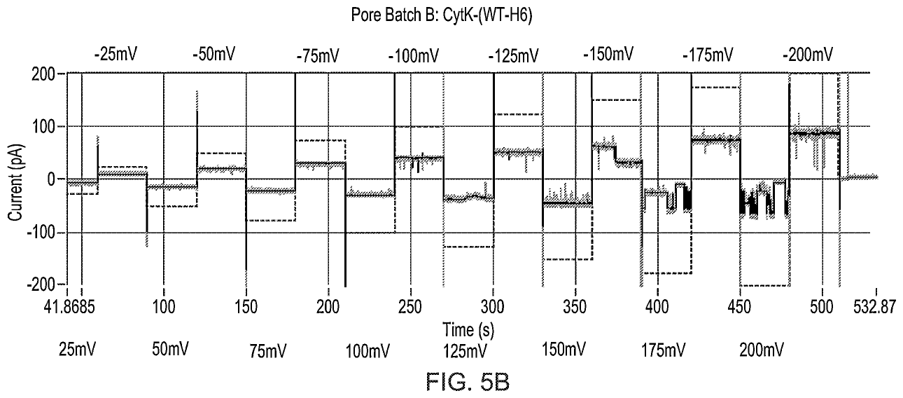

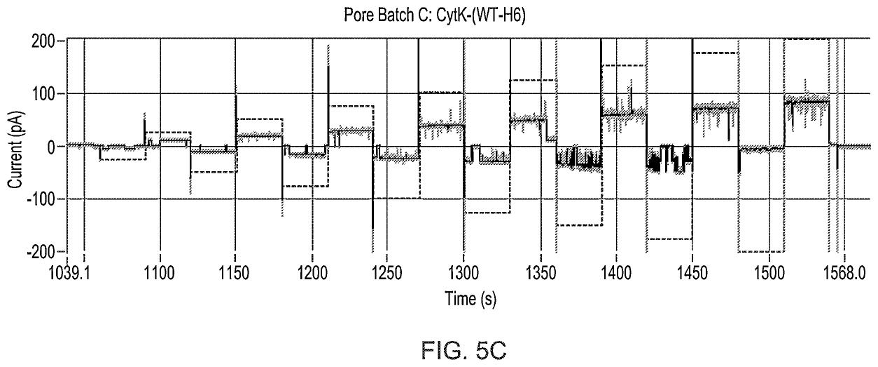

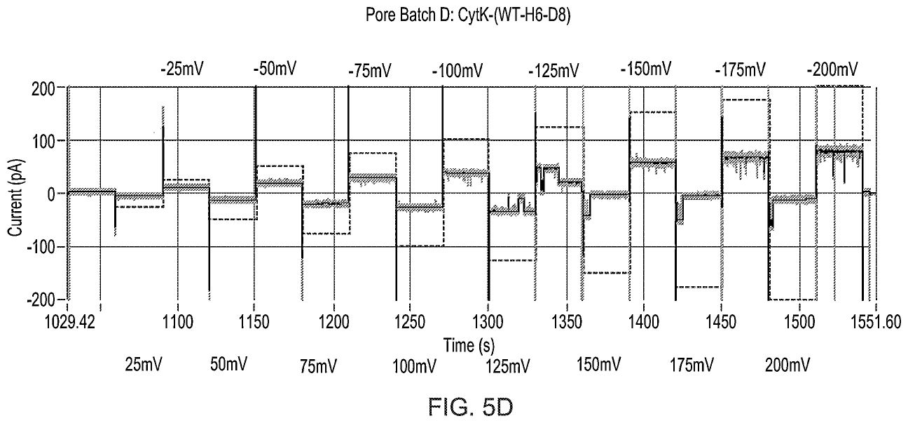

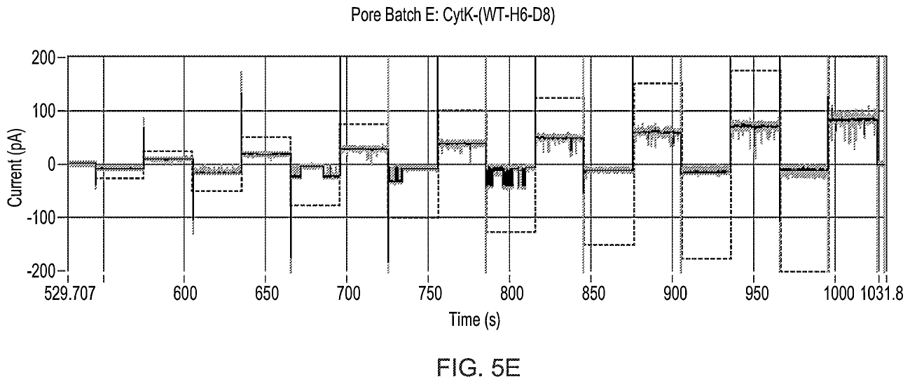

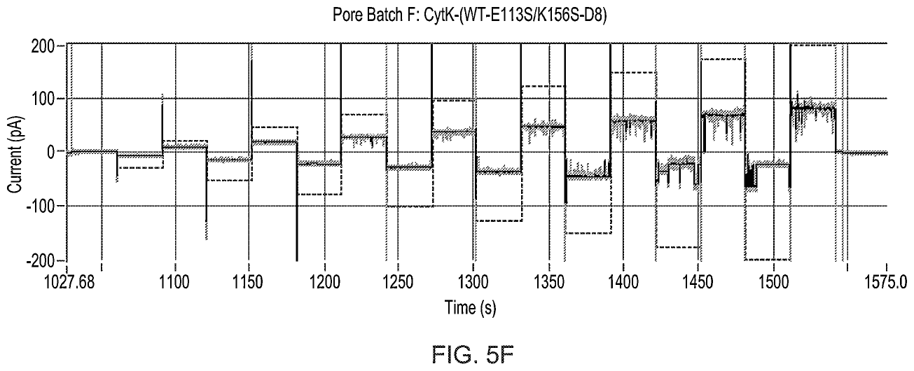

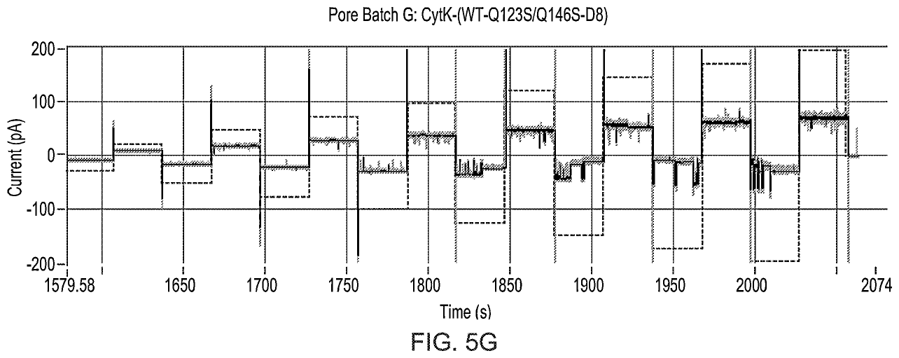

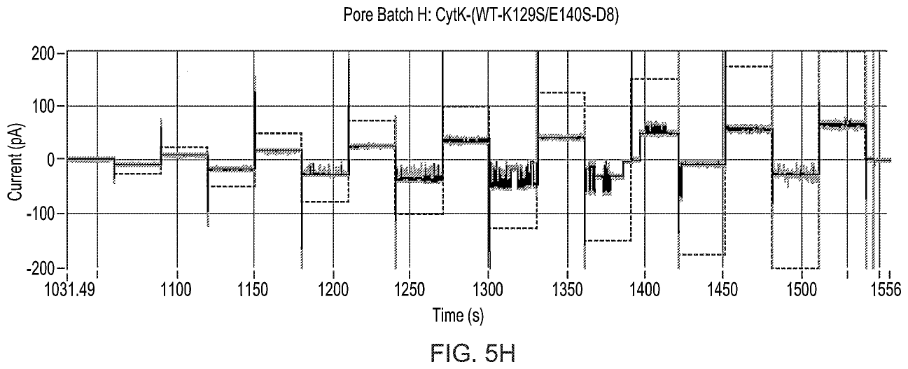

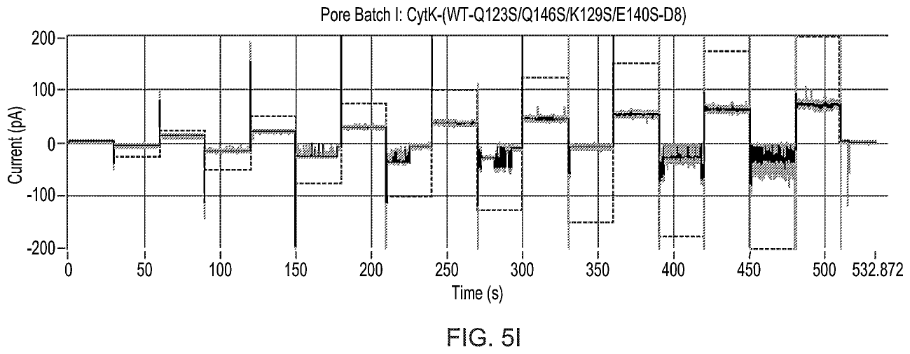

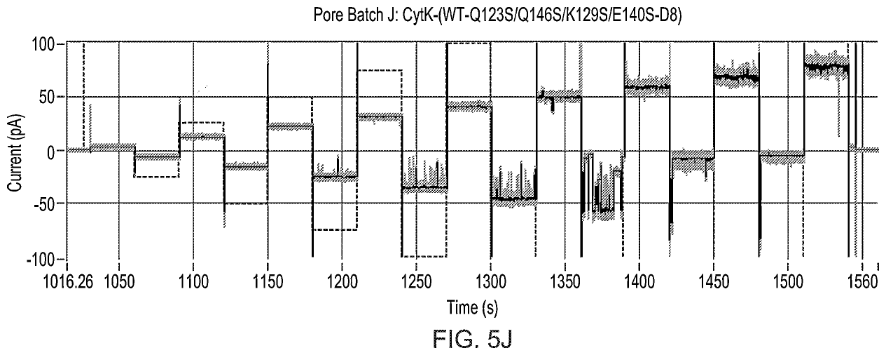

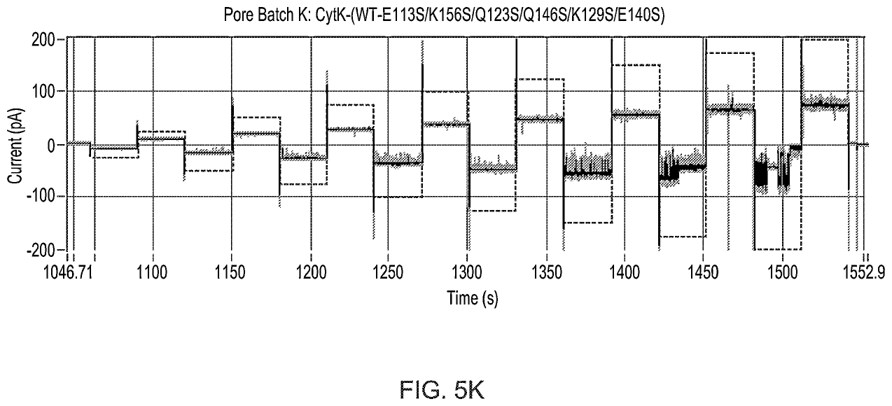

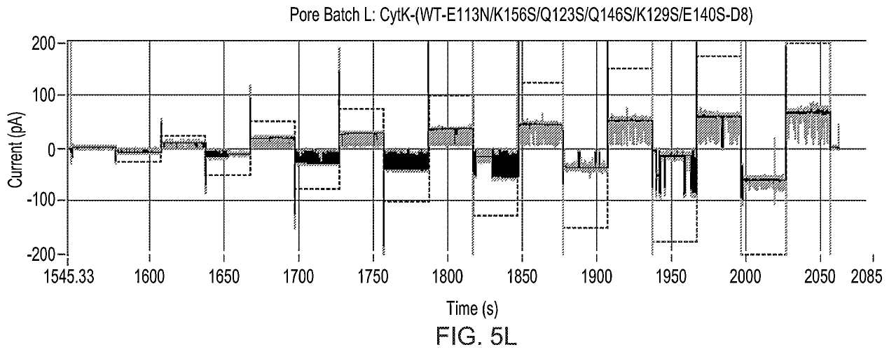

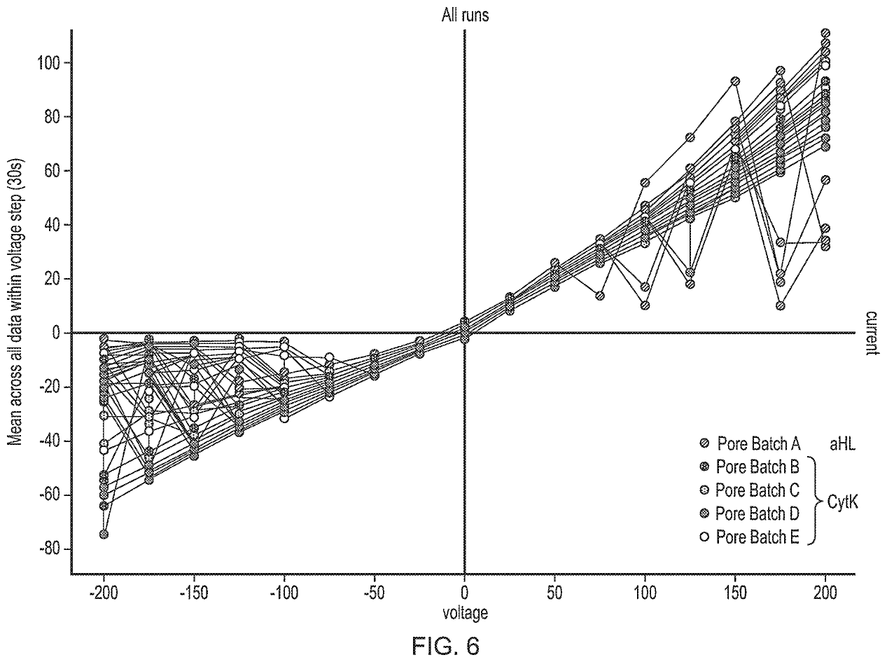

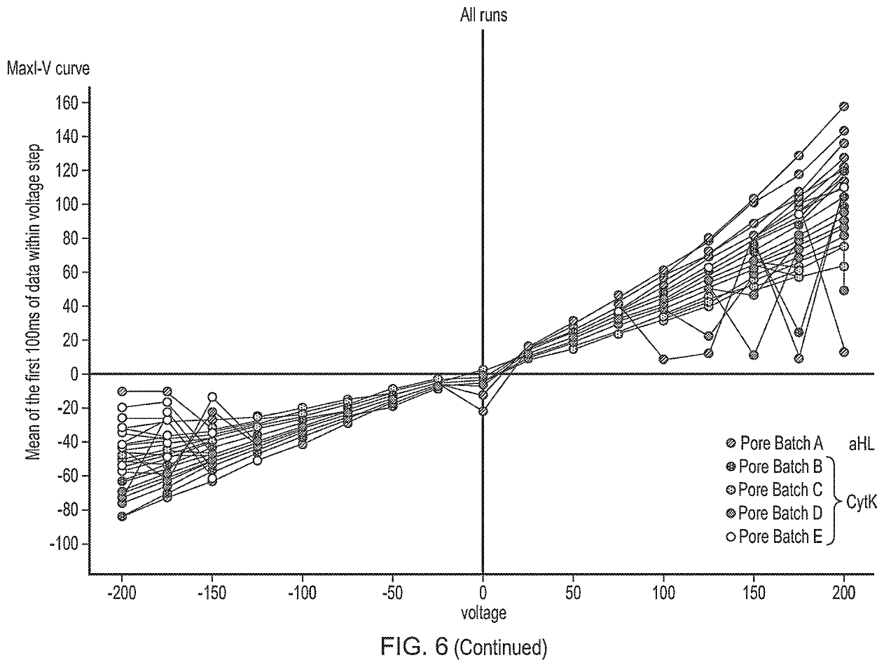

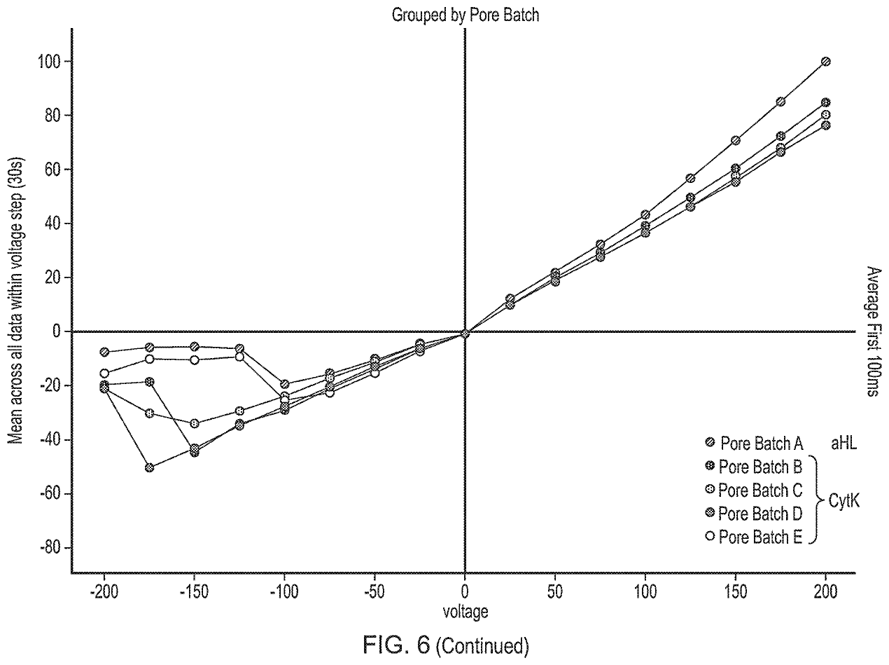

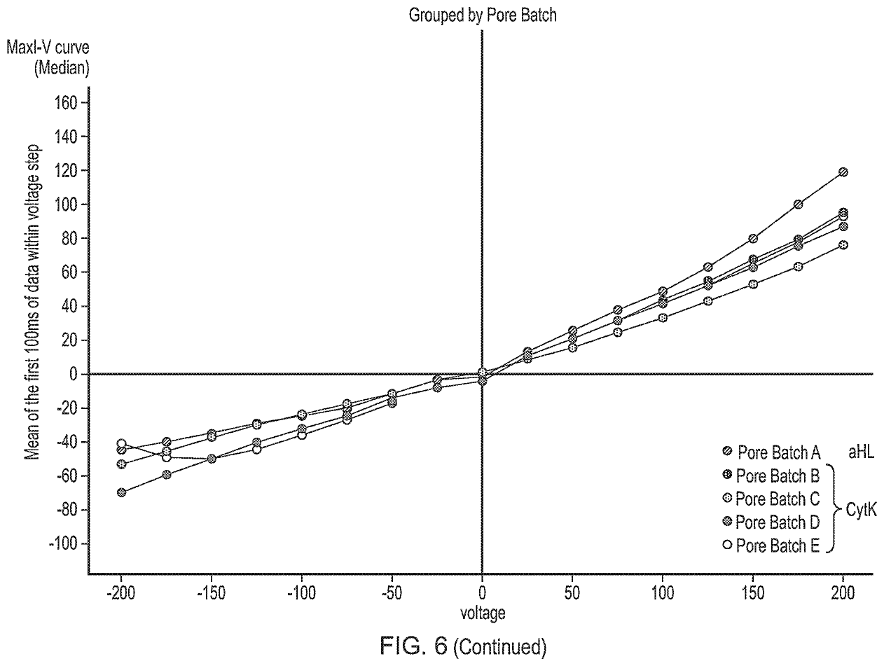

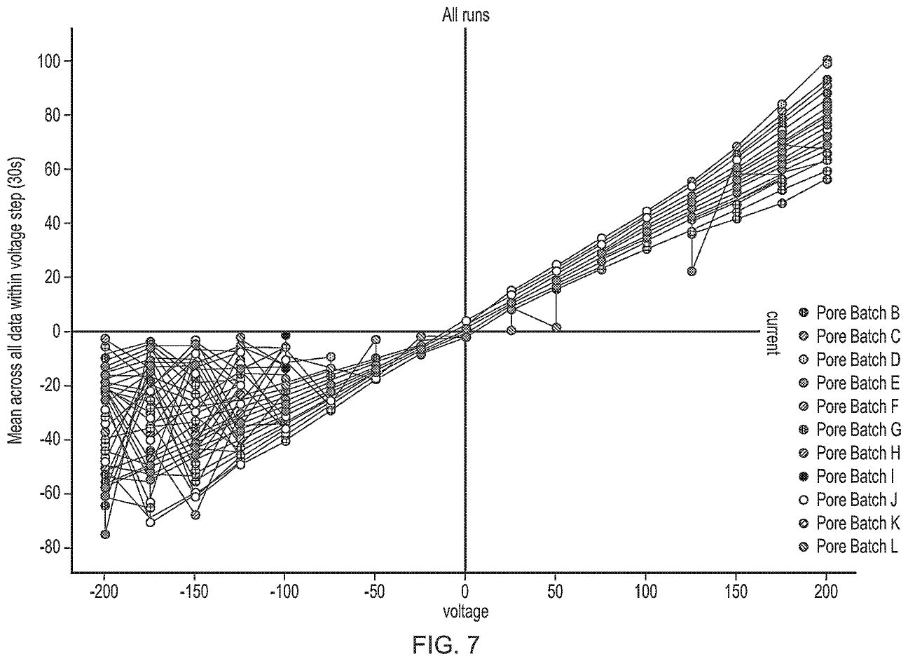

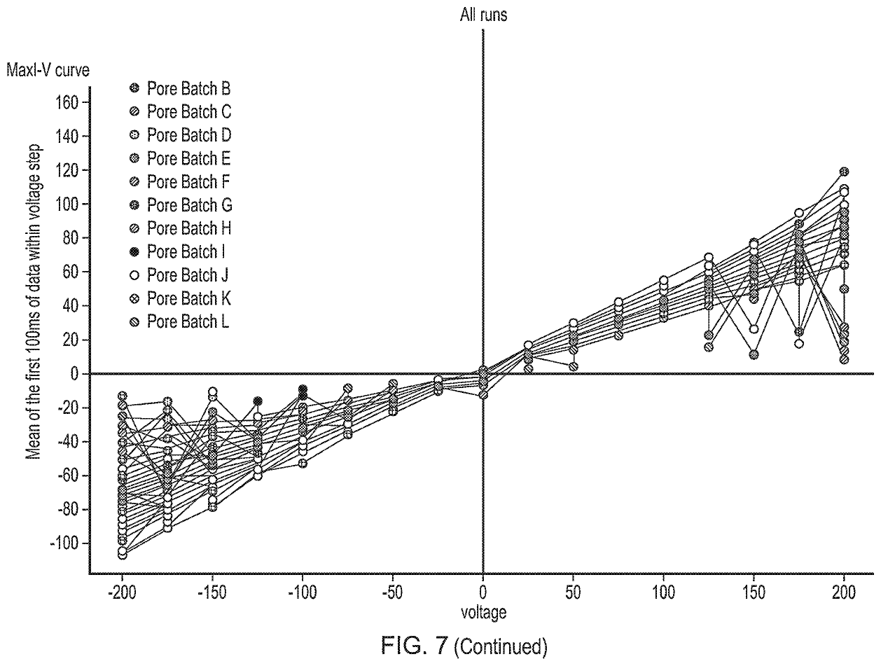

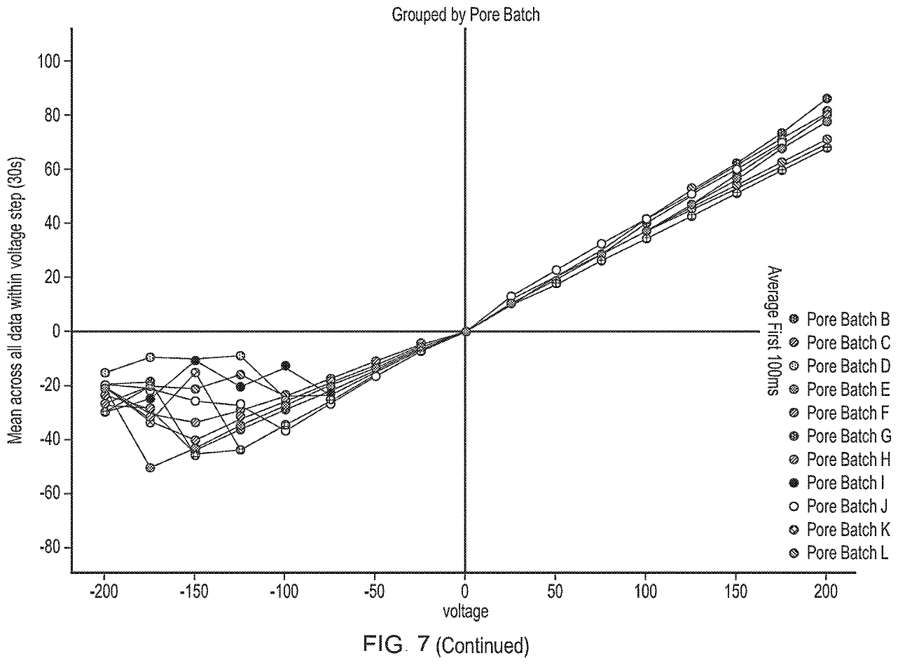

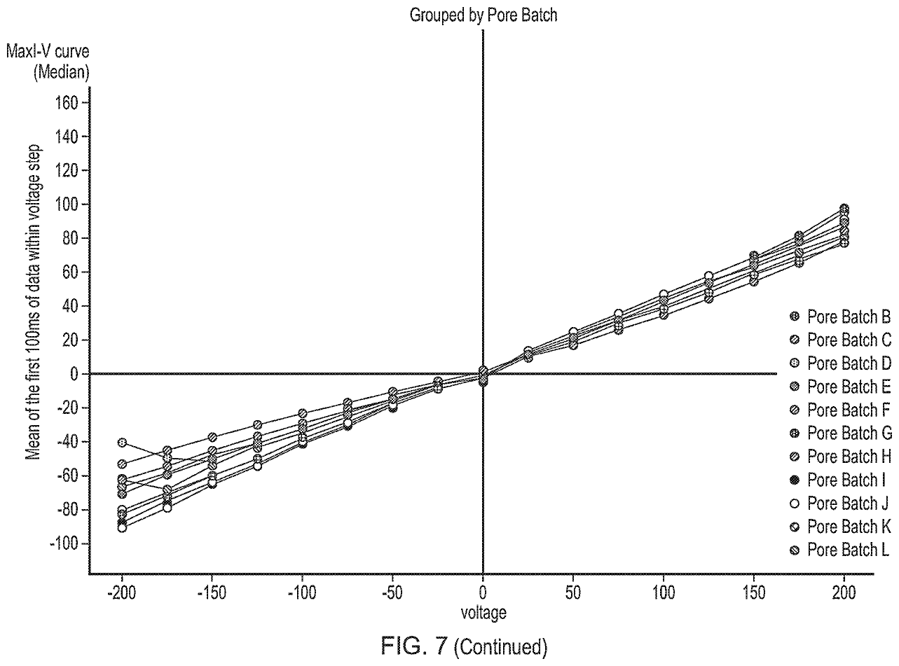

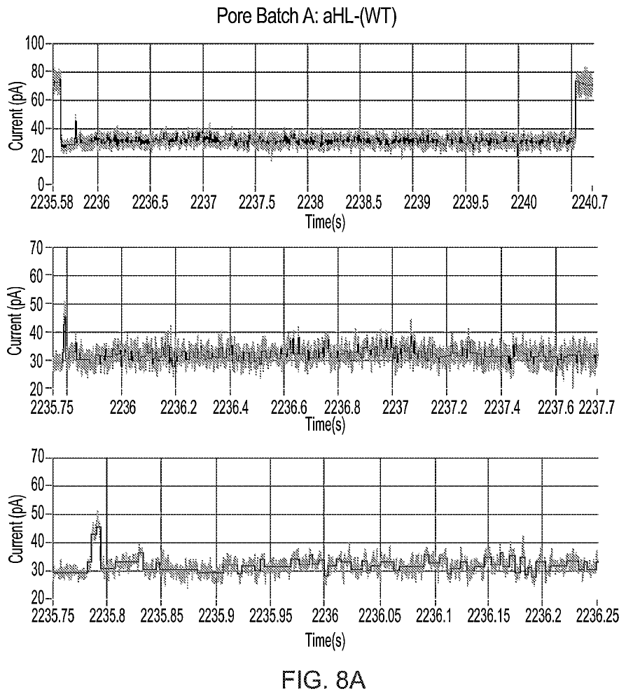

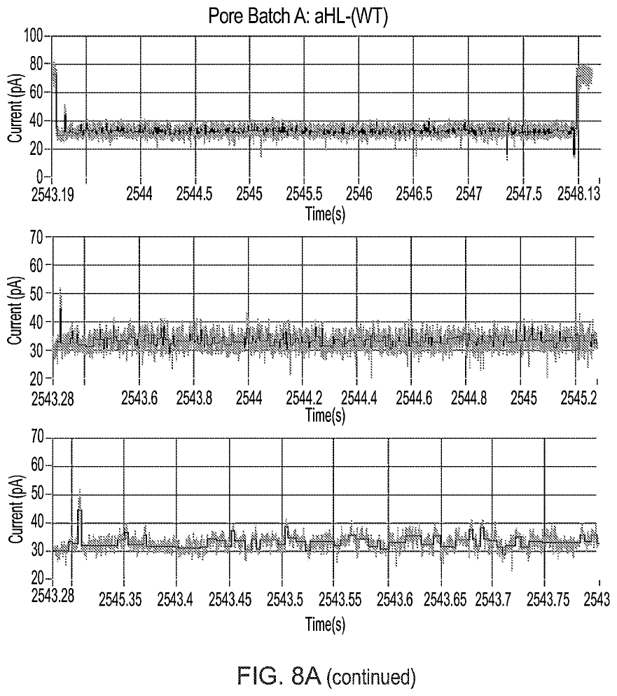

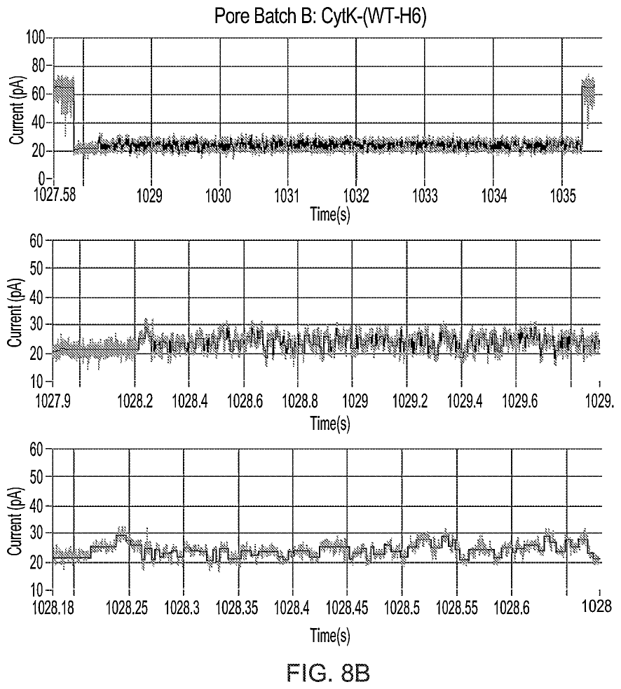

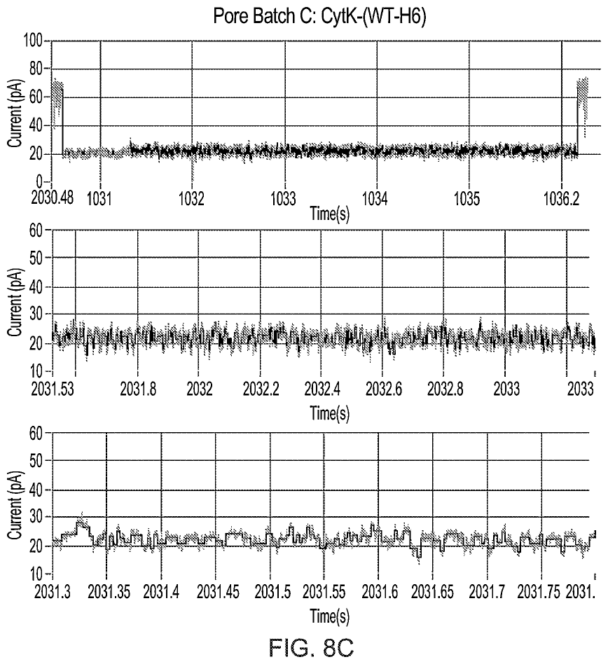

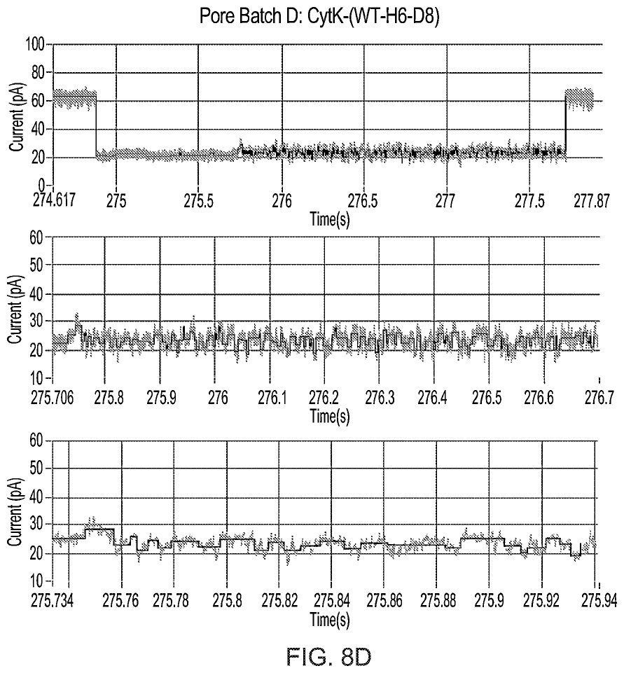

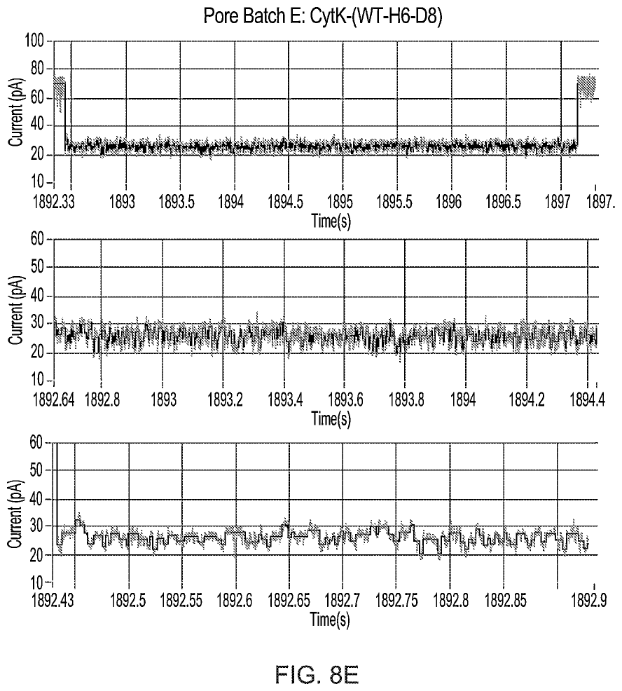

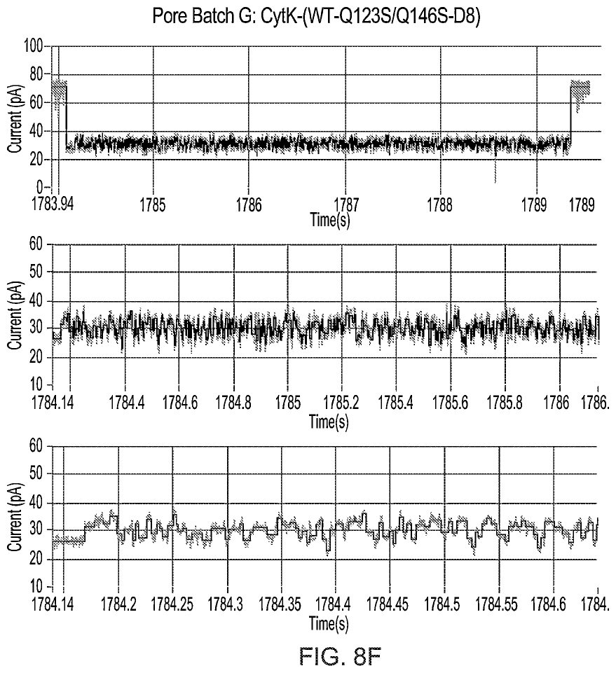

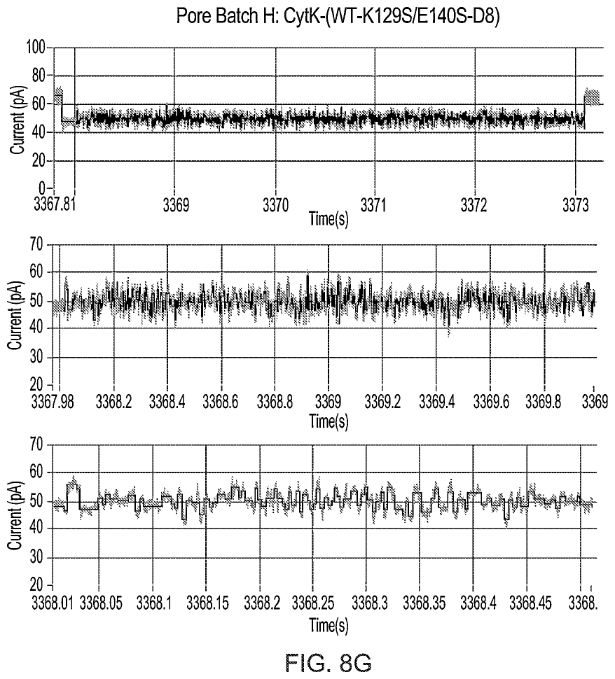

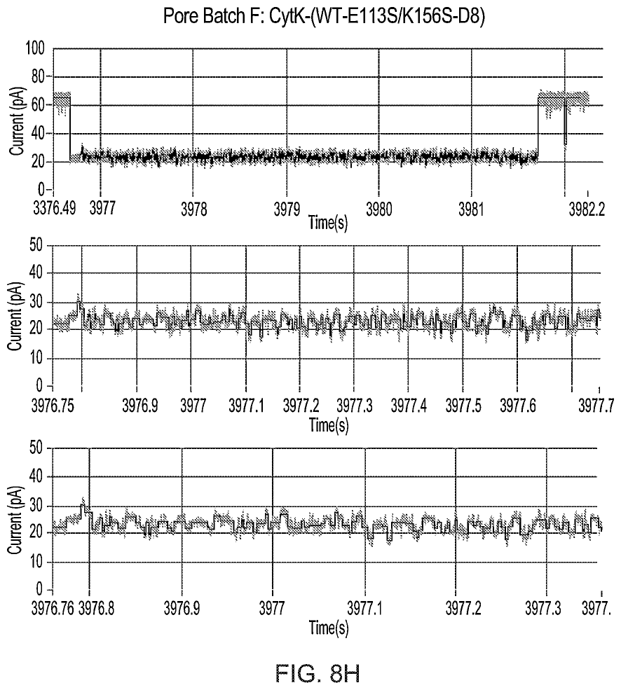

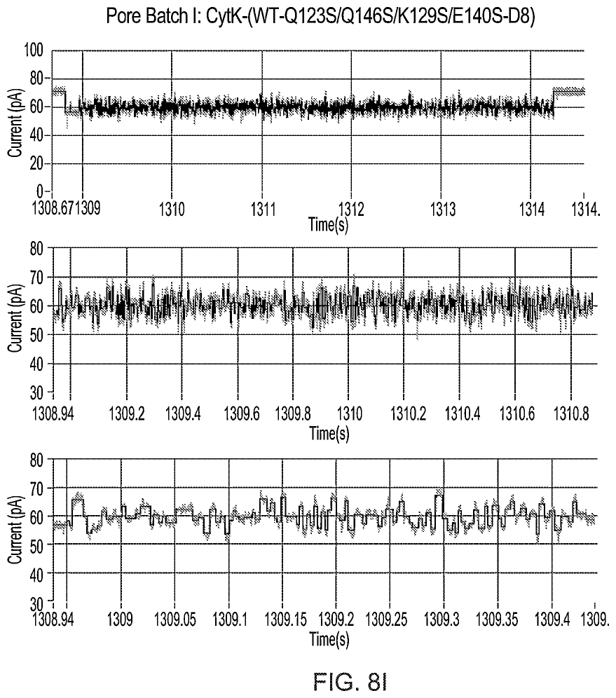

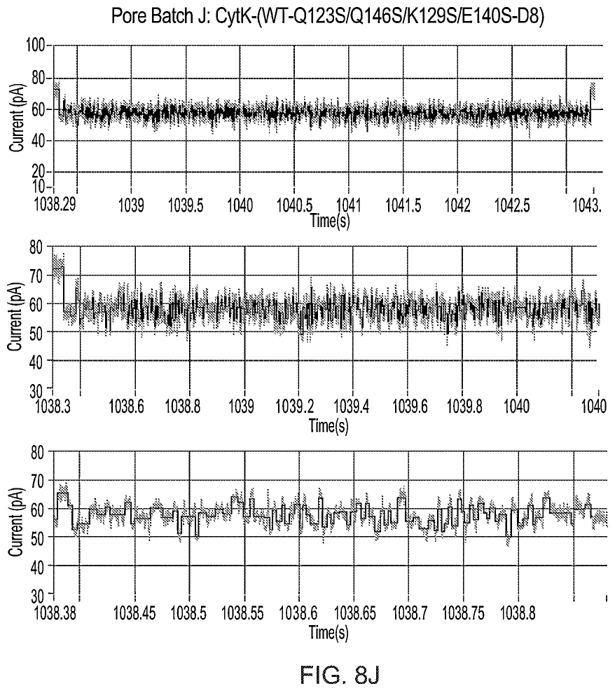

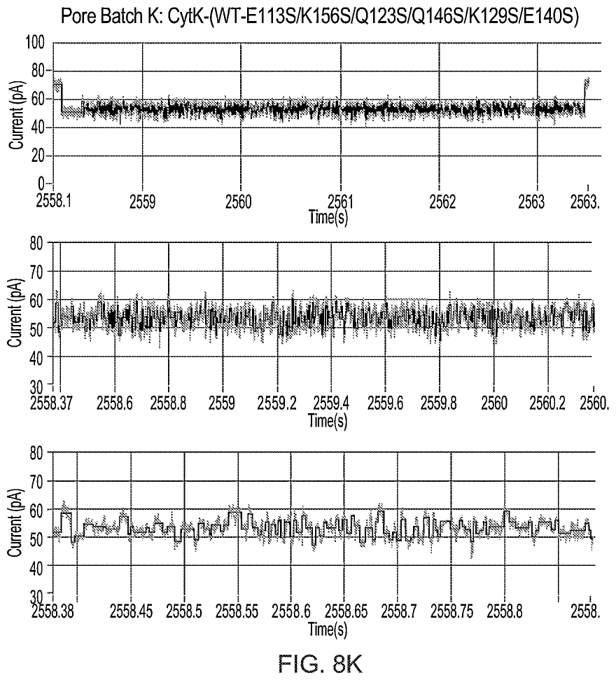

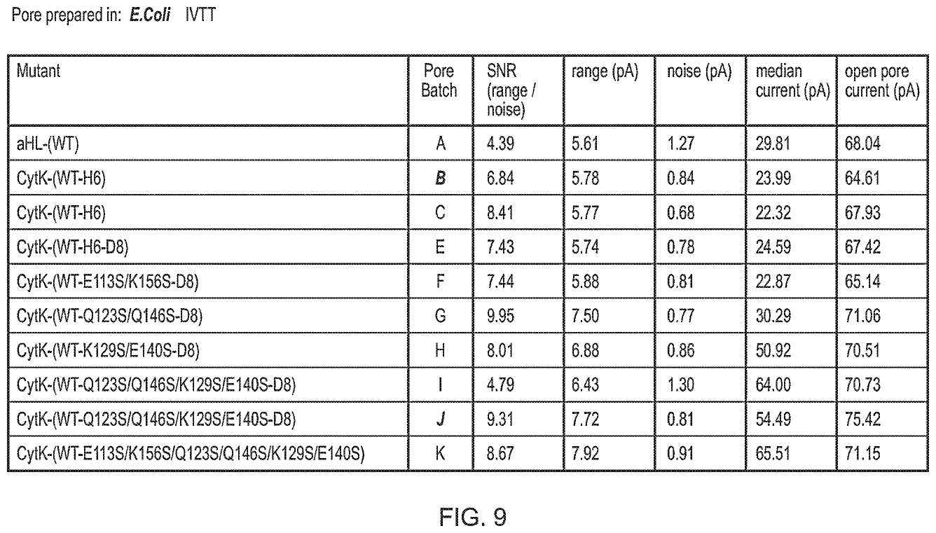

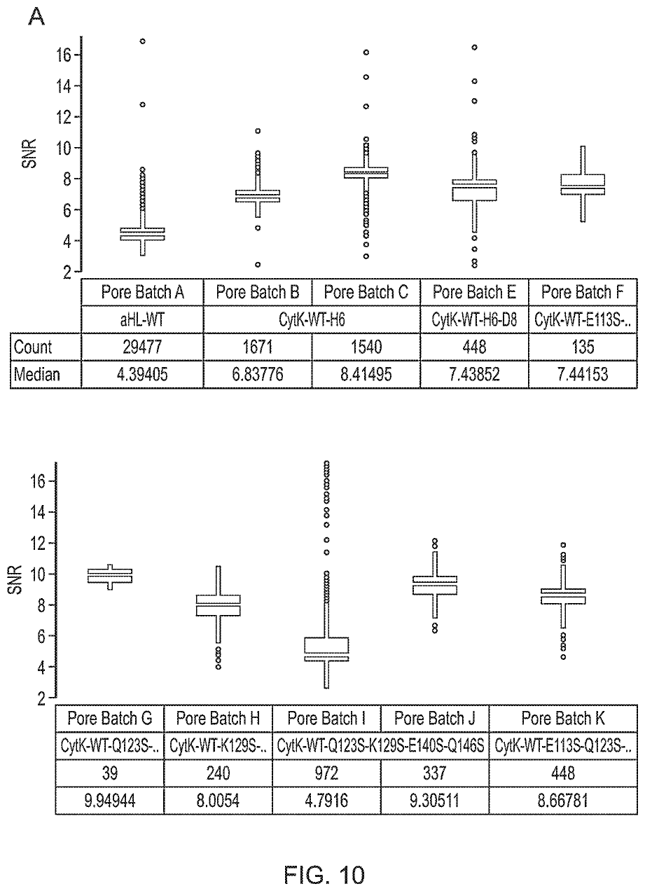

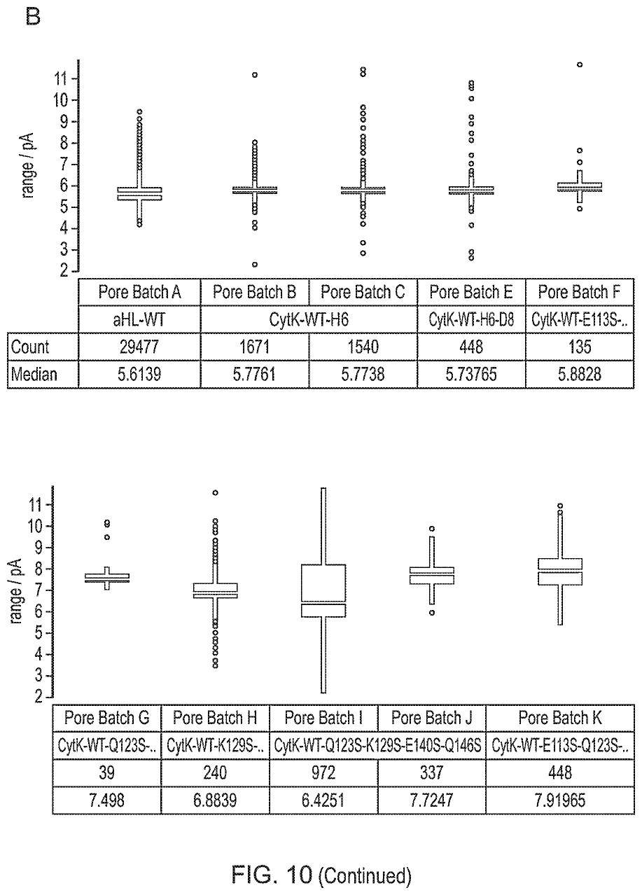

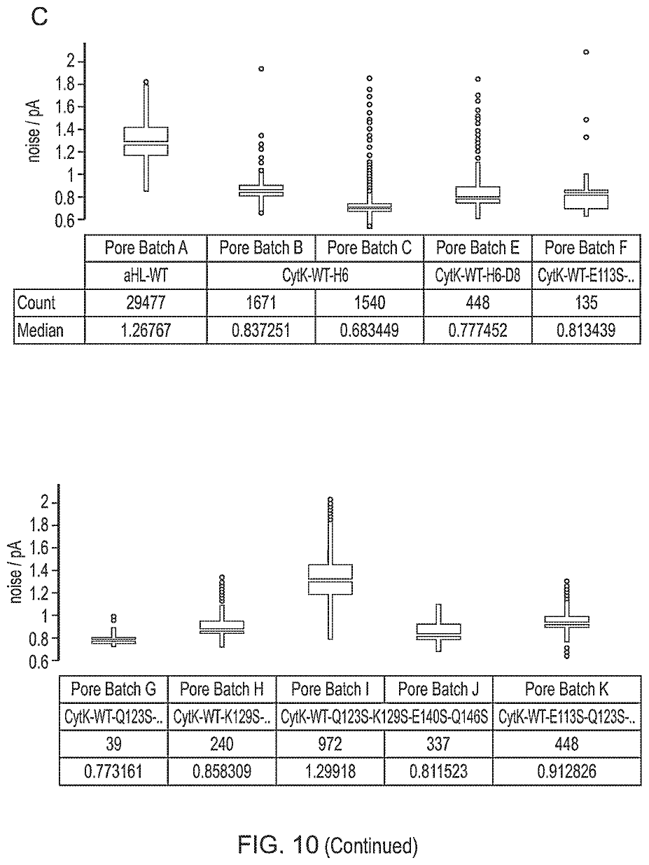

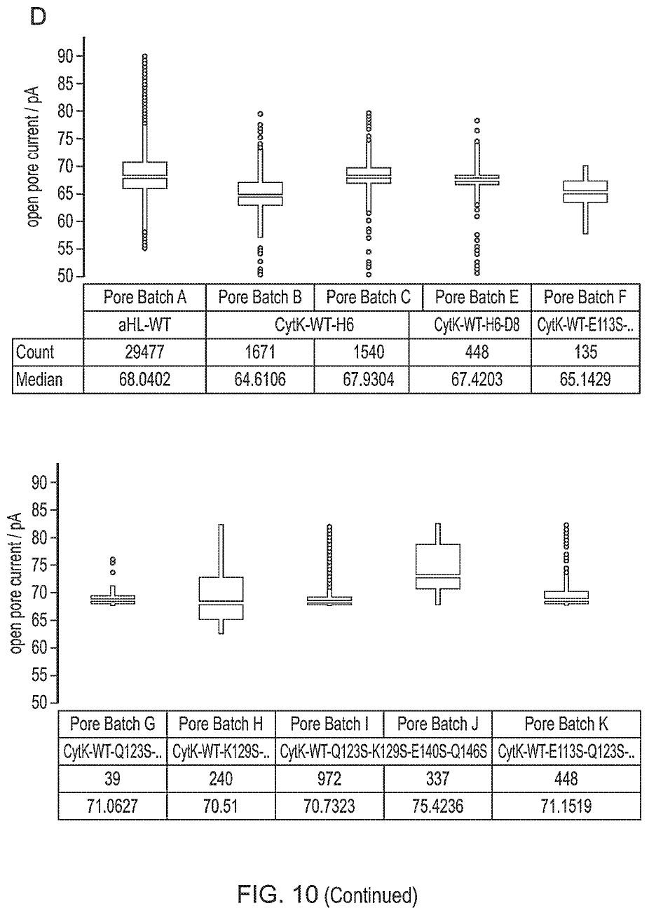

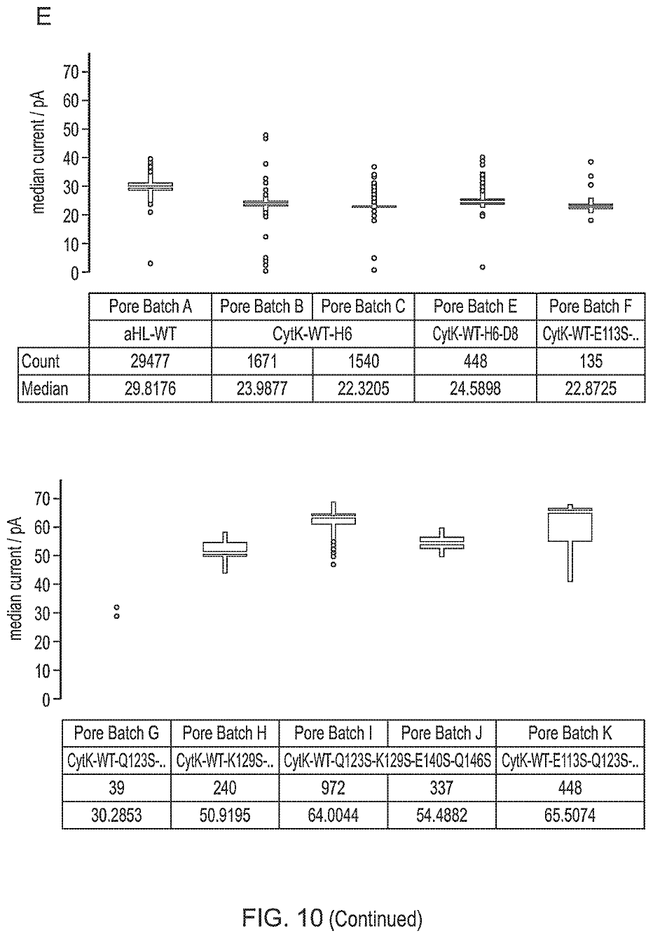

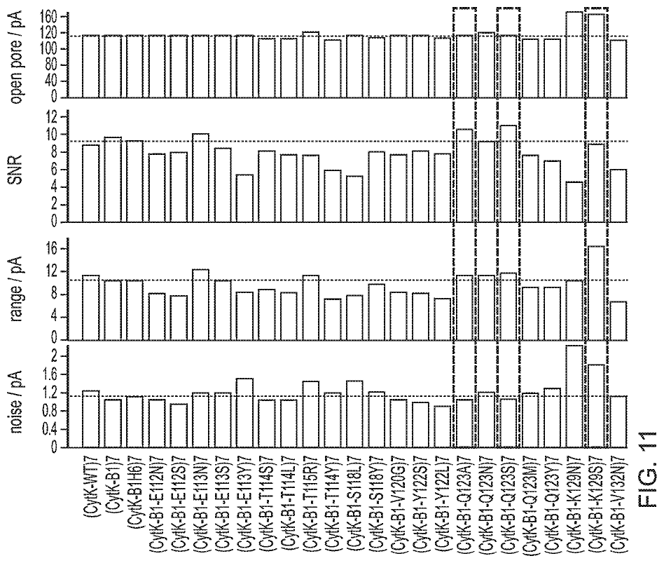

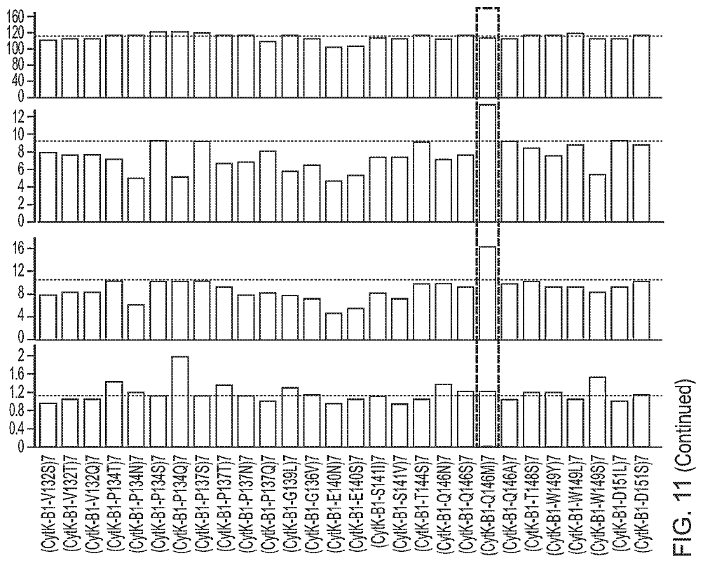

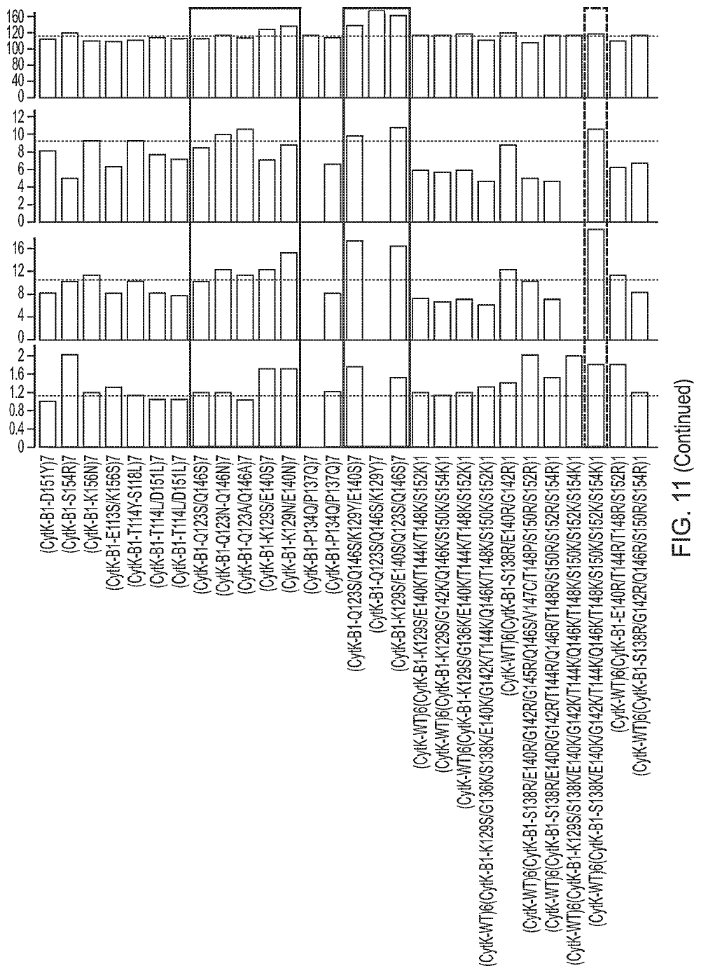

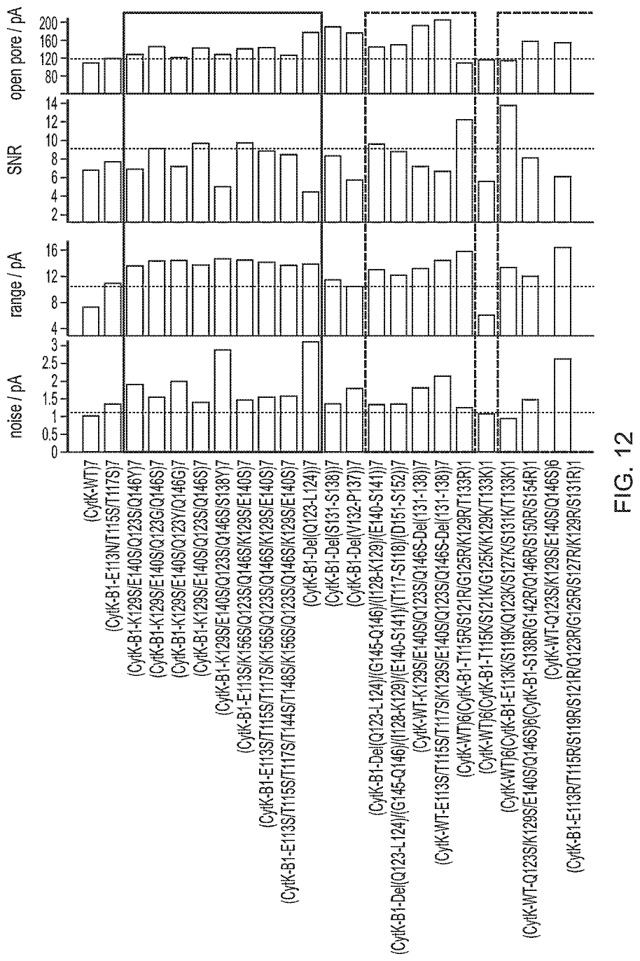

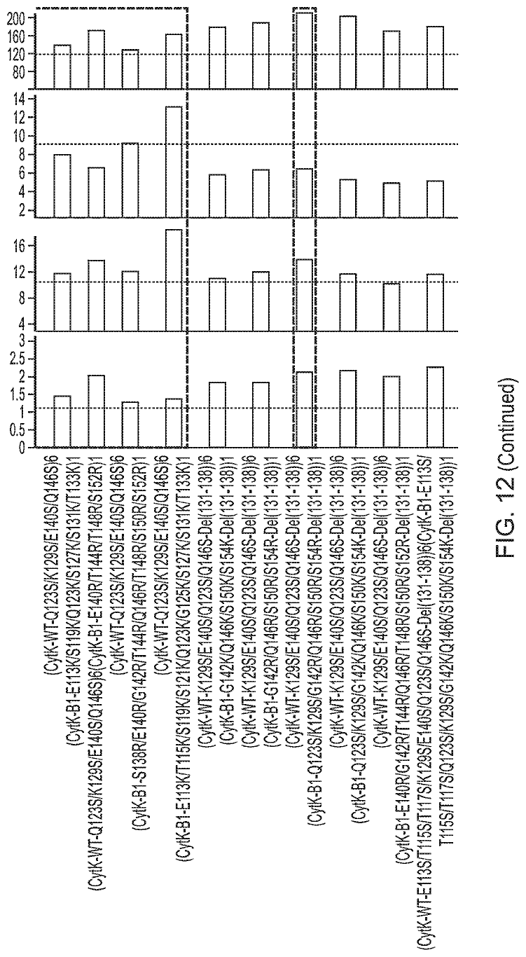

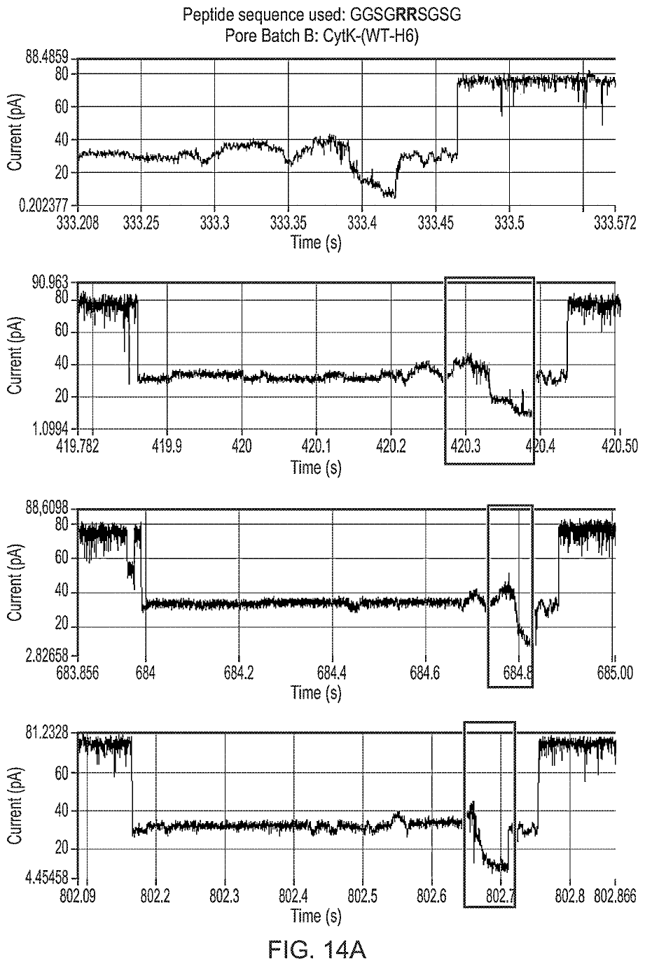

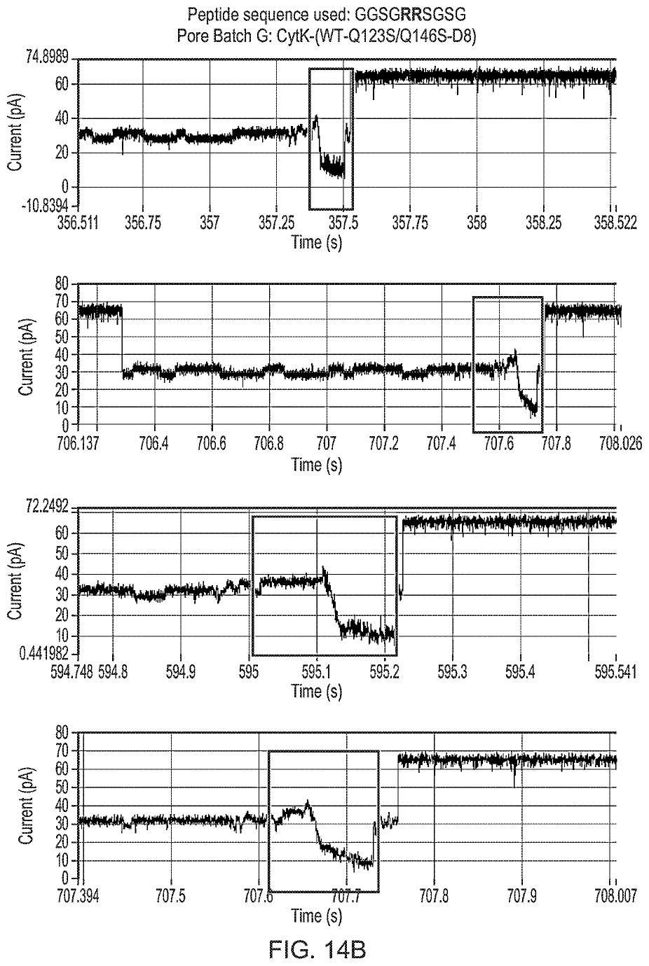

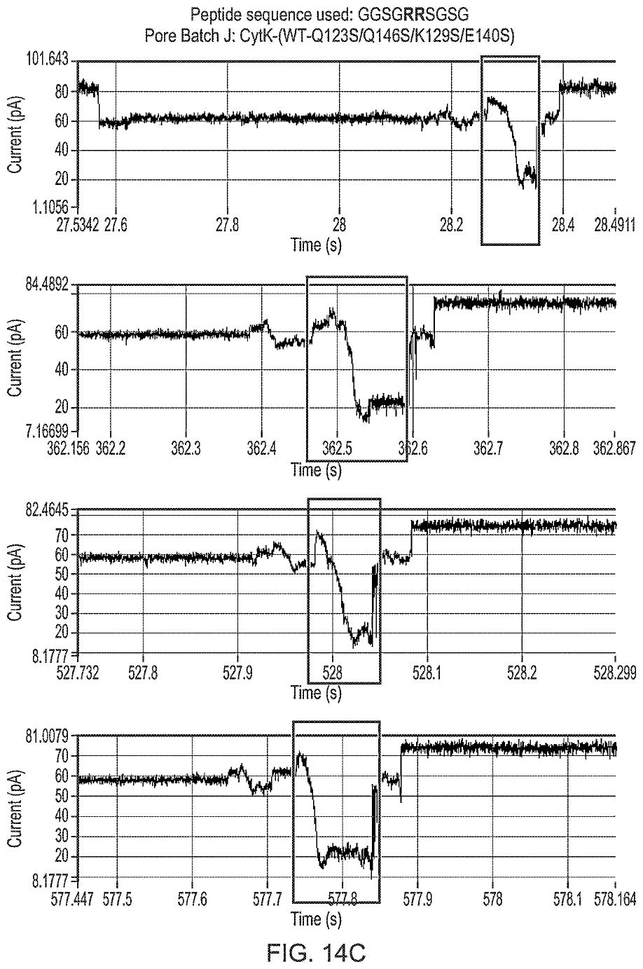

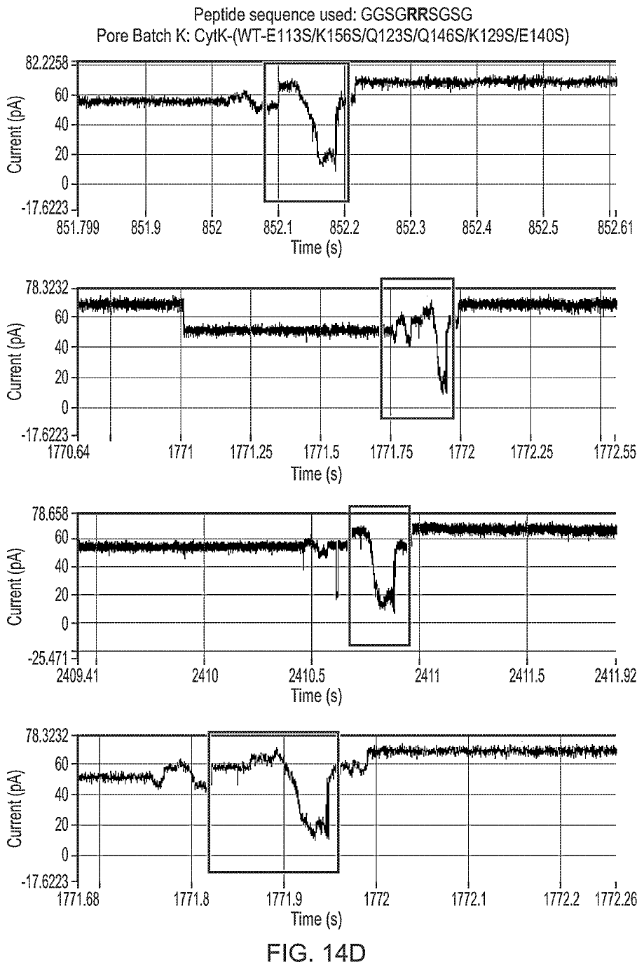

See particularly FIGS. 5-16 and their legends.

DNA encoding the mature form of the CytK protein was synthesized by GenScript USA Inc. and cloned into a pT7 vector containing ampicillin resistance gene. DNA concentration was adjusted to 400 ng/μL.

The plasmid DNA was thawed at room temperature and mixed by slowly pipetting up and down. Chemically competent BL21 (DE3) E. coli cells were thawed on ice. 1 μl of DNA at 400 ng/μl was added to the cells and mixed by slowly pipetting up and down. This was then left on ice for 25 minutes before heat shocking the cells at 42° C. for 45 seconds. The cells were then left on ice for 2 minutes. 250 μl of SOC (Sigma, S1797) media that was pre-warmed to 37° C. was added to the cells and left for one hour at 37° C. with shaking. Half the cells were then plated out on a big LB agar plate containing 50 μg/ml ampicillin and then left to incubate overnight at 37° C.

A single colony of the transformed BL21 (DE3) cells were inoculated in 100 ml LB medium with 100 μg/ml carbenicillin. This starter culture was incubated overnight at 37° C. and 250 rpm in a 500 ml flask. A 500 ml LB medium containing 100 μg/ml carbenicillin was added to a 2.5 l flask. This was then added to 5 ml of starter culture (dilution 1:100) and the cells were left to divide at 37° C. and 250 rpm until O.D 0.6 was reached. Upon reaching O.D. 0.6, the temperature of the incubator was reduced to 18° C. and the cells were induced with 0.2 mM IPTG (final concentration in the medium). The cells were incubated overnight at 18° C. and 250 rpm. Finally, the cells were harvested by spinning them at 6000 g for 30 min at 4° C.

The cell paste was weighed to calculate the right volume of functional lysis buffer to prepare (cells are to be resuspended in 100 ml lysis buffer per 10 g of paste). The required amount of functional lysis buffer was prepared by adding benzonase (10 μl/100 ml) and 4 tablets of protease inhibitor cocktail without EDTA to buffer containing 50 mM Tris/HCl, 0.5 M NaCl, pH 8.0 at room temperature. The cells were resuspended in functional lysis buffer and mixed for 1 hour at room temperature with a magnetic stirrer. The cell suspension was frozen at −80° C. and allowed to thaw at room temperature. DDM was added to the cell suspension at a final concentration of 1% and mixed again for 1 hour at 37° C. with a magnetic stirrer. The cell extract was transferred to 40 ml Beckman tubes and spun at 50,000 g rpm for 30 minutes at room temperature. The supernatant was then filtered through a 0.22 μM PES syringe filter.

Next, the supernatant was loaded onto a 2× 5 mL His Trap FF column (Fisher, 10571680). The column was washed with 50 mM Tris, 0.5 M NaCl, 5 mM imidazole, 0.1% DDM, pH 8.0 (mobile phase A) until a stable baseline of 10 column volumes (CV) was maintained. The column was then washed with 50 mM Tris, 2 M NaCl, 5 mM imidazole, 0.1% DDM, pH 8.0 before being returned to the 150 mM buffer. Elution was carried out with 0.5 M imidazole over a gradient of 0-100% over 20CV, where mobile phase B comprised 50 mM Tris, 0.5 M NaCl, 0.5 M imidazole, 0.1% DDM, pH 8.0.

The fractions of interest from the HisTrap purification were identified via SDS-PAGE. The peak was pooled and then concentrated down using a 50 kDa MWCO (Millipore, UFC905024) to approximately 1 ml. The concentrated retained supernatant was subjected to gel filtration on a 320 ml Superdex200 (Fisher, 11390342) in 50 mM Tris, 0.25 M NaCl, 0.1% DDM, pH 8.0. Fractions identified as containing CytK were collected and pooled. Following this, the pooled supernatant was diluted 5× with 50 mM Tris/HCl, 0.1% DDM, pH 9.0. This was then loaded onto a POROS HQ10 column pre-equilibrated in 50 mM Tris/HCl, 0.1% DDM, pH 9.0. The column was washed with 50 mM Tris/HCl, 0.1% DDM, pH 9.0 until a stable baseline over 10CV was achieved before starting the gradient. A gradient from 50 mM Tris/HCl, 0.1% DDM, pH9.0 to 100% 50 mM Tris/HCl, 0.1% DDM, 1 M NaCl, pH 9.0 was reached over 25CV. The fractions of interest from the POROS HQ10 purification were identified via SDS-PAGE, collected and then assayed in electrophysiology recordings.

### In Vitro Transcription Translation (IVTT) Pore Production

See particularly FIGS. 5-16 and their legends.

For a single 25 μL reaction the following was prepared:

| | | Part no. (Promega part |
|---|---|---|
| | | numbers unless otherwise |
| Component | Volume (μL) | stated) |
| S30 Premix without AA | 10.25 | L215A |
| AA-Cysteine | 1.25 | L447A |
| AA-Methionine | 1.25 | L9968 |
| T7 S30 extract for circular | 7.5 | L414A |
| DNA | | |
| S35 radiolabeled methionine | 0.25 | Perkin Elmer, NEG009A |
| | | 005MC |
| Rifampicin (50 μg/μL) | 0.5 | R8883 |
| DNA (400 ng/μL) | 4 | N/A |

The components above were mixed and incubated at 30° C. and 700 rpm on a Thermo Shaker. Samples were then spun down at 21,000 g for 10 minutes at room temperature. Supernatant was carefully removed and discarded while the pellet was resuspended in 1× Laemmli buffer (BioRad, 1610737) by pipetting up and down. The resuspension was then loaded onto a 7.5% Tris-HCl, pH8.0 slab gel and electrophoresis at 55 V was performed overnight (16 hours) in 1×TGS running buffer (Sigma, T7777). The gel was then dried under vacuum for 5 hours at 50° C. An X-ray film (Sigma, Z370371) was exposed to the gel for 2 hours and developed using a combination of Devalex (Champion, 120102) and Fixaplus (Champion, 120202X) solution in an X-ray film developer. The film was then placed over the dried gel and the relevant bands were extracted, using the film as reference. Each extracted band was rehydrated in 100 μL of 50 mM Tris/HCl, 2 mM EDTA, pH 8.0 buffer and crushed with a pestle until a homogenous slurry was obtained. The slurry was incubated overnight at room temperature, added to a 0.45 μm CoStar column (Sigma, CLS8162) and spun at 21,000 g for 10 minutes. The supernatant was collected and assayed in electrophysiology recordings.

### IV Curves and DNA Squiggle

See particularly Figure legends 5-12 and their legends.

Electrical measurements were acquired from aHL and CytK wild-type and mutant nanopores that were inserted into MinION flow cells. After a single pore inserted into the block co-polymer membrane, 2 mL of a buffer comprising 25 mM Potassium Phosphate, 150 mM Potassium Ferrocyanide (II), 150 mM Potassium Ferricyanide (III), pH 8.0 was flowed through the system to remove any excess CytK nanopores. The ionic current profiles through the nanopores were then obtained as the voltage was gradually increased in 25 mV steps every 30 seconds in both the negative and positive direction from (−)25 mV up to (−)200 mV.

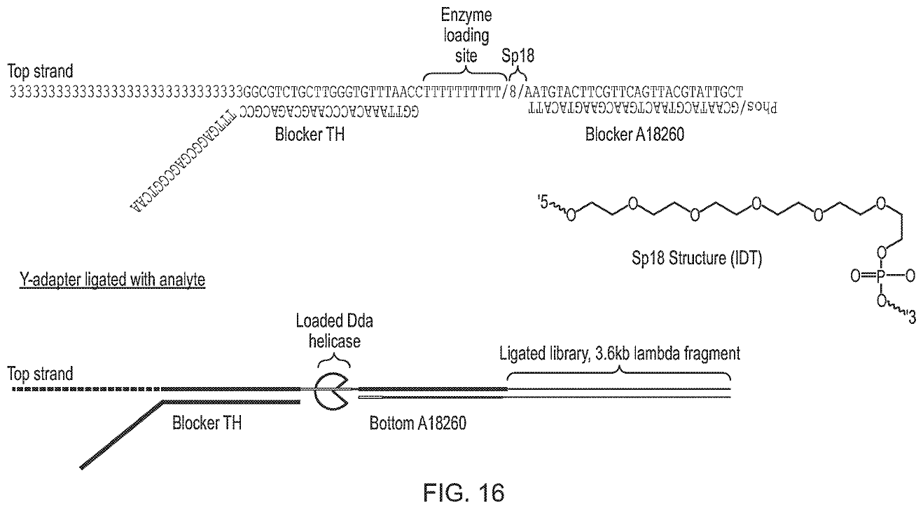

A Y-adapter was prepared by annealing DNA oligonucleotides shown in FIG. 16. A DNA motor (Dda helicase) was loaded and closed on the adapter. The subsequent material was HPLC purified. The Y-adapter contains a 30 C3 leader section for easier capture by the nanopore and a side arm for tethering to the membrane.

The analyte being used to assess the DNA squiggle was a 3.6-kilobase ssDNA section from the 3′ end of the lambda genome. Preparation of the analyte, ligating the analyte to the Y-adapter, SPRI-bead clean-up of the ligated analyte and addition to a MinION flow cell was carried out using the Oxford Nanopore Technologies Q-SQK-LSK109 protocol.

Electrical measurements were acquired using MinION Mk1b from Oxford Nanopore Technologies. A standard sequencing script at −180 mV was run for 1-6 hours, with static flicks every 5 minute to remove extended nanopore blocks. Raw data was collected in a bulk FAST5 file using MinKNOW software (Oxford Nanopore Technologies).

### Peptide Squiggles

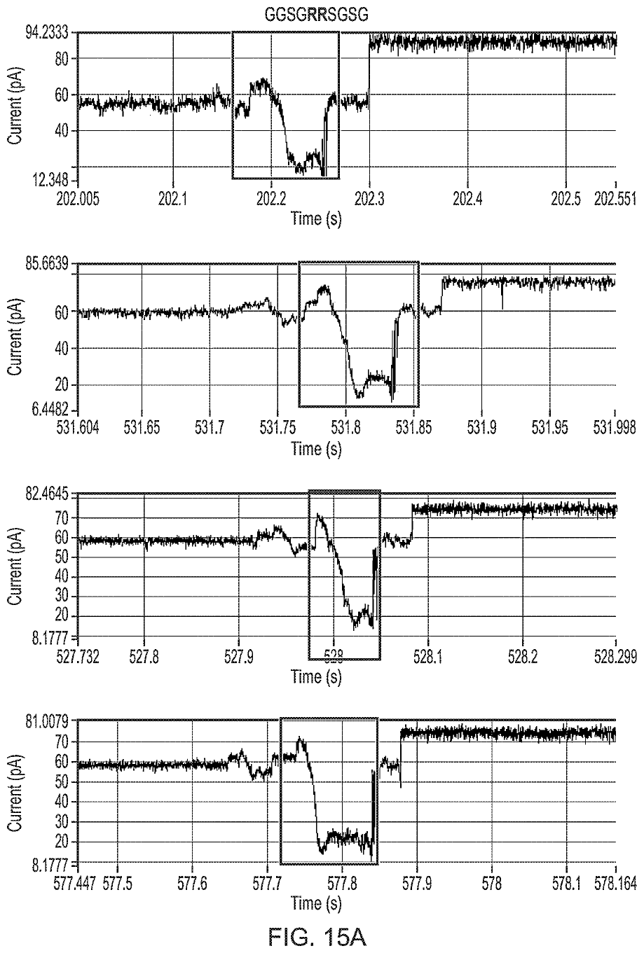

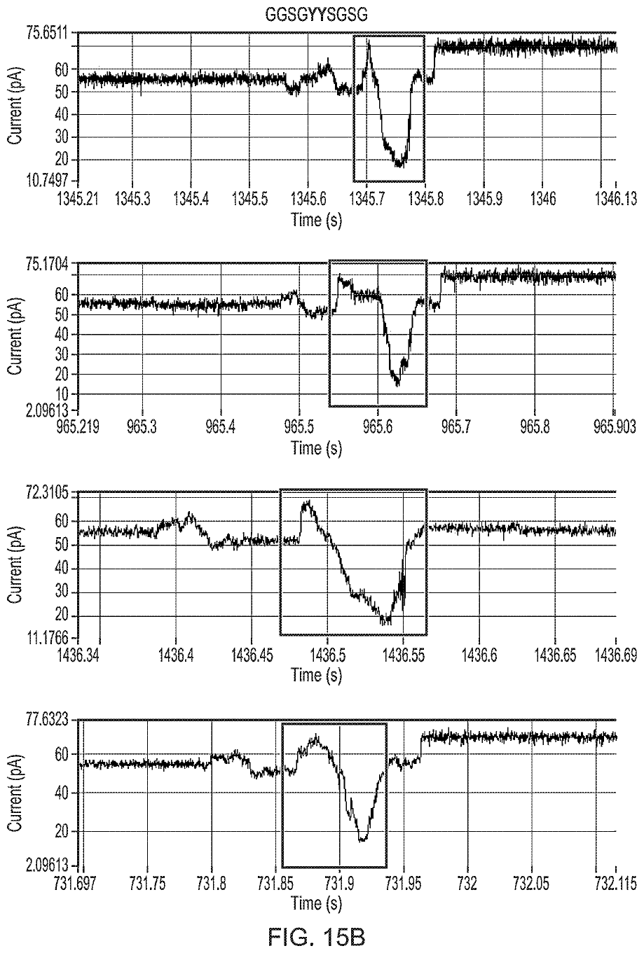

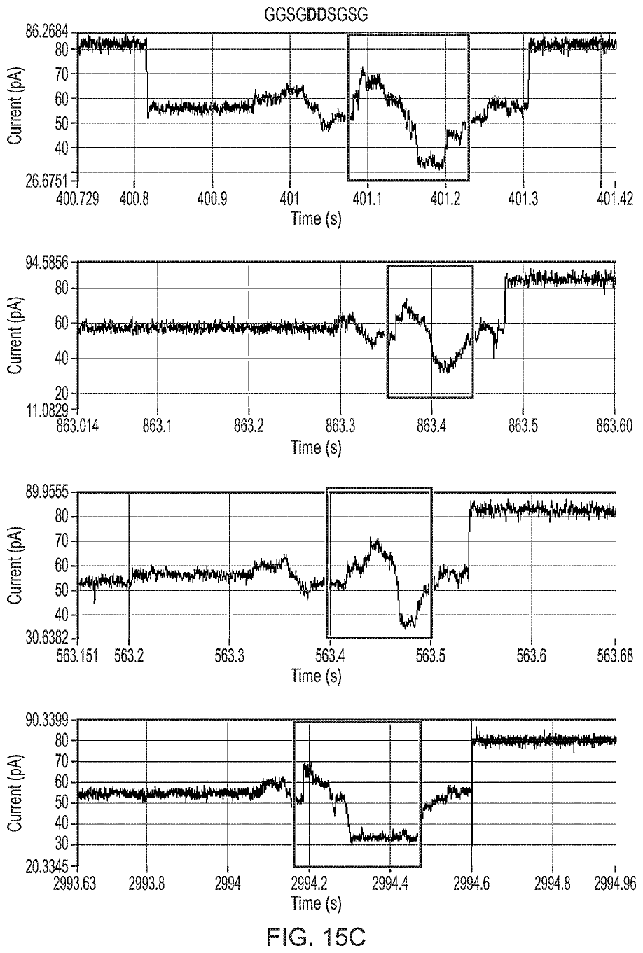

See particularly FIGS. 15 and 16, and their legends.

Example current versus time traces as a peptide translocates through CytK wild-type and mutants were obtained by using a conjugate comprising a polypeptide flanked by two pieces of polynucleotide; a dsDNA Y adapter (DNA1) and a dsDNA tail (DNA2). A polynucleotide-handling protein at the cis side of the nanopore controls the movement of the conjugate by first unwinding DNA1 and translocating 5′-3′ on ssDNA, then sliding across the polypeptide section to finally unwind the DNA2 segment. As this construct moves from the cis to trans side of the nanopore, the DNA and polypeptide sections can be visualized on a current vs time plot.

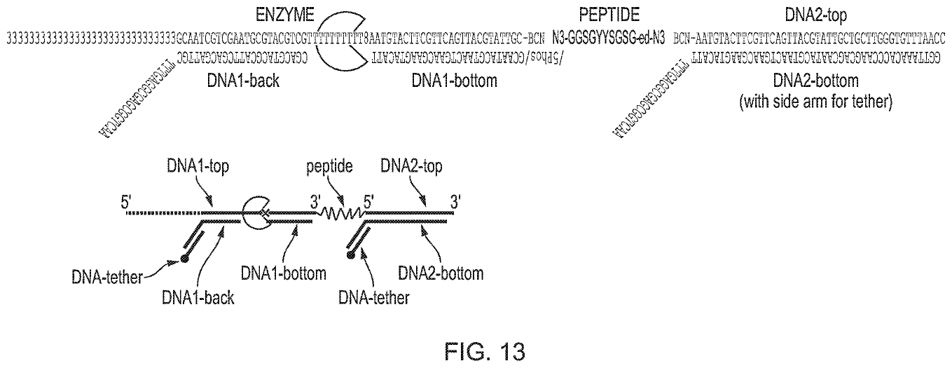

A Y-adapter was prepared by annealing DNA oligonucleotides (FIG. 13). A DNA motor (Dda helicase) was loaded and closed on the adapter. The subsequent material was HPLC purified. The Y adapter contains a 30 C3 leader section for easier capture by the nanopore and a side arm for tethering to the membrane. The DNA tail was made by annealing two DNA oligonucleotides, it also contains a side arm for tethering resulting in two tethering sites per construct to increase efficiency of capture.

The polypeptide analytes were obtained with azide moieties at the N-terminus and directly after the C-terminus using an ethyl diamine spacer in line with the peptide backbone. Each analyte was then conjugated to the Y-adapter and DNA tail via copper-free Click Chemistry reaction between the azide and BCN (bicyclo[6.1.0]nonyne) moieties. The sample was purified using Agencourt AMPure XP (Beckman Coulter) beads, with two washes in 28% PEG 8K, 2.5M NaCl, 25 mM Tris (pH 8.0) buffer, and eluted into 10 mM Tris-Cl, 50 mM NaCl (pH 8.0).

Electrical measurements were acquired using MinION Mk1b from Oxford Nanopore Technologies and a custom MinION flow cell with either CytK wild-type or CytK mutant pores inserted. Flow cells were flushed with a tether mix containing 50 nM of DNA tether and SQB buffer lacking ATP. Initially 800 μL of tether mix was added for 5 minutes, then a further 200 μL of mix were flowed through the system with the SpotON port open. DNA-peptide constructs were prepared at 0.5 nM concentration in buffer like SQB from Oxford Nanopore Technologies sequencing kit (SQK-LSK109) but lacking ATP, and LB from Oxford Nanopore Technologies sequencing kit (SQK-LSK109), yielding “sequencing mix”. 75 μL of the sequencing mix was added to a MinION flow cell via the SpotON flow cell port. The mixture was incubated on the flow cell for 5-10 minutes to allow for construct tethering and subsequent capture by the nanopores. In the absence of ATP, the DNA motor remains stalled on the spacer region of the Y-adapter, the conjugates are captured by the nanopores but there is no translocation. After the incubation, 200 μL of SQB from Oxford Nanopore Technologies sequencing kit (SQK-LSK109) was added, in the presence of ATP the captured DNA-peptide conjugate is moved across the nanopore by the helicase resulting in a reproducible current footprint.

A standard sequencing script at −180 mV was run for 1-6 hours, with static flicks every 1 minute to remove extended nanopore blocks. Raw data was collected in a bulk FAST5 file using MinKNOW software (Oxford Nanopore Technologies).

### Description of the Sequence Listing

SEQ ID NO: 1 shows the wild type amino acid sequence of a Cytotoxin K monomer.

SEQ ID NO: 2 shows a polynucleotide sequence encoding the wild type Cytotoxin K monomer.

SEQ ID NO: 3 shows the amino acid sequence of exonuclease I enzyme (EcoExo I) from E. coli.

SEQ ID NO: 4 shows the amino acid sequence of the exonuclease III enzyme from E. coli. This enzyme performs distributive digestion of 5′ monophosphate nucleosides from one strand of double stranded DNA (dsDNA) in a 3′-5′ direction. Enzyme initiation on a strand requires a 5′ overhang of approximately 4 nucleotides.

SEQ ID NO: 5 shows the amino acid sequence of the RecJ enzyme from T. thermophilus (TthRecJ-cd). This enzyme performs processive digestion of 5′ monophosphate nucleosides from ssDNA in a 5′-3′ direction. Enzyme initiation on a strand requires at least 4 nucleotides.

SEQ ID NO: 6 shows the amino acid sequence of the bacteriophage lambda exonuclease. The sequence is one of three identical subunits that assemble into a trimer. The enzyme performs highly processive digestion of nucleotides from one strand of dsDNA, in a 5′-3′direction (http://www.neb.com/nebecomm/products/productM0262.asp). Enzyme initiation on a strand preferentially requires a 5′ overhang of approximately 4 nucleotides with a 5′ phosphate.

SEQ ID NO: 7 shows the amino acid sequence of the Phi29 DNA polymerase.

SEQ ID NO: 8 shows the amino acid sequence of Hel308 Mbu.

SEQ ID NO: 9 shows the amino acid sequence of Hel308 Csy.

SEQ ID NO: 10 shows the amino acid sequence of Hel308 Tga.

SEQ ID NO: 11 shows the amino acid sequence of Hel308 Mhu.

SEQ ID NO: 12 shows the amino acid sequence of TraI Eco.

SEQ ID NO: 13 shows the amino acid sequence of XPD Mbu.

SEQ ID NO: 14 shows the amino acid sequence of Dda 1993.

SEQ ID NO: 15 shows the amino acid sequence of Trwc Cba.

SEQ ID NO: 16 shows the polynucleotide sequence encoding the Phi29 DNA polymerase.

SEQUENCE LISTING

SEQ ID NO: 1
MQTTSQVVTDIGQNAKTHTSYNTFNNEQADNMTMSLKVTFIDDPSADKQIAVINTTGSFMKANPTL
SDAPVDGYPIPGASVTLRYPSQYDIAMNLQDNTSRFFHVAPTNAVEETTVTSSVSYQLGGSIKASV
TPSGPSGESGATGQVTWSDSVSYKQTSYKTNLIDQTNKHVKWNVFFNGYNNQNWGIYTRDSYHALY
GNQLFMYSRTYPHETDARGNLVPMNDLPALTNSGFSPGMIAVVISEKDTEQSSIQVAYTKHADDYT
LRPGFTFGTGNWVGNNIKDVDQKTFNKSFVLDWKNKKLVEKK
SEQ ID NO: 2
ATGCAAACCACCTCCCAAGTCGTCACGGACATCGGTCAGAACGCTAAAACCCATACCAGCTACAAT
ACCTTCAATAACGAACAAGCAGATAACATGACCATGAGCCTGAAAGTCACGTTTATTGATGACCCG
TCTGCAGATAAGCAGATTGCTGTTATCAACACCACGGGCTCATTCATGAAAGCAAATCCGACGCTG
TCGGATGCTCCGGTGGACGGTTATCCGATTCCGGGTGCTAGTGTTACCCTGCGTTATCCGTCCCAG
TACGATATCGCGATGAACCTGCAAGACAATACCAGTCGCTTTTTCCATGTGGCGCCGACGAATGCC
GTTGAAGAAACCACGGTCACCAGCTCTGTGAGCTATCAGCTGGGCGGTAGCATCAAAGCCTCTGTG
ACCCCGTCTGGTCCGAGTGGTGAATCCGGTGCAACCGGTCAAGTCACGTGGTCAGATAGCGTGAGC
TATAAACAGACCAGCTACAAGACGAACCTGATTGACCAAACCAATAAACACGTTAAGTGGAACGTC
TTTTTCAATGGCTATAACAATCAGAACTGGGGTATCTACACCCGTGATAGTTATCATGCCCTGTAC
GGCAATCAACTGTTTATGTATTCCCGTACCTACCCGCACGAAACGGATGCGCGCGGTAACCTGGTG
CCGATGAATGACCTGCCGGCCCTGACCAACTCAGGCTTCTCGCCGGGTATGATTGCAGTGGTTATC
TCTGAAAAAGATACCGAACAGAGTTCCATTCAAGTTGCGTATACCAAGCATGCCGATGACTACACG
CTGCGTCCGGGTTTTACCTTCGGTACGGGTAATTGGGTTGGTAACAATATCAAAGATGTCGACCAG
AAAACCTTCAATAAATCGTTCGTGCTGGACTGGAAAAATAAGAAACTGGTGGAAAAGAAATAATGA
SEQ ID NO: 3

| | |
|---|---|
| 1 | MMNDGKQQST FLFHDYETFG THPALDRPAQ FAAIRTDSEF NVIGEPEVFY |
| 51 | CKPADDYLPQ PGAVLITGIT PQEARAKGEN EAAFAARIHS LFTVPKTCIL |
| 101 | GYNNVRFDDE VTRNIFYRNF YDPYAWSWQH DNSRWDLLDV MRACYALRPE |
| 151 | GINWPENDDG LPSFRLEHLT KANGIEHSNA HDAMADVYAT IAMAKLVKTR |
| 201 | QPRLFDYLFT HRNKHKLMAL IDVPQMKPLV HVSGMFGAWR GNTSWVAPLA |
| 251 | WHPENRNAVI MVDLAGDISP LLELDSDTLR ERLYTAKTDL GDNAAVPVKL |
| 301 | VHINKCPVLA QANTLRPEDA DRLGINRQHC LDNLKILREN PQVREKVVAI |
| 351 | FAEAEPFTPS DNVDAQLYNG FFSDADRAAM KIVLETEPRN LPALDITFVD |
| 401 | KRIEKLLFNY RARNFPGTLD YAEQQRWLEH RRQVFTPEFL QGYADELQML |
| 451 | VQQYADDKEK VALLKALWQY AEEIV SGSGH HHHHH |

SEQ ID NO: 4

| | |
|---|---|
| 1 | MKFVSFNING LRARPHQLEA IVEKHQPDVI GLQETKVHDD MFPLEEVAKL GYNVFYHGQK GHYGVALLTK |
| 71 | ETPIAVRRGF PGDDEEAQRR IIMAEIPSLL GNVTVINGYF PQGESRDHPI KFPAKAQFYQ NLQNYLETEL |
| 141 | KRDNPVLIMG DMNISPTDLD IGIGEENRKR WLRTGKCSFL PEEREWMDRL MSWGLVDTFR HANPQTADRF |
| 211 | SWFDYRSKGF DDNRGLRIDL LLASQPLAEC CVETGIDYEI RSMEKPSDHA PVWATFRR |

SEQ ID NO: 5

| | |
|---|---|
| 1 | MERRKEDLDP PLALLPLKGL REAAALLEEA LRQGKRIRVH GDYDADGLTG TAILVRGLAA LGADVHPFIP |
| 71 | HRLEEGYGVL MERVPEHLEA SDLFLTVDCG ITNHAELREL LENGVEVIVT DHHTPGKTPP PGLVVHPALT |
| 141 | PDLKEKPTGA GVAFLLLWAL HERLGLPPPL EYADLAAVGT IADVAPLWGW NRALVKEGLA RIPASSWVGL |
| 211 | RLLAEAVGYT GKAVEVAFRI APRINAASRL GEAEKALRLL LTDDAAEAQA LVGELHRLNA RRQTLEEAML |
| 281 | RKLLPQADPE AKAIVLLDPE GHPGVMGIVA SRILEATLRP VFLVAQGKGT VRSLAPISAV EALRSAEDLL |
| 351 | LRYGGHKEAA GFAMDEALFP AFKARVEAYA ARFPDPVREV ALLDLLPEPG LLPQVFRELA LLEPYGEGNP |
| 421 | EPLFL |

SEQ ID NO: 6

| | |
|---|---|
| 1 | MTPDIILQRT GIDVRAVEQG DDAWHKLRLG VITASEVHNV IAKPRSGKKW PDMKMSYFHT LLAEVCTGVA |
| 71 | PEVNAKALAW GKQYENDART LFEFTSGVNV TESPITYRDE SMRTACSPDG LCSDGNGLEL KCPFTSRDFM |
| 141 | KFRLGGFEAI KSAYMAQVQY SMWVTRKNAW YFANYDPRMK REGLHYVVIE RDEKYMASFD EIVPEFIEKM |
| 211 | DEALAEIGFV FGEQWR |

SEQ ID NO: 7
MKHMPRKMYSCAFETTTKVEDCRVWAYGYMNIEDHSEYKIGNSLDEFMAWVLKVQADLYFHNLKFD
GAFIINWLERNGFKWSADGLPNTYNTIISRMGQWYMIDICLGYKGKRKIHTVIYDSLKKLPFPVKK
IAKDFKLTVLKGDIDYHKERPVGYKITPEEYAYIKNDIQIIAEALLIQFKQGLDRMTAGSDSLKGF
KDIITTKKFKKVFPTLSLGLDKEVRYAYRGGFTWLNDRFKEKEIGEGMVFDVNSLYPAQMYSRLLP
YGEPIVFEGKYVWDEDYPLHIQHIRCEFELKEGYIPTIQIKRSRFYKGNEYLKSSGGEIADLWLSN
VDLELMKEHYDLYNVEYISGLKFKATTGLFKDFIDKWTYIKTTSEGAIKQLAKLMLNSLYGKFASN
PDVTGKVPYLKENGALGFRLGEEETKDPVYTPMGVFITAWARYTTITAAQACYDRIIYCDTDSIHL
TGTEIPDVIKDIVDPKKLGYWAHESTFKRAKYLRQKTYIQDIYMKEVDGKLVEGSPDDYTDIKFSV
KCAGMTDKIKKEVTFENFKVGFSRKMKPKPVQVPGGVVLVDDTFTIKSGGSAWSHPQFEKGGGSGG
GSGGSAWSHPQFEK
SEQ ID NO: 8
MMIRELDIPRDIIGFYEDSGIKELYPPQAEAIEMGLLEKKNLLAAIPTASGKTLLAELAMIKAIRE
GGKALYIVPLRALASEKFERFKELAPFGIKVGISTGDLDSRADWLGVNDIIVATSEKTDSLLRNGT
SWMDEITTVVVDEIHLLDSKNRGPTLEVTITKLMRLNPDVQVVALSATVGNAREMADWLGAALVLS
EWRPTDLHEGVLFGDAINFPGSQKKIDRLEKDDAVNLVLDTIKAEGQCLVFESSRRNCAGFAKTAS
SKVAKILDNDIMIKLAGIAEEVESTGETDTAIVLANCIRKGVAFHHAGLNSNHRKLVENGFRQNLI
KVISSTPTLAAGLNLPARRVIIRSYRRFDSNFGMQPIPVLEYKQMAGRAGRPHLDPYGESVLLAKT
YDEFAQLMENYVEADAEDIWSKLGTENALRTHVLSTIVNGFASTRQELFDFFGATFFAYQQDKWML
EEVINDCLEFLIDKAMVSETEDIEDASKLFLRGTRLGSLVSMLYIDPLSGSKIVDGFKDIGKSTGG
NMGSLEDDKGDDITVTDMTLLHLVCSTPDMRQLYLRNTDYTIVNEYIVAHSDEFHEIPDKLKETDY
EWFMGEVKTAMLLEEWVTEVSAEDITRHFNVGEGDIHALADTSEWLMHAAAKLAELLGVEYSSHAY
SLEKRIRYGSGLDLMELVGIRGVGRVRARKLYNAGFVSVAKLKGADISVLSKLVGPKVAYNILSGI
GVRVNDKHENSAPISSNTLDTLLDKNQKTENDFQ
SEQ ID NO: 9
MRISELDIPRPAIEFLEGEGYKKLYPPQAAAAKAGLTDGKSVLVSAPTASGKTLIAAIAMISHLSR
NRGKAVYLSPLRALAAEKFAEFGKIGGIPLGRPVRVGVSTGDFEKAGRSLGNNDILVLINERMDSL
IRRRPDWMDEVGLVIADEIHLIGDRSRGPTLEMVLTKLRGLRSSPQVVALSATISNADEIAGWLDC
TLVHSTWRPVPLSEGVYQDGEVAMGDGSRHEVAATGGGPAVDLAAESVAEGGQSLIFADTRARSAS
LAAKASAVIPEAKGADAAKLAAAAKKIISSGGETKLAKTLAELVEKGAAFHHAGLNQDCRSVVEEE
FRSGRIRLLASTPTLAAGVNLPARRVVISSVMRYNSSSGMSEPISILEYKQLCGRAGRPQYDKSGE
AIVVGGVNADEIFDRYIGGEPEPIRSAMVDDRALRIHVLSLVTTSPGIKEDDVTEFFLGTLGGQQS
GESTVKFSVAVALRFLQEEGMLGRRGGRLAATKMGRLVSRLYMDPMTAVTLRDAVGEASPGRMHTL
GFLHLVSECSEFMPRFALRQKDHEVAEMMLEAGRGELLRPVYSYECGRGLLALHRWIGESPEAKLA
EDLKFESGDVHRMVESSGWLLRCIWEISKHQERPDLLGELDVLRSRVAYGIKAELVPLVSIKGIGR
VRSRRLFRGGIKGPGDLAAVPVERLSRVEGIGATLANNIKSQLRKGG
SEQ ID NO: 10
MKVDELPVDERLKAVLKERGIEELYPPQAEALKSGALEGRNLVLAIPTASGKTLVSEIVMVNKLIQ
EGGKAVYLVPLKALAEEKYREFKEWEKLGLKVAATTGDYDSTDDWLGRYDIIVATAEKFDSLLRHG
ARWINDVKLVVADEVHLIGSYDRGATLEMILTHMLGRAQILALSATVGNAEELAEWLDASLVVSDW
RPVQLRRGVFHLGTLIWEDGKVESYPENWYSLVVDAVKRGKGALVFVNTRRSAEKEALALSKLVSS
HLTKPEKRALESLASQLEDNPTSEKLKRALRGGVAFHHAGLSRVERTLIEDAFREGLIKVITATPT
LSAGVNLPSFRVIIRDTKRYAGFGWTDIPVLEIQQMMGRAGRPRYDKYGEAIIVARTDEPGKLMER
YIRGKPEKLFSMLANEQAFRSQVLALITNFGIRSFPELVRFLERTFYAHQRKDLSSLEYKAKEVVY
FLIENEFIDLDLEDRFIPLPFGKRTSQLYIDPLTAKKFKDAFPAIERNPNPFGIFQLIASTPDMAT
LTARRREMEDYLDLAYELEDKLYASIPYYEDSRFQGFLGQVKTAKVLLDWINEVPEARIYETYSID
PGDLYRLLELADWLMYSLIELYKLFEPKEEILNYLRDLHLRLRHGVREELLELVRLPNIGRKRARA
LYNAGERSVEAIANAKPAELLAVEGIGAKILDGIYRHLGIEKRVTEEKPKRKGTLEDFLR
SEQ ID NO: 11
MEIASLPLPDSFIRACHAKGIRSLYPPQAECIEKGLLEGKNLLISIPTASGKTLLAEMAMWSRIAA
GGKCLYIVPLRALASEKYDEFSKKGVIRVGIATGDLDRTDAYLGENDIIVATSEKTDSLLRNRTPW
LSQITCIVLDEVHLIGSENRGATLEMVITKLRYTNPVMQIIGLSATIGNPAQLAEWLDATLITSTW
RPVDLRQGVYYNGKIRFSDSERPIQGKTKHDDLNLCLDTIEEGGQCLVFVSSRRNAEGFAKKAAGA
LKAGSPDSKALAQELRRLRDRDEGNVLADCVERGAAFHHAGLIRQERTIIEEGFRNGYIEVIAATP
TLAAGLNLPARRVIIRDYNRFASGLGMVPIPVGEYHQMAGRAGRPHLDPYGEAVLLAKDAPSVERL
FETFIDAEAERVDSQCVDDASLCAHILSLIATGFAHDQEALSSFMERTFYFFQHPKTRSLPRLVAD
AIRFLTTAGMVEERENTLSATRLGSLVSRLYLNPCTARLILDSLKSCKTPTLIGLLHVICVSPDMQ
RLYLKAADTQLLRTFLFKHKDDLILPLPFEQEEEELWLSGLKTALVLTDWADEFSEGMIEERYGIG
AGDLYNIVDSGKWLLHGTERLVSVEMPEMSQVVKTLSVRVHHGVKSELLPLVALRNIGRVRARTLY
NAGYPDPEAVARAGLSTIARIIGEGIARQVIDEITGVKRSGIHSSDDDYQQKTPELLTDIPGIGKK
MAEKLQNAGIITVSDLLTADEVLLSDVLGAARARKVLAFLSNSEKENSSSDKTEEIPDTQKIRGQS
SWEDFGC
SEQ ID NO: 12

| | |
|---|---|
| 1 | MMSIAQVRSA GSAGNYYTDK DNYYVLGSMG ERWAGKGAEQ LGLQGSVDKD VFTRLLEGRL |
| 61 | PDGADLSRMQ DGSNKHRPGY DLTFSAPKSV SMMAMLGGDK RLIDAHNQAV DFAVRQVEAL |
| 121 | ASTRVMTDGQ SETVLTGNLV MALFNHDTSR DQEPQLHTHA VVANVTQHNG EWKTLSSDKV |
| 181 | VEAFSGRSQA ANQIAFGRLY REKLKEQVEA LGYETEVVGK HGMWEMPGVP GKTGFIENVY |
| 241 | YRDAADQRTE LKSRDVAALD TRKSKQHVDP EIRMAEWMQT LKETGFDIRA IREAVGEDAS |
| 301 | NGVIERARAG QDGPDVQQAV TQAIAGLSER KVQFTYTDVL ARTVGILPPE IRTQAPGPAS |
| 361 | KSVPRTAGYS IPLDREKGLF TSGIHVLDEL SVRALSRDIM KQNRVTVHPE IDEAISREQL |
| 421 | RSQMNLKQDE PSLAIVSGQG GAAGQRERVA ELVMMAREQG REVQIIAADR DAVSVLAQDR |
| 481 | QVLITDSGQR RQLLEGMAFT PGSTVIVDQG EKLSLKETLT LLDGAARHNV RLSGELITGR |
| 541 | TGTGSALMAM KDAGVNTYRW QGGEQRPATI ISEPDRNVRY ARLAGDFAAS VKAGEESVAQ |
| 601 | RPGMVMEQWN TQAIRSELKT QGVLGHPEVT MTALSPVWLD SRSRYLRDMY VSGVREQAIL |
| 661 | PVADGERLRV IDRVTAQSHS LTLRDAQGET QVVRISSLDS SWSLFRPEKM PETRSHDRYV |
| 721 | ENGWVETPGH GGDRLQVASV SEDAMTVVVP GRAEPASLPV SDSPFTALKL TGKIPGLRVS |
| 781 | PSFTVVSEQI SVTQMAMDNA TLNGLARSGR DVRLYSSLDE TRTAEKLARH SVSDSATVFA |
| 841 | AKSFAAEGTG TAISLQKAGL HTPAQQAIHL ALPVLESKNL AFSMVDLLTE KARAGETLLE |
| 901 | KEAVTPLMER QIKRGDLLYV DVAKGYGTGL LVSRASYEAE KSILRHILEG FTELGGEINA |
| 961 | MLPASERPRV SGQRAATRMI LETSDRFTVV QGYAGVGKTT QFRAVMSAVN VPGELMETLT |
| 1021 | ESSMVGNTEM GEMRSAGVDA QTLASFLHDT QLQQRSGETP DESNTLFLLD VGLGPTHRAV |
| 1081 | RQTPELREAV GGRAVASGDT DQLQAIAPGQ SFRLQQTRSA ADVVIMKEIV ARAYALIAAG |
| 1141 | QQKAMLKGEA ALSGLESVKP SQVPRLEGAW APEHSVTEFS HSQEAKLAEA YSLINRDVER |
| 1201 | FPDIPMTLYE AIVRDYTGRT PEAREQTLIV THLNEDRRVL NSMIHDAREK AGELGKEQVM |
| 1261 | DAEGNTRLIS DGELRRLSTW EKNPDALALV DNVYHRIAGI SKDDGLITLQ VPVLNTANIR |
| 1321 | SVTLSDGQQT LYTPDKIRVG TGDRMRFTKS DRERGYVANS VWTVTAVSGD PREAVAEGVT |
| 1381 | SAYVALSRMK EQHIDLAYAI TAHGAQGASE TFAIALEGTE GNRKLMAGFE RVIRPGQERA |
| 1441 | LRDVAAGRAV QGWTDAINNA VQKGTAHDVL EPKPDREVMN AQRLFSTARE QHVQVYTDNR |
| 1501 | GNGLRGFSGE SPARFIAPGR KYPQPYVALP AFDRNGKSAG IWLNPLTTDD LRQAGLAGGD |
| 1561 | GRPWNPGAIT FVALQGSRNG ESLLADNMQD GVRIARDNPD SGVVVRIAGE GRVKGSGDAQ |
| 1621 | GGRVWGDIPD NSVQPGAGNG EPVTAEVLAQ RQAEEAIRRE TERRADEIVR KMAENKPDLP |
| 1681 | DGKTELAVRD IAGQERDRSA ISERETALPE SVLRESQRER EAVREVAREN LLQERLQQME |
| 1741 | RDMVRDLQKE KTLGGD |

SEQ ID NO: 13

| | |
|---|---|
| 1 | MSDKPAFMKY FTQSSCYPNQ QEAMDRIHSA LMQQQLVLFE GACGTGKTLS ALVPALHVGK |
| 61 | MLGKTVIIAT NVHQQMVQFI NEARDIKKVQ DVKVAVIKGK TAMCPQEADY EECSVKRENT |
| 121 | FELMETEREI YLKRQELNSA RDSYKKSHDP AFVTLRDELS KEIDAVEEKA RGLRDRACND |
| 181 | LYEVLRSDSE KFREWLYKEV RSPEEINDHA IKDGMCGYEL VKRELKHADL LICNYHHVLN |
| 241 | PDIFSTVLGW IEKEPQETIV IFDEAHNLES AARSHSSLSL TEHSIEKAIT ELEANLDLLA |
| 301 | DDNIHNLFNI FLEVISDTYN SRFKFGERER VRKNWYDIRI SDPYERNDIV RGKFLRQAKG |
| 361 | DFGEKDDIQI LLSEASELGA KLDETYRDQY KKGLSSVMKR SHIRYVADFM SAYIELSHNL |
| 421 | NYYPILNVRR DMNDEIYGRV ELFTCIPKNV TEPLENSLES VILMSATLHP FEMVKKTLGI |
| 481 | TRDTCEMSYG TSFPEEKRLS IAVSIPPLFA KNRDDRHVTE LLEQVLLDSI ENSKGNVILF |
| 541 | FQSAFEAKRY YSKIEPLVNV PVFLDEVGIS SQDVREEFFS IGEENGKAVL LSYLWGTLSE |
| 601 | GIDYRDGRGR TVIIIGVGYP ALNDRMNAVE SAYDHVFGYG AGWEFAIQVP TIRKIRQAMG |
| 661 | RVVRSPTDYG ARILLDGRFL TDSKKRFGKF SVFEVFPPAE RSEFVDVDPE KVKYSLMNFF |
| 721 | MDNDEQ |

SEQ ID NO: 14
MTFDDLTEGQKNAFNIVMKAIKEKKHHVTINGPAGTGKTTLTKFIIEALISTGETGIILAAPTHAA
KKILSKLSGKEASTIHSILKINPVTYEENVLFEQKEVPDLAKCRVLICDEVSMYDRKLFKILLSTI
PPWCTIIGIGDNKQIRPVDPGENTAYISPFFTHKDFYQCELTEVKRSNAPIIDVATDVRNGKWIYD
KVVDGHGVRGFTGDTALRDFMVNYFSIVKSLDDLFENRVMAFTNKSVDKLNSIIRKKIFETDKDFI
VGEIIVMQEPLFKTYKIDGKPVSEIIFNNGQLVRIIEAEYTSTFVKARGVPGEYLIRHWDLTVETY
GDDEYYREKIKIISSDEELYKFNLFLGKTAETYKNWNKGGKAPWSDFWDAKSQFSKVKALPASTFH
KAQGMSVDRAFIYTPCIHYADVELAQQLLYVGVTRGRYDVFYV
SEQ ID NO: 15

| | |
|---|---|
| 1 | MLSVANVRSP SAAASYFASD NYYASADADR SGQWIGDGAK RLGLEGKVEA RAFDALLRGE |
| 61 | LPDGSSVGNP GQAHRPGTDL TFSVPKSWSL LALVGKDERI IAAYREAVVE ALHWAEKNAA |
| 121 | ETRVVEKGMV VTQATGNLAI GLFQHDTNRN QEPNLHFHAV IANVTQGKDG KWRTLKNDRL |
| 181 | RRKEVLEARR AMARFRVAVE KLGYEPGPVL KHGNFEARGI SREQVMAFST WQLNTTLNSI |
| 241 | TKALGQGMEA ALDTRASKEG IEDRATLSKQ WSEAAQSIGL DLKPLVDRAR GPGLDAGRIA |
| 301 | AVRHLSQREA RAWLSRFAAH VRGDPADPLV PPSVLKQDRQ TIAAAQAVAS TRIGSLVERG |
| 361 | DAVVTEQRIL ALDFGLPTTI ADVEKRTRAL VRSGDLIAGK GEHKGWLASR AFERTALYKA |
| 421 | DRTIAVQGIA SPAITPQKAA ASVQAAALTG QGFRLNEGQL AAARLILISK SEVAAGKGDS |
| 481 | GGWNKLLDDP VAEVLRDEGH PVIGLAIQNT LVQMLERDTG IGSQTLARFL GAGKSSVLKP |
| 541 | DRKQLGAVDA ASLKDHVLVL DEASMVSNED KEKLVRLANL AGVHRLVLIG GNVALRAEAQ |
| 601 | TVEARGDGAQ GIARAEMATN LRARDPVVRE AQAAAQAGDV RKALRHLKSH GKPFALLQRA |
| 661 | LEVLDRVNTT KETRARTSIY ASGRAIRSAV NAAVQQGLLA SREIGPAKMK VAAETWLALD |
| 721 | GKRFREDPAR RAGRVLEVSR KQQALGLFIG EYRVIGQDRK GKLVEVEDKR REELRHLPAY |
| 781 | GKVTFETSKG TLLEPRKLEI HEGDRIRWTR NDHRRGLENA DQARVVEIAN IRAGKGDDNL |
| 841 | FLVTVTRLRD MLKRIDLAYA LNVHMAQGLT SDRGIAVMDS RERNLSNQKT DLVELKKDDP |
| 901 | ANKAEKELTR KLGAAVARNK GEKASAIEVT GSVKPTATKG SGVDQPKSVE HLTLVVDSAD |
| 961 | SKSKTLDFGI |

SEQ ID NO: 16
ATGAAACACATGCCGCGTAAAATGTATAGCTGCGCGTTTGAAACCACGACCAAAGTGGAAGATTGT
CGCGTTTGGGCCTATGGCTACATGAACATCGAAGATCATTCTGAATACAAAATCGGTAACAGTCTG
GATGAATTTATGGCATGGGTGCTGAAAGTTCAGGCGGATCTGTACTTCCACAACCTGAAATTTGAT
GGCGCATTCATTATCAACTGGCTGGAACGTAATGGCTTTAAATGGAGCGCGGATGGTCTGCCGAAC
ACGTATAATACCATTATCTCTCGTATGGGCCAGTGGTATATGATTGATATCTGCCTGGGCTACAAA
GGTAAACGCAAAATTCATACCGTGATCTATGATAGCCTGAAAAAACTGCCGTTTCCGGTGAAGAAA
ATTGCGAAAGATTTCAAACTGACGGTTCTGAAAGGCGATATTGATTATCACAAAGAACGTCCGGTT
GGTTACAAAATCACCCCGGAAGAATACGCATACATCAAAAACGATATCCAGATCATCGCAGAAGCG
CTGCTGATTCAGTTTAAACAGGGCCTGGATCGCATGACCGCGGGCAGTGATAGCCTGAAAGGTTTC
AAAGATATCATCACGACCAAAAAATTCAAAAAAGTGTTCCCGACGCTGAGCCTGGGTCTGGATAAA
GAAGTTCGTTATGCCTACCGCGGCGGTTTTACCTGGCTGAACGATCGTTTCAAAGAAAAAGAAATT
GGCGAGGGTATGGTGTTTGATGTTAATAGTCTGTATCCGGCACAGATGTACAGCCGCCTGCTGCCG
TATGGCGAACCGATCGTGTTCGAGGGTAAATATGTTTGGGATGAAGATTACCCGCTGCATATTCAG
CACATCCGTTGTGAATTTGAACTGAAAGAAGGCTATATTCCGACCATTCAGATCAAACGTAGTCGC
TTCTATAAGGGTAACGAATACCTGAAAAGCTCTGGCGGTGAAATCGCGGATCTGTGGCTGAGTAAC
GTGGATCTGGAACTGATGAAAGAACACTACGATCTGTACAACGTTGAATACATCAGCGGCCTGAAA
TTTAAAGCCACGACCGGTCTGTTCAAAGATTTCATCGATAAATGGACCTACATCAAAACGACCTCT
GAAGGCGCGATTAAACAGCTGGCCAAACTGATGCTGAACAGCCTGTATGGCAAATTCGCCTCTAAT
CCGGATGTGACCGGTAAAGTTCCGTACCTGAAAGAAAATGGCGCACTGGGTTTTCGCCTGGGCGAA
GAAGAAACGAAAGATCCGGTGTATACCCCGATGGGTGTTTTCATTACGGCCTGGGCACGTTACACG
ACCATCACCGCGGCCCAGGCATGCTATGATCGCATTATCTACTGTGATACCGATTCTATTCATCTG
ACGGGCACCGAAATCCCGGATGTGATTAAAGATATCGTTGATCCGAAAAAACTGGGTTATTGGGCC
CACGAAAGTACGTTTAAACGTGCAAAATACCTGCGCCAGAAAACCTACATCCAGGATATCTACATG
AAAGAAGTGGATGGCAAACTGGTTGAAGGTTCTCCGGATGATTACACCGATATCAAATTCAGTGTG
AAATGCGCCGGCATGACGGATAAAATCAAAAAAGAAGTGACCTTCGAAAACTTCAAAGTTGGTTTC
AGCCGCAAAATGAAACCGAAACCGGTGCAGGTTCCGGGCGGTGTGGTTCTGGTGGATGATACGTTT
ACCATTAAATCTGGCGGTAGTGCGTGGAGCCATCCGCAGTTCGAAAAAGGCGGTGGCTCTGGTGGC
GGTTCTGGCGGTAGTGCCTGGAGCCACCCGCAGTTTGAAAAATAATAA

The following are numbered aspects of the invention:

1. A mutant Cytotoxin K monomer comprising a variant of the amino acid sequence of SEQ ID NO: 1; wherein the monomer is capable of forming a pore; and wherein the variant comprises one or more modifications at one or more positions in the region of SEQ ID NO: 1 between about S100 and about K170 which alter the ability of the monomer to interact with an analyte.

2. A monomer according to aspect 1, wherein the variant has at least 70% identity to the amino acid sequence of SEQ ID NO: 1.

3. A monomer according to aspect 1 or aspect 2, wherein the one or more modifications each independently (a) alter the size of the amino acid residue at the modified position; (b) alter the net charge of the amino acid residue at the modified position; (c) alter the hydrogen bonding characteristics of the amino acid residue at the modified position; (d) introduce to or remove from the amino acid residue at the modified position one or more chemical groups that interact through delocalized electron pi systems and/or (e) alter the structure of the amino acid residue at the modified position.

4. A monomer according to any one of the preceding aspects, wherein said monomer is capable of forming a pore having a solvent-accessible channel from a first opening to a second opening of said pore; the solvent-accessible channel comprising at least one constriction; and wherein the one or more modifications are made to amino acids in said constriction.

5. A monomer according to aspect 4, wherein said modifications alter the interaction of the constriction with an analyte as the analyte moves through the pore.

6. A monomer according to aspect 4 or aspect 5, wherein the one or more modifications (a) alter the size of the constriction; (b) alter the net charge of the constriction; (c) alter the hydrogen bonding characteristics of the amino acid residues in the constriction; (d) introduce to or remove from the constriction one or more chemical groups that interact through delocalized electron pi systems and/or (e) alter the structure of the constriction.

7. A monomer according to any one of the preceding aspects, wherein the variant comprises one or more modifications at one or more positions in the region of SEQ ID NO: 1 between about V111 and about T158.

8. A monomer according to any one of the preceding aspects, wherein the variant comprises one or more modifications in the region of SEQ ID NO: 1 between about V111 and about S131; and/or between about S135 and about T158.

9. A monomer according to any one of the preceding aspects, wherein the variant comprises one or more modifications in the region of SEQ ID NO: 1 between about S119 and about G126, preferably between S121 and G125; and/or between about A143 and about S150, preferably between T144 and T148.

10. A monomer according to any one of the preceding aspects, wherein the variant comprises one or more modifications in the region of SEQ ID NO: 1 between about G126 and about V132, preferably between S127 and S131 and/or between about P137 and about A143, preferably between S138 and G142.

11. A monomer according to any one of the preceding aspects, wherein the variant comprises one or more modifications in the region of SEQ ID NO: 1 between about N109 and about T117, preferably between V111 and T115; and/or between about S152 and about Y160, preferably between S154 and T158.

12. A monomer according to any one of the preceding aspects, comprising a modification at one or more of the following positions of SEQ ID NO: 1: E113, T115, T117, S119, S121, Q123, G125, S127, K129, S131, V132, T133, P134, S135, G136, P137, S138, E140, G142, T144, Q146, T148, S150, S152, S154 and K156.

13. A monomer according to any one of the preceding aspects, wherein the variant independently comprises one or more amino acid substitutions, additions and/or deletions at said one or more positions.

14. A monomer according to any one of the preceding aspects, wherein the variant comprises one or more amino acid substitutions and the amino acid(s) substituted into the variant are selected from aspartate, glutamate, serine, threonine, asparagine, glutamine, glycine, alanine, valine, leucine, isoleucine, cysteine, arginine, lysine and phenylalanine.

15. A monomer according to any one of the preceding aspects, comprising one or more modifications selected from:
E113S/T/N/Q/G/A/V/L/I/C/R/K/F/Y
T115S/N/Q/G/A/V/L/I/C/R/K/F
T117S/N/Q/G/A/V/L/I/C/R/K/F
S119T/N/Q/G/A/V/L/I/C/R/K/F
S121T/N/Q/G/A/V/L/I/C/R/K/F
Q123S/T/N/G/A/V/L/I/C/R/K/F/M/Y
G125S/T/N/Q/A/V/L/I/C/R/K/F
S127T/N/Q/G/A/V/L/I/C/R/K/F
K129S/T/N/Q/G/A/V/L/I/C/R/F/Y
S131T/N/Q/G/A/V/L/I/C/R/K/F
V132S/T/N/Q/G/A/L/I/C/R/K/F
T133S/N/Q/G/A/V/L/I/C/R/K/F
P134S/T/N/Q/G/A/V/L/I/C/R/K/F
S135T/N/Q/G/A/V/L/I/C/R/K/F
G136S/T/N/Q/A/V/L/I/C/R/K/F
P137S/T/N/Q/G/A/V/L/I/C/R/K/F
S138T/N/Q/G/A/V/L/I/C/R/K/F
E140S/T/N/Q/G/A/V/L/I/C/R/K/F
G142S/T/N/Q/A/V/L/I/C/R/K/F
T144S/N/Q/G/A/V/L/I/C/R/K/F
Q146S/T/N/G/A/V/L/I/C/R/K/F/M/Y
T148S/N/Q/G/A/V/L/I/C/R/K/F
S150T/N/Q/G/A/V/L/I/C/R/K/F
S152T/N/Q/G/A/V/L/I/C/R/K/F
S154T/N/Q/G/A/V/L/I/C/R/K/F; and
K156S/T/N/Q/G/A/V/L/I/C/R/F.

16. A monomer according to any one of the preceding aspects, comprising a modification at one or more of: E113, Q123, K129, E140, Q146, and K156.

17. A monomer according to any one of the preceding aspects, comprising modifications at Q123 and/or Q146.

18. A monomer according to any one of the preceding aspects, comprising modifications at K129 and/or E140.

19. A monomer according to any one of the preceding aspects, comprising modifications at E113 and/or K156.

20. A monomer according to any one of the preceding aspects, comprising modifications at:
(i) Q123 and/or Q146; and (ii) K129 and/or E140.
(i) E113 and/or K156; and (ii) Q123 and/or Q146; or
(i) E113 and/or K156; and (ii) K129 and/or E140.

21. A monomer according to any one of the preceding aspects, comprising modifications at (i) E113 and/or K156; (ii) Q123 and/or Q146; and (iii) K129 and/or E140.

22. A monomer according to any one of the preceding aspects, containing one or more of: E113S/N/Y/K/R; Q123S/A/N/M/Y/G/K/R; K129S/N/Y; E140S/N/K/R; Q146S/A/N/M/K/R/G/Y and K156S/N.

23. A monomer according to any one of the preceding aspects, wherein said monomer is chemically modified.

24. A monomer according to aspect 23, wherein said monomer is chemically modified by attachment of a molecule to one or more cysteines, attachment of a molecule to one or more lysines, attachment of a molecule to one or more non-natural amino acids, enzyme modification of an epitope or modification of a terminus.

25. A monomer according to any one of the preceding aspects, wherein said monomer is capable of forming a heptameric pore.

26. A construct comprising two or more covalently attached monomers derived from Cytotoxin K, wherein at least one of the monomers is a mutant Cytotoxin K monomer as defined in any one of the preceding aspects.

27. A construct according to aspect 26, wherein the monomers are genetically fused or are attached via a linker.

28. A polynucleotide which encodes a mutant Cytotoxin K monomer according to any one of aspects 1-25 or a construct according to aspect 26-27

29. A homo-oligomeric pore comprising a plurality of mutant monomers according to any one of aspects 1-25; wherein said pore is preferably a heptameric pore.

30. A hetero-oligomeric pore comprising at least one mutant monomer according to any one of aspects 1-25; wherein said pore is preferably a heptameric pore.

31. A pore comprising at least one construct according to aspects 26-27.

32. A construct according to aspects 26 or 27, or a pore according to any one of aspects 29-31, wherein at least one monomer in said construct or pore is a monomer of SEQ ID NO: 1.

33. A membrane comprising a pore according to any one of the aspects 29-31.

34. An array comprising a plurality of membranes according to aspect 33.

35. A device comprising the array of aspect 34, means for applying a potential across the membranes and means for detecting electrical or optical signals across the membranes.

36. A method of characterising a target analyte, comprising:
(a) contacting the target analyte with a pore according to any one of aspects 29-31 such that the target analyte moves with respect to the pore; and
(b) taking one or more measurements characteristic of the analyte as the analyte moves with respect to the pore,
thereby characterising the target analyte.

37. A method according to aspect 36, wherein the target analyte is a metal ion, an inorganic salt, a polymer, an amino acid, a peptide, a polypeptide, a protein, a nucleotide, an oligonucleotide, a polynucleotide, an oligosaccharide.

38. A method according to aspect 37, wherein the target analyte is or comprises a polypeptide or a polynucleotide.

39. A method according to aspect 37 or aspect 38, wherein the target analyte comprises a polynucleotide and said method comprises (i) contacting the polynucleotide with a polynucleotide binding protein capable of controlling the movement of the polynucleotide with respect to the pore; and (ii) taking one or more measurements characteristic of the polynucleotide as the polynucleotide moves with respect to the pore.

40. Use of a pore according to any one of aspects 29-31 to characterise a target analyte.

41. A method of characterising a target polypeptide, comprising:
(a) contacting the target polypeptide with a Cytotoxin K pore such that the target analyte moves with respect to the pore; and
(b) taking one or more measurements characteristic of the polypeptide as the polypeptide moves with respect to the pore,
thereby characterising the target polypeptide.

42. A method according to aspect 41, wherein said method comprises (i) contacting the polypeptide with a polypeptide handling enzyme capable of controlling the movement of the polypeptide with respect to the pore; and (ii) taking one or more measurements characteristic of the polypeptide as the polypeptide moves with respect to the pore.

43. A method according to aspect 41 or aspect 42, wherein the target analyte comprises a polynucleotide-polypeptide conjugate and said method comprises (i) contacting the conjugate with a polynucleotide binding protein capable of controlling the movement of the polynucleotide of the conjugate with respect to the pore; and (ii) taking one or more measurements characteristic of the polypeptide as the conjugate moves with respect to the pore.

44. A method according to aspect 43, wherein the Cytotoxin K pore is a pore according to any one of aspects 29-31.

45. Use of a Cytotoxin K pore to characterise a target polypeptide.

46. Use of a Cytotoxin K pore according to aspect 45, wherein the Cytotoxin K pore comprises a mutant Cytotoxin K monomer according to any one of aspects 1 to 25.

47. Use of a Cytotoxin K pore according to aspect 45 or aspect 46, wherein the Cytotoxin K pore is a pore according to any one of aspect 29-31.

48. A kit for characterising a target analyte comprising (a) a pore according to any one of aspects 29-31 and (b) a polynucleotide binding protein or polypeptide handling enzyme.

1. A method of characterising a target analyte, comprising:
(a) contacting the target analyte with a pore comprising at least one mutant Cytotoxin K monomer comprising a variant of the amino acid sequence of SEQ ID NO: 1; such that the target analyte moves with respect to the pore;
wherein the variant comprises one or more modifications at one or more positions in the region of SEQ ID NO: 1 between about S100 and about K170 which alter the ability of the monomer to interact with the analyte; and
(b) taking one or more measurements characteristic of the analyte as the analyte moves with respect to the pore,
thereby characterising the target analyte.

2. A method according to claim 1, wherein the variant has at least 70% identity to the amino acid sequence of SEQ ID NO: 1.

3. A method according to claim 1 or claim 2, wherein the one or more modifications each independently (a) alter the size of the amino acid residue at the modified position; (b) alter the net charge of the amino acid residue at the modified position; (c) alter the hydrogen bonding characteristics of the amino acid residue at the modified position; (d) introduce to or remove from the amino acid residue at the modified position one or more chemical groups that interact through delocalized electron pi systems and/or (e) alter the structure of the amino acid residue at the modified position.

4. A method according to any one of the preceding claims, wherein the pore has a solvent-accessible channel from a first opening to a second opening of said pore; the solvent-accessible channel comprising at least one constriction; and wherein the one or more modifications are made to amino acids in said constriction.

5. A method according to claim 4, wherein said modifications alter the interaction of the constriction with an analyte as the analyte moves through the pore.

6. A method according to claim 4 or claim 5, wherein the one or more modifications (a) alter the size of the constriction; (b) alter the net charge of the constriction; (c) alter the hydrogen bonding characteristics of the amino acid residues in the constriction; (d) introduce to or remove from the constriction one or more chemical groups that interact through delocalized electron pi systems and/or (e) alter the structure of the constriction.

7. A method according to any one of the preceding claims, wherein the variant comprises one or more modifications at one or more positions in the region of SEQ ID NO: 1 between about V111 and about T158.

8. A method according to any one of the preceding claims, wherein the variant comprises one or more modifications in the region of SEQ ID NO: 1 between about V111 and about S131; and/or between about S135 and about T158.

9. A method according to any one of the preceding claims, wherein the variant comprises one or more modifications in the region of SEQ ID NO: 1 between about S119 and about G126, preferably between S121 and G125; and/or between about A143 and about S150, preferably between T144 and T148.

10. A method according to any one of the preceding claims, wherein the variant comprises one or more modifications in the region of SEQ ID NO: 1 between about G126 and about V132, preferably between S127 and S131 and/or between about P137 and about A143, preferably between S138 and G142.

11. A method according to any one of the preceding claims, wherein the variant comprises one or more modifications in the region of SEQ ID NO: 1 between about N109 and about T117, preferably between V111 and T115; and/or between about S152 and about Y160, preferably between S154 and T158.

12. A method according to any one of the preceding claims, wherein the monomer comprises a modification at one or more of the following positions of SEQ ID NO: 1: E113, T115, T117, S119, S121, Q123, G125, S127, K129, S131, V132, T133, P134, S135, G136, P137, S138, E140, G142, T144, Q146, T148, S150, S152, S154 and K156.

13. A method according to any one of the preceding claims, wherein the variant independently comprises one or more amino acid substitutions, additions and/or deletions at said one or more positions.

14. A method according to any one of the preceding claims, wherein the variant comprises one or more amino acid substitutions and the amino acid(s) substituted into the variant are selected from aspartate, glutamate, serine, threonine, asparagine, glutamine, glycine, alanine, valine, leucine, isoleucine, cysteine, arginine, lysine and phenylalanine.

15. A method according to any one of the preceding claims, wherein the monomer comprises one or more modifications selected from:
E113S/T/N/Q/G/A/V/L/I/C/R/K/F/Y
T115S/N/Q/G/A/V/L/I/C/R/K/F
T117S/N/Q/G/A/V/L/I/C/R/K/F
S119T/N/Q/G/A/V/L/I/C/R/K/F
S121T/N/Q/G/A/V/L/I/C/R/K/F
Q123S/T/N/G/A/V/L/I/C/R/K/F/M/Y
G125S/T/N/Q/A/V/L/I/C/R/K/F
S127T/N/Q/G/A/V/L/I/C/R/K/F
K129S/T/N/Q/G/A/V/L/I/C/R/F/Y
S131T/N/Q/G/A/V/L/I/C/R/K/F
V132S/T/N/Q/G/A/L/I/C/R/K/F
T133S/N/Q/G/A/V/L/I/C/R/K/F
P134S/T/N/Q/G/A/V/L/I/C/R/K/F
S135T/N/Q/G/A/V/L/I/C/R/K/F
G136S/T/N/Q/A/V/L/I/C/R/K/F
P137S/T/N/Q/G/A/V/L/I/C/R/K/F
S138T/N/Q/G/A/V/L/I/C/R/K/F
E140S/T/N/Q/G/A/V/L/I/C/R/K/F
G142S/T/N/Q/A/V/L/I/C/R/K/F
T144S/N/Q/G/A/V/L/I/C/R/K/F
Q146S/T/N/G/A/V/L/I/C/R/K/F/M/Y
T148S/N/Q/G/A/V/L/I/C/R/K/F
S150T/N/Q/G/A/V/L/I/C/R/K/F
S152T/N/Q/G/A/V/L/I/C/R/K/F
S154T/N/Q/G/A/V/L/I/C/R/K/F; and
K156S/T/N/Q/G/A/V/L/I/C/R/F.

16. A method according to any one of the preceding claims, wherein the monomer comprises a modification at one or more of: E113, Q123, K129, E140, Q146, and K156.

17. A method according to any one of the preceding claims, wherein the monomer comprises modifications at Q123 and/or Q146.

18. A method according to any one of the preceding claims, wherein the monomer comprises modifications at K129 and/or E140.

19. A method according to any one of the preceding claims, wherein the monomer comprises modifications at E113 and/or K156.

20. A method according to any one of the preceding claims, wherein the monomer comprises modifications at:
(i) Q123 and/or Q146; and (ii) K129 and/or E140.
(i) E113 and/or K156; and (ii) Q123 and/or Q146; or
(i) E113 and/or K156; and (ii) K129 and/or E140.

21. A method according to any one of the preceding claims, wherein the monomer comprises modifications at (i) E113 and/or K156; (ii) Q123 and/or Q146; and (iii) K129 and/or E140.

22. A method according to any one of the preceding claims, wherein the monomer contains one or more of: E113S/N/Y/K/R; Q123S/A/N/M/Y/G/K/R; K129S/N/Y; E140S/N/K/R; Q146S/A/N/M/K/R/G/Y and K156S/N.

23. A method according to any one of the preceding claims, wherein said monomer is chemically modified.

24. A method according to claim 23, wherein said monomer is chemically modified by attachment of a molecule to one or more cysteines, attachment of a molecule to one or more lysines, attachment of a molecule to one or more non-natural amino acids, enzyme modification of an epitope or modification of a terminus.

25. A method according to any one of the preceding claims, wherein said pore is a homooligomeric pore comprising a plurality of mutant monomers as defined in any one of claims 1 to 24; wherein the pore is preferably a heptameric pore.

26. A method according to any one claims 1 to 24, wherein said pore is a heterooligomeric pore comprising at least one mutant monomer as defined in any one of claims 1 to 24; wherein the pore is preferably a heptameric pore.

27. A method according to any one of claims 1 to 24, wherein said pore comprises a construct comprising two or more covalently attached monomers derived from Cytotoxin K, wherein at least one of the monomers is a mutant Cytotoxin K monomer as defined in any one of claims 1 to 24.

28. A method according to any one of the preceding claims, wherein the target analyte is a metal ion, an inorganic salt, a polymer, an amino acid, a peptide, a polypeptide, a protein, a nucleotide, an oligonucleotide, a polynucleotide, an oligosaccharide.

29. A method according to claim 28, wherein the target analyte is or comprises a polypeptide or a polynucleotide.

30. A method according to claim 28 or claim 29, wherein the target analyte comprises a polynucleotide and said method comprises (i) contacting the polynucleotide with a polynucleotide binding protein capable of controlling the movement of the polynucleotide with respect to the pore; and (ii) taking one or more measurements characteristic of the polynucleotide as the polynucleotide moves with respect to the pore.

31. A method of characterising a target polypeptide, comprising:
(a) contacting the target polypeptide with a Cytotoxin K pore such that the target analyte moves with respect to the pore; and
(b) taking one or more measurements characteristic of the polypeptide as the polypeptide moves with respect to the pore,
thereby characterising the target polypeptide.

32. A method according to claim 31, wherein said method comprises (i) contacting the polypeptide with a polypeptide handling enzyme capable of controlling the movement of the polypeptide with respect to the pore; and (ii) taking one or more measurements characteristic of the polypeptide as the polypeptide moves with respect to the pore.

33. A method according to claim 31 or claim 32, wherein the target polypeptide is comprised in a polynucleotide-polypeptide conjugate and said method comprises (i) contacting the conjugate with a polynucleotide binding protein capable of controlling the movement of the polynucleotide of the conjugate with respect to the pore; and (ii) taking one or more measurements characteristic of the polypeptide as the conjugate moves with respect to the pore.

34. A method according to any one of claims 31 to 33, wherein the Cytotoxin K pore is a pore as defined in any one of claims 1 to 27.

35. A mutant Cytotoxin K monomer comprising a variant of the amino acid sequence of SEQ ID NO: 1; wherein the monomer is capable of forming a pore; and
wherein the variant comprises one or more modifications at one or more positions in the region of SEQ ID NO: 1 between about S100 and about K170 which alter the ability of the monomer to interact with an analyte.

36. A monomer according to claim 35, wherein said monomer is as defined in any one of claims 2 to 24.

37. A construct comprising two or more covalently attached monomers derived from Cytotoxin K, wherein at least one of the monomers is a mutant Cytotoxin K monomer as defined in any one of claims 1 to 24.

38. A construct according to claim 37, wherein the monomers are genetically fused or are attached via a linker.

39. A polynucleotide which encodes a mutant Cytotoxin K monomer according to claim 35 or 36 or a construct according to claim 37 or 38.

40. A homo-oligomeric pore comprising a plurality of mutant monomers according to claim 35 or 36; wherein said pore is preferably a heptameric pore.

41. A hetero-oligomeric pore comprising at least one mutant monomer according to claim 35 or 36; wherein said pore is preferably a heptameric pore.

42. A pore comprising at least one construct according to claim 37 or 38.

43. A construct according to claim 37 or 38, or a pore according to claim 41 or 42, wherein at least one monomer in said construct or pore is a monomer of SEQ ID NO: 1.

44. A membrane comprising a pore according to any one of claims 40 to 42.

45. An array comprising a plurality of membranes according to claim 44.

46. A device comprising the array of claim 45, means for applying a potential across the membranes and means for detecting electrical or optical signals across the membranes.

47. Use of a pore according to any one of claims 40 to 42 to characterise a target analyte.

48. Use of a Cytotoxin K pore to characterise a target polypeptide.

49. Use of a Cytotoxin K pore according to claim 48, wherein:
(i) the Cytotoxin K pore comprises a mutant Cytotoxin K monomer according to claim 35 or 36; or
(ii) the Cytotoxin K pore is a pore according to any one of claims 40 to 42.

50. A kit for characterising a target analyte comprising (a) a pore according to any one of claims 40 to 42 and (b) a polynucleotide binding protein or polypeptide handling enzyme.
